(19) United States
(12) Patent Application Publication
(10) Pub. No.: US 2025/0084405 A1
(43) Pub. Date: Mar. 13, 2025

(54) METHOD FOR ANALYZING THE ABILITY OF TARGET NUCLEIC ACID SEQUENCES TO IMPACT GENE EXPRESSION

(71) Applicant: Fred Hutchinson Cancer Center, Seattle, WA (US)

(72) Inventors: Yiting Lim, Seattle, WA (US); Andrew C. Hsieh, Seattle, WA (US)

(73) Assignee: Fred Hutchinson Cancer Center, Seattle, WA (US)

(21) Appl. No.: 18/576,216

(22) PCT Filed: Jul. 7, 2022
(86) PCT No.: PCT/US2022/073511

§ 371 Date: Jan. 3, 2024

Related U.S. Application Data
(60) Provisional application No. 63/219,688, filed on Jul. 8, 2021.

(51) Int. Cl. C12N 15/10

(52) U.S. Cl. CPC C12N 15/1082; C12N 15/1065; C12N 15/1086; C12N 15/1089

(57) ABSTRACT

A method for analyzing an ability of target nucleic acid sequences to impact gene expression is described. In an embodiment, the method includes cloning the target nucleic acid sequences and associated barcode nucleic acid sequences into a plurality of plasmids, sequencing the plasmids to provide long-read sequencing information based on a target nucleic acid sequence of the target nucleic acid sequences and an associated barcode nucleic acid sequence, associating the target nucleic acid sequence with the associated barcode nucleic acid sequence based on the long-read sequencing information, transducing the plurality of plasmids into a plurality of cells, extracting DNA, total mRNA, and polysome-bound mRNA from the cells, sequencing the barcode nucleic acid sequences in the extracted DNA, total mRNA, and polysome-bound mRNA to provide short-read sequencing information, and analyzing the target nucleic acid sequences by comparing the long-read sequencing information and the short-read sequencing information.

### CROSS-REFERENCES TO RELATED APPLICATION

This application claims priority to U.S. Provisional Application No. 63/219,688 filed Jul. 8, 2021, the entire disclosures of which is hereby incorporated by reference.

### STATEMENT OF GOVERNMENT LICENSE RIGHTS

This invention was made with government support under grant number CA230617 awarded by the National Institutes of Health. The government has certain rights in the invention.

### STATEMENT REGARDING ELECTRONIC SEQUENCE LISTING

The Sequence Listing XML associated with this application is provided in XML format and is hereby incorporated by reference into the specification. The name of the XML file containing the sequence listing is 1896-P60WO_Seq_List_20220628] The XML file is 7 pages long; was created on Jun. 20, 2022; and is being submitted via Patent Center with the filing of the specification.

### BACKGROUND

The 5′ untranslated region (5′ UTR) lies within the noncoding genome upstream of coding sequences and plays a pivotal role in regulating gene expression. Encoded within 5′ UTR DNA sequences are numerous cis-regulatory elements that can interact with the transcriptional machinery to regulate mRNA abundance. Furthermore, transcribed 5′ UTRs are composed of a variety of RNA-based regulatory elements including the 5′-cap structure, secondary structures, RNA binding protein motifs, upstream open reading frames (uORFs), internal ribosome entry sites, terminal oligo-pyrimidine tracts, and G-quadruplexes. These elements can alter the efficiency of mRNA translation, and some can also affect mRNA transcript levels via changes in stability or degradation. Individual mutations and single nucleotide polymorphisms in 5′ UTRs have been reported in cancers, including mutations in the 5′ UTRs of oncogenes and tumor suppressors such as c-MYC and p53. Furthermore, individual 5′ UTR mutations in cancer have functional consequences. For example, mutations in the 5′ UTR of the tumor suppressor RB1 alter RNA conformation and mRNA translation in retinoblastoma, while mutations in the 5′ UTR of BRCA1 in breast cancer patients reduce translation efficiency. On a genome-wide scale, recent studies of large patient cohorts have identified recurrent somatic 5′ UTR mutations across a variety of cancers. Moreover, it has been shown that the overall 5′ UTR mutational burden within a cancer may influence malignant phenotypes. Despite evidence pointing to the importance of 5′ UTR mutations in cancer and gene expression dynamics, a systematic functional interrogation of leader sequence mutations at both the transcription and translational levels has yet to be undertaken.

Massively parallel reporter assays (MPRAs) have been employed to dissect the functional consequences of genetic variation in regulatory elements such as promoters and enhancers. These high-throughput technologies have enabled the characterization of these genomic regions on transcriptional activities. This approach has also been used to study UTR elements and their effects on mRNA degradation and translation. These studies have been limited to the investigation of short genomic regions less than 200 bases in length. This is an important limitation because 5′ UTRs range from 18 to more than 3000 bases, and UTR length and sequence context can have dramatic implications on gene expression. Moreover, no studies to date have determined the functional landscape of 5′ UTR mutations across cancer progression at both the transcript and translation levels simultaneously. Thus, current approaches lack the ability to mine the breadth of full-length 5′ UTR activity and the depth of its impact on multiple layers of gene expression. Therefore, there is an urgent need for innovations that can overcome these barriers to allow for the analysis of the functional cancer-associated 5′ UTR-ome.

### SUMMARY

The present disclosure provides a high-throughput approach for multi-layer functional genomics within full-length 5′ UTRs. In various embodiments, the assays of the present disclosure are referred to as PLUMAGE (Pooled full-length UTR Multiplex Assay on Gene Expression). By coupling long-read and short-read sequencing technologies, the methods of the present disclosure overcome the length restriction of traditional MPRAs. Additionally, the methods of the present disclosure can precisely quantify the effects of patient-based somatic mutations on both mRNA transcript levels and mRNA translation efficiency simultaneously, thereby providing an opportunity to interrogate multiple layers of gene expression regulation in cancer. To this end, the Examples of the present disclosure demonstrate functional interrogation of 5′ UTR mutations identified in 229 localized and metastatic prostate cancer patients using PLUMAGE for their impact on mRNA transcript and translation levels. In these Examples, it is observed that 35% of 5′ UTR mutations altered transcript levels or translation rates across the spectrum of prostate cancer. The gene expression changes were driven in part by the creation of promoter elements or by the disruption of RNA-based cis-regulatory motifs. 5′ UTR mutations in MAP kinase signaling pathway genes were identified that are associated with changes in pathway-specific gene expression, responsiveness to taxane-based chemotherapy, and the development of metastases. The functional study of the landscape of 5′ UTR mutations in a human malignancy highlights the molecular implications of this non-coding space in cancer pathogenesis and reveals new nodes of oncogenic gene regulation. In addition, PLUMAGE provides a new technological platform for functional genomics of 5′ UTRs that can be applied to most genetically driven diseases.

Accordingly, in an aspect, the present disclosure provides a method for analyzing target nucleic acid sequences, the method including cloning the target nucleic acid sequences and associated barcode nucleic acid sequences into a plurality of plasmids, sequencing the plurality of plasmids to provide long-read sequencing information based on a target nucleic acid sequence of the target nucleic acid sequences and an associated barcode nucleic acid sequence within a plasmid of the plurality of plasmids. In some embodiments, the method further includes associating the target nucleic acid sequence with the associated barcode nucleic acid sequence based on the long-read sequencing information, transfecting the plurality of plasmids into a plurality of cells, extracting DNA, total mRNA, and polysome-bound mRNA from the plurality of cells, sequencing the barcode nucleic acid sequences in the extracted DNA, total mRNA, and polysome-bound mRNA to provide short-read sequencing information; and analyzing the target nucleic acid sequences by comparing the long-read sequencing information and the short-read sequencing information.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a histogram of genomic distribution of all somatic single nucleotide 5′ UTR mutations in 5 prostate cancer patient derived xenografts (PDX) from the LuCaP series, in accordance with the present technology;

FIG. 1B illustrates a percentage of 5′ UTR mutations in each of LuCaP PDX of FIG. 1A that significantly alter transcript or mRNA translation efficiency (TE) levels, with a false discovery rate (FDR) of less than 0.1, in accordance with the present technology;

FIG. 1C is a volcano plot showing TE fold changes of all 5′ UTR mutations in the LuCaP PDXs of FIG. 1A, in accordance with the present technology;

FIG. 1D shows luciferase assays validating potentially functional 5′ UTR mutations identified by ribosome profiling including ADAM32 (chr8: 38965236, C->T) and COMT (chr22: 19939057, G->A), as well as the negative control ZCCHC7 (chr9: 37120713, C->T), in accordance with the present technology;

FIG. 1E is a simplified schematic of the Pooled full-length UTR Multiplex Assay on Gene Expression (PLUMAGE), in accordance with the present technology;

FIG. 1F illustrates all 30 unique 8-bp barcodes detected and linked with their respective WT and mutant 5′ UTR by PacBio long-read sequencing, in accordance with the present technology;

FIG. 1G is a comparison of mRNA translation efficiency between WT and mutant ADAM32, COMT, and ZCCHC7 5′ UTRs by PLUMAGE, in accordance with the present technology;

FIG. 2A is a comparison of 5′ UTR mutation rate (5′ UTR mutation/Mb) in localized prostate cancer (PCa) patients (n=149) and metastatic castration resistant prostate cancer (mCRPC) patients (n=80), in accordance with the present technology;

FIG. 2B shows KEGG and Reactome pathway analyses of all genes with 5′ UTR and protein coding sequence (CDS) mutations across 229 prostate cancer patients; in accordance with the present technology;

FIG. 2C shows the absolute genomic distance of somatic single nucleotide 5′ UTR mutations within recurrently mutated genes, in accordance with the present technology;

FIG. 2D shows the predicted enrichment of observed 5′ UTR mutations in the patient cohort across known DNA and RNA binding regulatory elements, in accordance with the present technology;

FIG. 2E: shows the predicted enrichment of observed 5′ UTR mutations in the patient cohort across cis-regulatory elements known to affect translation, in accordance with the present technology;

FIG. 3A shows per-gene percentages of distinct barcodes associated with an exact match to an expected 5′ UTR sequence by PacBio long-read sequencing, in accordance with the present technology;

FIG. 3B: is the correlation of normalized read counts per WT and mutated 5′ UTR in each technical and biological replicate for each PLUMAGE DNA sample, in accordance with the present technology;

FIG. 3C is the correlation of normalized read counts per WT and mutated 5′ UTR in each technical and biological replicate for each PLUMAGE total mRNA sample, in accordance with the present technology;

FIG. 3D is the correlation of normalized read counts per WT and mutated 5′ UTR in each technical and biological replicate for each PLUMAGE polysome-bound mRNA sample, in accordance with the present technology;

FIG. 3E shows the proportion of all 5′ UTR mutations assayed by PLUMAGE that showed a significant (FDR<0.1) change in mRNA transcript or translation levels, in accordance with the present technology;

FIG. 3F shows 5′ UTR mutations that significantly change gene expression affect important cancer-related pathways by KEGG pathway analysis, in accordance with the present technology;

FIG. 4A shows 5′ UTR mutations that significantly affect mRNA transcript levels and magnitude fold change compared to unmutated 5′ UTR, in accordance with the present technology;

FIG. 4B shows qPCR validation of the FOS and FGF7 5′ UTR mutations identified by PLUMAGE, in accordance with the present technology;

FIG. 4C is a RNAseq volcano plot of all significantly up and down regulated mRNAs in the human prostate cancer PDX LuCaP 81, in accordance with the present technology;

FIG. 4D shows the FGF7 5′ UTR mutation introducing a thymidine at position chr15: 49715462, which transforms the CACGCG sequence into an E-box motif, in accordance with the present technology;

FIG. 4E is a representative EMSA using the WT versus mutant FGF7 5′ UTR, in accordance with the present technology;

FIG. 5A shows 5′ UTR mutations that significantly affect mRNA translation efficiency and magnitude fold change compared to unmutated 5′ UTRs, in accordance with the present technology;

FIG. 5B shows validation of 5′ UTR mutations in AKT3 and NUMA1 by luciferase assay, in accordance with the present technology;

FIG. 5C shows the C to A 5′ UTR mutation in NUMA1 at position chr11, in accordance with the present technology;

FIG. 5D shows the 5′ UTR mutation in QARS making significant changes in both transcript levels and translation efficiency, not attributable to the amount of DNA transfected, in accordance with the present technology;

FIG. 6A is a schematic showing wildtype (WT, top) and mutant (bottom) versions of CKS2 transcript, including 5′ UTR, normal coding sequence (CDS), and mutant N-terminally extended CDS, in accordance with the present technology;

FIG. 6B is an example method of CRISPR (clustered regularly interspaced short palindromic repeats)-Cas9base editing using evoAPOBEC1-BE4max-NG, in accordance with the present technology;

FIG. 6C shows Sanger sequencing traces from polyclonal population of CRISPR-transfected 293T cells and 6 individual single-cell clones selected from this pool for further study, in accordance with the present technology;

FIG. 6D is a western blot of the 3 WT and 3 CKS2 mutant clonal cell lines created by CRISPR base editing with antibodies against CKS2 and beta-actin, in accordance with the present technology;

FIG. 6E demonstrates that CKS2 qPCR shows no change in mRNA levels between 3 WT and 3 mutant clonal cell lines created from CRISPR base editing, in accordance with the present technology;

FIG. 7A shows genes with 5′ UTR mutations in localized and advanced prostate cancer cluster into distinct functional categories as determined by KEGG pathway analysis, in accordance with the present technology;

FIG. 7B is a heat map of a MAP kinase pathway activity signature demonstrating that patients with functional 5′ UTR mutations to MAP kinase regulators exhibit increased pathway activation compared to non-functional mutations, in accordance with the present technology;

FIG. 7C shows metastatic castration resistant prostate cancer patients harbor 5′ UTR mutations within genes found in the MAP kinase signaling pathway, in accordance with the present technology;

FIG. 7D demonstrated that mCRPC patients with mutated MAP kinase pathway genes are significantly more prone to bone metastases at diagnosis compared to patients who do not harbor these mutations, in accordance with the present technology;

FIG. 7E shows the difference in bone metastasis at diagnosis between the two patient groups is independent of any differences in 5′ UTR tumor mutational burden, in accordance with the present technology;

FIG. 8 shows lengths of all 326 5′ UTRs with somatic mutations in LuCaP PDX samples in accordance with the present technology;

FIG. 9 shows gland enriched normal prostate tissue used for RNAseq and ribosome profiling, in accordance with the present technology;

FIG. 10A shows RNAseq and ribosome profiling of mCRPC PDX tissues, in accordance with the present technology;

FIGS. 10B-10C are dendrograms of normalized read counts for ribosome-bound and total RNA replicates, in accordance with the present technology;

FIG. 10D shows representative periodicity plots of ribosome-bound mRNA and total mRNA from a PDX issue, in accordance with the present technology;

FIG. 10E shows representative periodicity plots showing ribosome bound fragments enriched in one of the three possible codon frames (top) at each base relative to coding start/end, whereas non-protected total mRNA (bottom) is not, in accordance with the present technology;

FIG. 10F shows representative plots of multiple lengths of sequenced reads for ribosome bound (top) and total RNA samples (bottom), in accordance with the present technology;

FIG. 11A is a scatter plot showing correlation of normalized read counts per 8-bp barcode between biological replicates in small PLUMAGE library, in accordance with the present technology;

FIG. 11B is a comparison of performance of a construct without Kozak and ATG sequences by PLUMAGE, in accordance with the present technology;

FIG. 11C is a luciferase assay of construct without Kozak and ATG sequences normalized to the amount of luciferase transcript confirms result seen by PLUMAGE, in accordance with the present technology;

FIG. 12A is a schematic diagram of obtaining 5′ UTR somatic mutations from patient samples, in accordance with the present technology;

FIG. 12B shows the number of read counts per reference (ref) and altered (alt) nucleotide in the 5′ UTRs of tumor and matched normal samples, in accordance with the present technology;

FIG. 12C shows the variant allele frequency (VAF) of all 5′ UTR mutations from tumor and matched normal samples show higher tumor VAFs compared to normal tissues, suggesting reliable detection of somatic 5′ UTR mutations, in accordance with the present technology;

FIG. 12D is a comparison of CDS mutation rates between localized prostate cancer and mCRPC patients shows higher mutation rates in mCRPC patients, in accordance with the present technology;

FIG. 12E shows an overlap in a number of genes with mutations in 5′ UTRs and CDS regions, in accordance with the present technology;

FIG. 12F shows the number of genes with mutations in 5′ UTRs in the Quigley et. al. Cell 2018 dataset compared to the present dataset, in accordance with the present technology;

FIGS. 13A-13B show the most frequently mutated 5′ UTR regulatory elements in prostate cancer, in accordance with the present technology;

FIGS. 14A-14B show determinations of 5′ UTR TSSs and polysome profiling in PLUMAGE experiments, in accordance with the present technology;

FIGS. 15A-15B show quantification of 30-bp barcodes in PLUMAGE, in accordance with the present technology;

FIGS. 16A-16B show polysome to total RNA measurements of translationally regulated PLUMAGE hits correlating with polysome to 80S measurements, and functional 5′ UTR mutations not associated with regional DNA structural changes, in accordance with the present technology;

FIGS. 17A-17B show FOS and FGF7 5′ UTR mutations increase transcript levels independent of mRNA stability and sequence of the randomer barcode, in accordance with the present technology;

FIGS. 18A-18C show different randomer 30-bp barcode used in PLUMAGE that do not impact translation efficiency differences, in accordance with the present technology;

FIG. 19 shows that patients with MAP kinase pathway gene mutations that significantly alter gene expression by PLUMAGE were more sensitive to Taxotere therapy, in accordance with the present technology;

FIG. 20 illustrates an example method for analyzing an ability of target nucleic acid sequences to impact gene expression, in accordance with the present technology;

FIG. 21 illustrates an example method for analyzing target nucleic acid sequences, in accordance with the present technology; and

FIG. 22 illustrates another example method for analyzing target nucleic acid sequences, in accordance with the present technology.

### DETAILED DESCRIPTION

In an aspect, the present disclosure provides methods for analyzing target nucleic acid sequences. In an embodiment, such methods are suitable for analyzing the ability of target nucleic acid sequences to impact gene expression, as set forth in greater detail elsewhere herein.

In an embodiment, the method includes preparing a plurality of plasmids comprising one of a plurality of target nucleic acid sequences and one or more barcode sequences. In this regard, the method can include physically associating target nucleic acid sequences and corresponding one or more barcode nucleic acid sequences into a plasmid. As described further herein, such association between the target nucleic acid sequences and the one or more barcode nucleic acid sequences can be used to analyze how the target nucleic acid sequences impact gene expression, such as through transcription and translation of the target nucleic acid sequences.

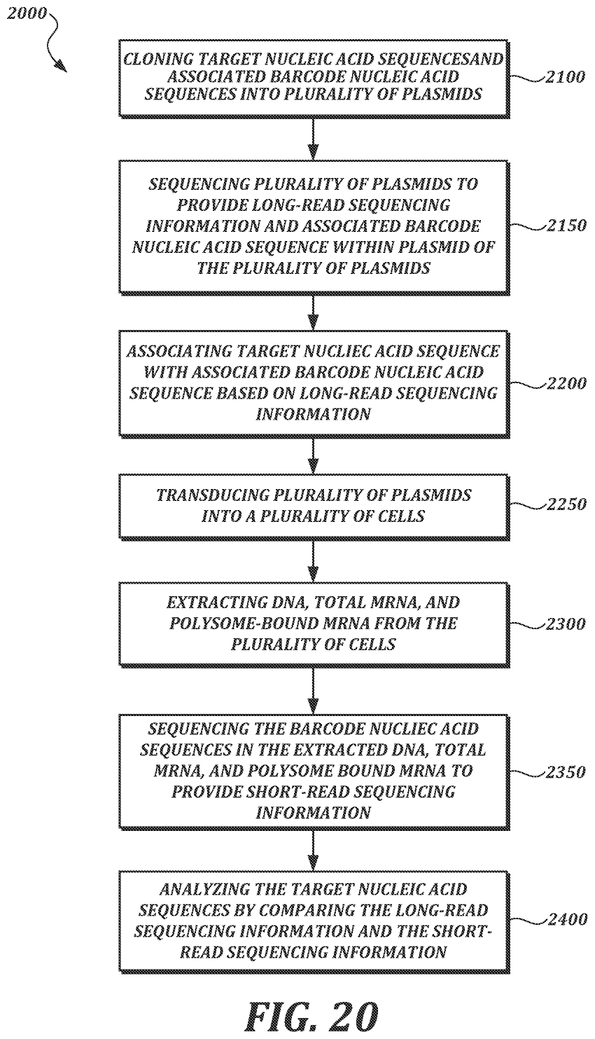

FIG. 20 illustrates an example method 2000 for analyzing an ability of target nucleic acid sequences to impact gene expression, in accordance with the present technology, which will now be described further.

The method 2000 may begin with process block 2100, which includes cloning the target nucleic acid sequences and associated barcode nucleic acid sequences into a plurality of plasmids.

In an embodiment, the method 2000 includes cloning a target nucleic acid and an associated barcode nucleic acid sequence into plasmid, and a second target nucleic acid sequence and a second associated barcode nucleic acid sequence into a second plasmid. These first and second plasmids can be analyzed in parallel as described further herein to assay the first and second target nucleic acid sequences in parallel, such as simultaneously. As above, the plasmids include one or more barcode nucleic acid sequences associated with the target nucleic sequence of an individual plasmid. The one or more barcode nucleic acid sequences are suitable to uniquely identify the target nucleic acid sequence with which it is associated through its physical association with the target nucleic acid sequence. While particular embodiments of the barcode nucleic acid sequences are described, it will be understood that any nucleic acid sequence suitable to uniquely identify the target nucleic acid sequence with which it is associated may be used. In an embodiment, the barcode nucleic acid sequences include nucleic acid sequences selected from the group consisting of a random nucleic acid sequence, a concatenation of a plurality of barcode nucleic acid sequences, and combinations thereof. In some embodiments, the barcode nucleic acid sequences have a length of 8 base pairs. In some embodiments, the barcode nucleic acid sequences have a length of 30 base pairs. In an embodiment, the random nucleic acid sequence has a length in a range of about 5 base pairs and about 50 base pairs, about 5 base pairs to about 30 base pairs, or about 15 base pairs to about 30 base pairs. In an embodiment, the random nucleic acid sequence has a length in a range of about 15 to 50 base pairs.

In an embodiment, where the barcode nucleic acid sequence is a random sequence, the method further includes constraining the library of possible barcode nucleic acid sequences, and wherein these constrained barcode nucleic acid sequences are then transduced into the plurality of plasmids.

In an embodiment, the plasmids of the plurality of plasmids include one or more additional sequences. In an embodiment, the plasmids of the plurality of plasmids include a promoter sequence configured to aid in transcription of the plasmid. In an embodiment, such as where the target nucleic acid sequence is a UTR, such as a 5′ UTR, the promoter nucleic acid sequence is disposed at a 5′ end of the target nucleic acid sequence. In an embodiment, the plasmids of the plurality of plasmids further includes a reporter nucleic acid sequence, such as a reporter nucleic acid sequence suitable to provide a detectable signal, such as an optically detectable signal. In an embodiment, the reporter nucleic acid sequence is disposed at a 3′ end of the target nucleic acid sequence, such as where the target nucleic acid sequence is a 5′ UTR. In an embodiment, the reporter nucleic acid sequence is disposed at a 5′ end of the barcode nucleic acid sequence, again where the target nucleic acid sequence is a 5′ UTR. In an embodiment, the plasmid further comprises an enhanced sequence. While specific examples of the relative positioning of subsequences of the plasmids are described, it will be understood that these relative positions may change, such as depending upon the type of target nucleic acid sequence transduced into the plasmid.

Process block 2100 may be followed by process block 2150, which includes sequencing the plurality of plasmids to provide long-read sequencing information based on a target nucleic acid sequence of the target nucleic acid sequences and an associated barcode nucleic acid sequence within a plasmid of the plurality of plasmids. Such long-read sequencing can include traditional Sanger sequencing suitable to provide sequence information based on or providing the sequence of the target nucleic acid sequence and associated barcode. In an embodiment, the long-read sequencing can include Illumina sequencing.

Process blocks 2100 and 2150 may be followed by process block 2200, which includes associating the target nucleic acid sequence with the associated barcode nucleic acid sequence based through long-read sequencing. Such association can include noting a connection between long-read sequencing information based on the target nucleic acid sequence and long-read sequencing information based on the barcode nucleic acid sequence. In an embodiment, the association can include generating a data structure associating portions of the long-read sequence information based on the target nucleic acid sequence and long-read sequencing information based on the barcode nucleic acid sequence. As described further herein, the association between the target nucleic acid sequence with the associated barcode nucleic acid sequence based on the long-read sequencing information can be used to determine levels of translation and/or transcription of the target nucleic acid sequence based on the short-read sequence information discussed further herein.

In an embodiment, the method 2000 includes distinguishing between correctly synthesized target nucleic acid sequences and incorrectly synthesized target nucleic acid sequences. In preparing the library of target nucleic acid sequences and plasmids containing such sequences, some target nucleic acid sequences/plasmids may not be correctly synthesized. Accordingly, in an embodiment, the method includes removing long-read sequence information from the data analyzed that are based on incorrectly synthesized target nucleic acid sequences. In this regard, the long-read sequence information, and analysis based thereon, is not based upon a false or misleading correlation between a barcode (and short-read sequence information based thereon) and an associated barcode.

The target nucleic acid sequences can include any target nucleic acid sequences of which the sort of analysis described herein is desired. In this regard, the target nucleic acid sequences can include nucleic acid sequences that affect or are thought to affect translation and/or transcription of nucleic acid sequences. In an embodiment, the target nucleic acid sequence includes one or more non-coding genomic regions. In an embodiment, the target nucleic acid sequences include one or more untranslated regions (UTRs). In some embodiments, the one or more untranslated regions are selected from a 5′ UTR, a 3′ UTR, and combinations thereof. While UTRs are described herein, it will be understood that other nucleic acid sequences are suitable for analysis by the methods of the present disclosure, such as, but not limited to, coding sequences.

The target nucleic acid sequences can have a number of different lengths and length ranges. In some embodiments, the target nucleic acid sequences has a length in a range of about 40 base pairs to about 3,000 base pairs. In some embodiments, the length may be smaller, such as 18 base pairs in length. In some embodiments, the target nucleic acid sequences have a length in a range of about 200 to 1,000 bp. In an embodiment, the length of the target nucleic acid sequences is limited by synthesis restrictions and a size of the plasmids into which the target nucleic acid sequences are transduced. In an embodiment, an upper limit of the target nucleic acid sequences is about 20 kb, such as based on a limit of a Gibson assembly reaction.

Process block 2250 includes transducing the plurality of plasmids into a plurality of cells, which may follow process blocks 2100, 2150, and 2200. In an embodiment, such transduction is selected from transfection, nucleofection, viral transduction, and combinations thereof.

Process block 2250 may be followed by process block 2300, which includes extracting DNA, total mRNA, and polysome-bound mRNA from the plurality of cells.

Process block 2350 includes sequencing the barcode nucleic acid sequences in the extracted DNA, total mRNA, and polysome-bound mRNA to provide short-read sequencing information, which may follow process block 2300.

Finally, process block 2350 may be followed by process block 2400, which includes analyzing the target nucleic acid sequences by comparing the long-read sequencing information and the short-read sequencing information. As described further herein, the short-read sequencing information is suitable, in conjunction with the long-read sequencing information, to determine translation and transcription of the target nucleic acid sequence. In an embodiment, determination of translation and/or transcription of the target nucleic acid sequence includes analyzing the target nucleic acid sequences by comparing the long-read sequencing information and the short-read sequencing information.

In an embodiment, comparing the long-read sequencing information and the short-read sequencing information comprises associating barcodes detected in the short-read sequencing information from extracted DNA, total mRNA, and polysome-bound mRNA with the target nucleic acid sequences from the long-read sequencing information. As above, in an embodiment, the long-read sequencing information is suitable to provide a connection or association between the target nucleic acid sequence, whereas, in an embodiment, the short-read sequence information is suitable to correlate the barcode sequence with extracted DNA, total mRNA, and polysome-bound mRNA from the plurality of cells.

Accordingly, in an embodiment, analyzing the target nucleic acid sequences further comprises determining a number of target nucleic sequences, a number of RNA molecules translated from the target nucleic acid sequences, and a number of polysome-bound mRNA molecules from the long-read nucleic acid sequencing information and the short-read sequencing information.

In an embodiment, comparing the long-read sequencing information and the short-read sequencing information comprises associating barcodes detected in the short-read sequencing information from extracted DNA, total mRNA, and polysome-bound mRNA with the target nucleic acid sequences from the long-read sequencing information.

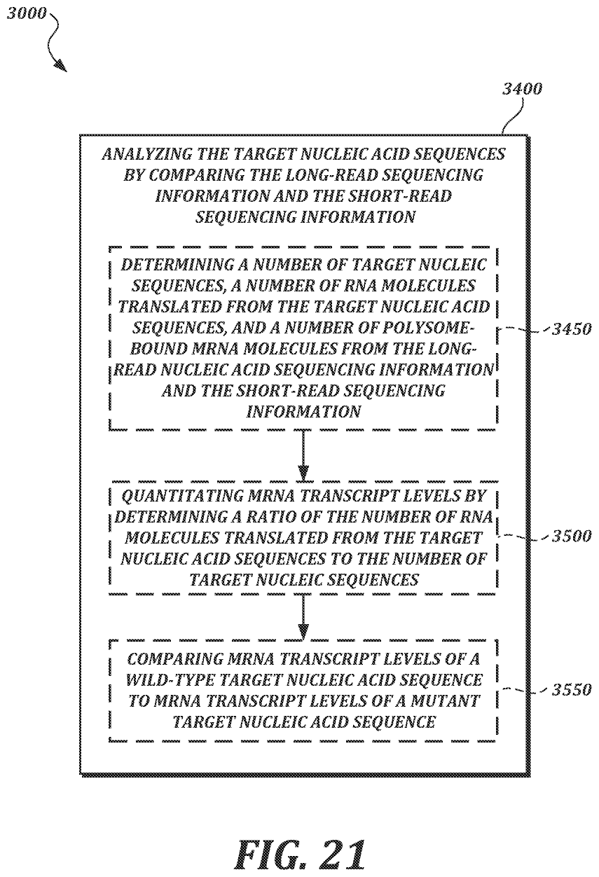

FIG. 21 illustrates an example method 3000 for analyzing target nucleic acid sequences, in accordance with the present technology. In an embodiment, method 3000 is an example of at least a portion of method 2000 described further herein with respect to FIG. 20, such as including process block 2400.

In the illustrated embodiment, method 3000 includes process block 3400, which includes analyzing the target nucleic acid sequences by comparing the long-read sequencing information and the short-read sequencing information.

Inside process block 3400 is process block 3450. In some embodiments, process block 3450 is optional. Process block 3450 includes determining a number of target nucleic sequences, a number of RNA molecules translated from the target nucleic acid sequences, and a number of polysome-bound mRNA molecules from the long-read nucleic acid sequencing information and the short-read sequencing information.

Process block 3450 may be followed by process block 3500. In some embodiments, process block 3500 is optional. Process block 3500 includes quantitating mRNA transcript levels by determining a ratio of the number of RNA molecules translated from the target nucleic acid sequences to the number of target nucleic sequences.

Process blocks 3450 and 3500 may also be followed by process block 3550. In some embodiments, process block 3550 is optional. Process block 3550 includes comparing mRNA transcript levels of a wild-type target nucleic acid sequence to mRNA transcript levels of a mutant target nucleic acid sequence. In this regard, a comparison between transcript levels of mutant and wild-type target nucleic acid sequences can determine, correlate, or otherwise quantify an affect that a mutation in the mutant target nucleic acid sequence has on transcription of the target nucleic acid sequence.

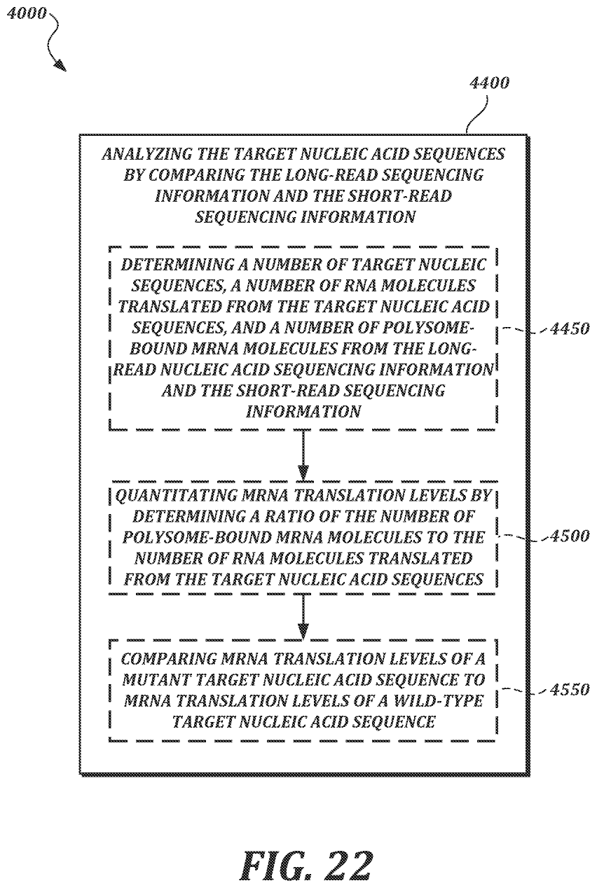

FIG. 22 illustrates another example method 4000 for analyzing target nucleic acid sequences, in accordance with the present technology. In an embodiment, method 4000 is an example of at least a portion of method 2000, such as process block 2400, discussed further herein with respect to FIG. 20.

Process block 4400 includes analyzing the target nucleic acid sequences by comparing the long-read sequencing information and the short-read sequencing information.

Inside process block 4400 is process block 4450. In some embodiments, process block 4450 is optional. Process block 4450 includes determining a number of target nucleic sequences, a number of RNA molecules translated from the target nucleic acid sequences, and a number of polysome-bound mRNA molecules from the long-read nucleic acid sequencing information and the short-read sequencing information.

As a part of process block 4400, process block 4450 may be followed by process block 4500. In some embodiments, process block 4500 is optional. Process block 4500 includes quantitating mRNA translation levels by determining a ratio of the number of polysome-bound mRNA molecules to the number of RNA molecules translated from the target nucleic acid sequences.

Finally, process blocks 4450 and/or 4500 may be followed by process block 4550. In some embodiments, process block 4550 is optional. Process block 4550 includes comparing mRNA translation levels of a mutant target nucleic acid sequence to mRNA translation levels of a wild-type target nucleic acid sequence. In this regard, a comparison between translation levels of mutant and wild-type target nucleic acid sequences can determine, correlate, or otherwise quantify an affect that a mutation in the mutant target nucleic acid sequence has on translation of the target nucleic acid sequence.

The order in which some or all of the process blocks in methods 2000, 3000, and 4000 should not be deemed to be limiting. Rather, one or ordinary skill in the art having the benefit of the present disclosure will understand that some of the process blocks may be executed in a variety of orders not illustrated, or even in parallel.

The Examples described herein use the methods of the present disclosure to describe the functional landscape of somatic 5′ UTR mutations at the transcript and translation levels in prostate cancer. In particular, it is observed that 5′ UTR mutations affect a variety of cancer-associated pathways, some specific to localized while others to metastatic disease. Moreover, these genetic variants are enriched in cis-regulatory elements encoded within specific 5′ UTRs, providing a mechanistic rationale for their existence. Within tumor specimens derived from patients, it is demonstrated that somatic 5′ UTR mutations correlate with changes in transcript levels and translation rates of oncogenic gene targets independent of gene dosage. Moreover, it is observed that 5′ UTR mutations within MAP kinase signaling pathway components are associated pathway activation, response to chemotherapy, and early onset of lethal metastases. These findings implicate somatic alterations to leader sequences as a mechanism for deregulating the flow of genetic information thereby enabling oncogenic levels of gene expression. While 5′ UTR mutations have been identified in a number of cancers, there are still questions as to what the functional relevance of mutations within this non-coding space is and how they alter gene expression. These are key questions because recent studies have shown that the aggregate sum of putative passenger mutations, many of which lie in the 5′ UTR, have clinical consequences. Although these findings point to an important association of these variants with disease, functionally testing all full-length 5′ UTRs and alterations to determine their biological implications is undoubtedly needed. However, this has not been accomplished to date given the inherent limitations of traditional MPRAs as well as the need to quantify changes at both the transcript and translation levels.

In order to fill this experimental and conceptual gap, a functional genomic analysis of patient-based somatic 5′ UTR mutations was conducted across the spectrum of human prostate cancer. This was enabled by the development of PLUMAGE, a new long- and short-read sequencing platform that assays full-length 5′ UTRs in a multiplex manner at both the mRNA transcript and translation levels simultaneously. Using this technology on mutations identified in prostate cancer patients, it was demonstrated that 35% of mutations within the 5′ UTR can increase or decrease transcript levels or translation efficiency. Furthermore, through mechanistic studies, it was found that mutations within leader sequences can re-code their regional nucleotide context to promote oncogenic gene expression. For example, a simple C->T mutation at chr15: 49715462 of the FGF7 5′ UTR was sufficient to increase transcript levels. This increase was mediated through the creation of an E-box motif, which enables MYC:MAX heterodimer binding. Importantly, massively parallel reporter assay results are congruent with endogenous gene expression changes using CRISPR base editing of a C->T mutation in the 5′ UTR of CKS2. This mutation created a uAUG that increased translation of the mRNA in its endogenous context. Of note, while many 5′ UTR mutations studied ablate or create new cis-elements, alterations that were not associated with any known motifs and yet still cause changes in transcript abundance or translation efficiency were found. In this context, these point mutations may instead affect local mRNA structure or epitranscriptomic marks, which can have profound changes on RNA metabolism and ribosome loading.

Described herein is a new resource and technology for multi-layer functional genomic studies of genetic diseases. The versatility of the PLUMAGE methodology allows for customization to study cell-type specific regulation of non-coding elements through lentiviral transduction. The assay can also be adapted to interrogate diverse variants in a variety of genomic regions, such as functionally characterizing all polymorphisms or variants of unknown significance in both the coding and non-coding genomic space. Thus, as a technological resource, PLUMAGE is poised to unlock previously untapped frontiers of human genetics.

Somatic 5′ UTR mutations impact transcript levels and mRNA translation in human prostate cancer. Localized prostate cancer is a highly prevalent disease and can evolve into metastatic castration resistant prostate cancer (mCRPC), which is uniformly lethal. While DNA and RNA-based studies of human tissues ranging from localized to metastatic prostate cancer have been reported, the majority have focused on distant DNA-based regulatory regions or protein coding regions. As such, little is known about the mutational landscape of the 5′ UTR across the spectrum of human prostate cancer. Furthermore, it is unknown if 5′ UTR mutations influence transcript levels or mRNA translation in tumor tissues. To address these questions, all somatic 5′ UTR single nucleotide variants were searched for in a cohort of five primary mCRPC patient-derived xenografts (PDX) belonging to the LuCaP series, which encompass major genomic and phenotypic features of human prostate cancer, including adenocarcinoma (LuCaP 78, LuCaP 81, LuCaP 92), neuroendocrine prostate cancer (LuCaP 145.2), and a hypermutated prostate cancer (LuCaP 147).

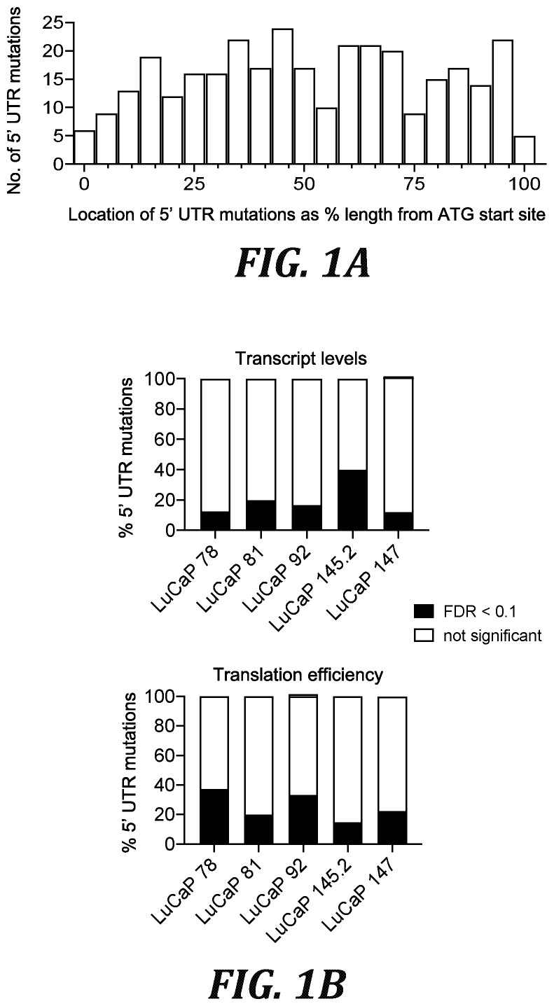

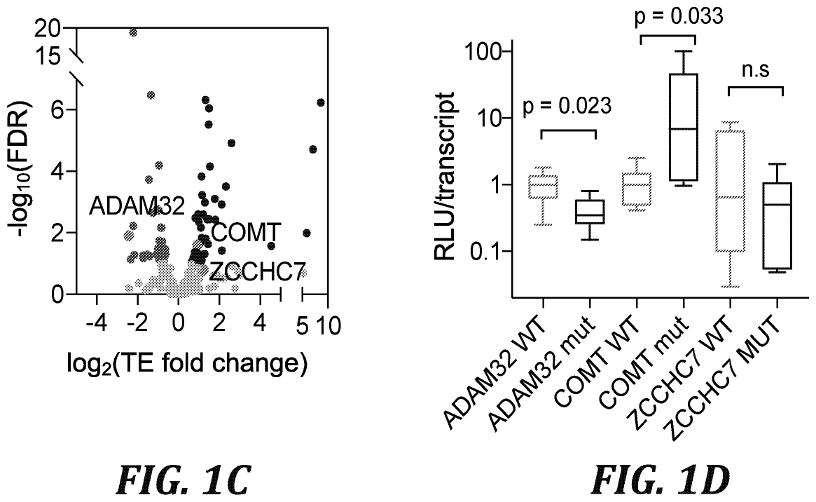

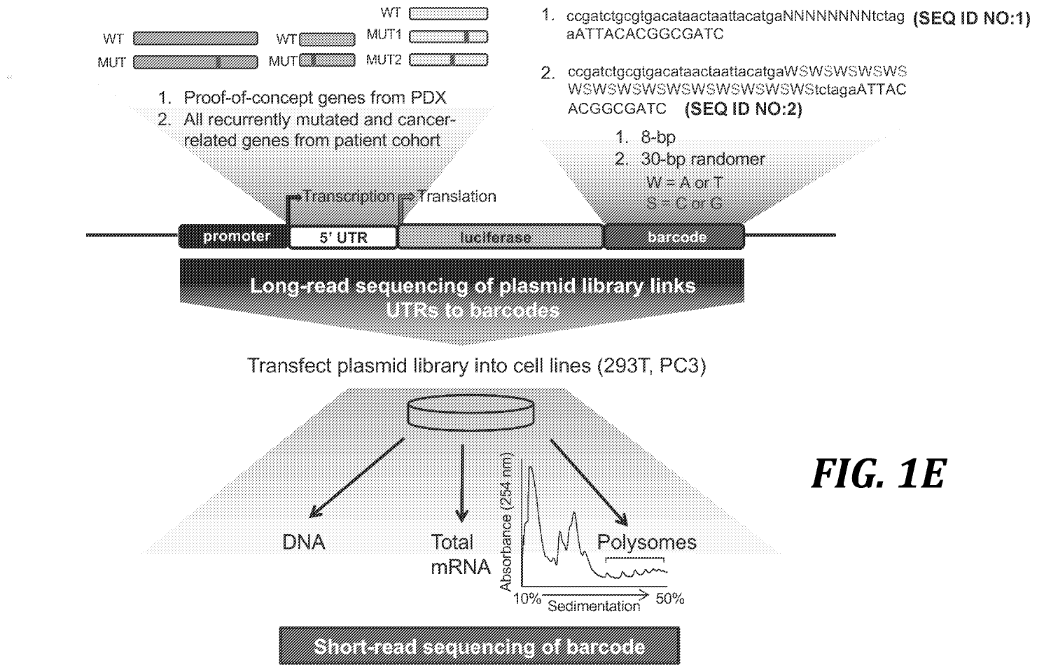

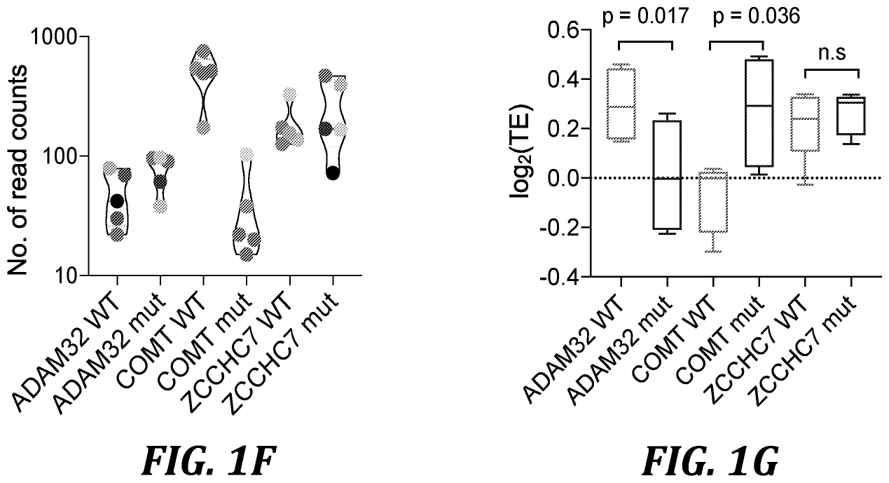

FIG. 1A is a histogram of genomic distribution of all somatic single nucleotide 5′ UTR mutations in 5 prostate cancer patient derived xenografts (PDX) from the LuCaP series, in accordance with the present technology.

FIG. 1B is a percentage of 5′ UTR mutations in each LuCaP PDX of FIG. 1A that significantly alter transcript or mRNA translation efficiency (TE) levels (FDR<0.1), in accordance with the present technology. Across five xenografts representing a spectrum of advanced human prostate cancer (adenocarcinoma, neuroendocrine prostate cancer, hypermutated prostate cancer), 13 mutations exhibited a decrease in transcript levels, while 35 mutations exhibited an increase. At the level of translation, 31 5′ UTR mutations decreased ribosome occupancy (decreased translation efficiency [TE]), while 42 had the opposite effect, independent of changes at the mRNA level.

FIG. 1C is a volcano plot showing TE fold changes of all 5′ UTR mutations in the LuCaP PDXs of FIG. 1A, in accordance with the present technology. Each dot represents TE fold change of a 5′ UTR mutation; dots on the left side of the volcano graph are 5′ UTR mutations that significantly downregulate TE of its specific mRNA (FDR<0.1), dots on the right side of the volcano graphs are 5′ UTR mutations that significantly upregulate TE of its specific mRNA (FDR<0.1). 31 5′ UTR mutations decreased ribosome occupancy (decreased translation efficiency [TE]), while 42 mutations increased TE. Mutations selected for orthogonal validation are labeled with the gene name.

FIG. 1D shows luciferase assays validating potentially functional 5′ UTR mutations identified by ribosome profiling including ADAM32 (chr8: 38965236, C->T) and COMT (chr22: 19939057, G->A), as well as the negative control ZCCHC7 (chr9: 37120713, C->T), in accordance with the present technology. Normalization was performed by taking the ratio of the relative luminescence unit (RLU) to the amount of luciferase transcript determined by qPCR (n=3 biological replicates, Student's t-test).

FIG. 1E is a simplified schematic of the Pooled full-length UTR Multiplex Assay on Gene Expression (PLUMAGE), in accordance with the present technology.

FIG. 1F illustrates all 30 unique 8-bp barcodes detected and linked with their respective WT and mutant 5′ UTR by PacBio long-read sequencing, in accordance with the present technology; (average of 39.4-254.2 read counts per 5′ UTR-barcode pair). Each dot represents a unique 8-bp barcode.

FIG. 1G is a comparison of mRNA translation efficiency between WT and mutant ADAM32, COMT, and ZCCHC7 5′ UTRs by PLUMAGE, in accordance with the present technology. The results are concordant with ribosome profiling and luciferase assay findings in FIGS. 1C and 1D. Normalized polysome read counts for each barcode per construct were taken as a ratio over normalized total RNA read counts for the same barcode (n=5 biological replicates, Student's t-test.).

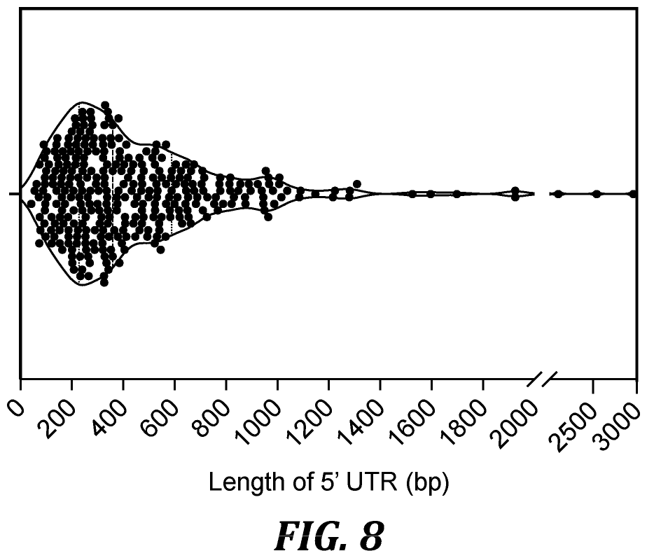

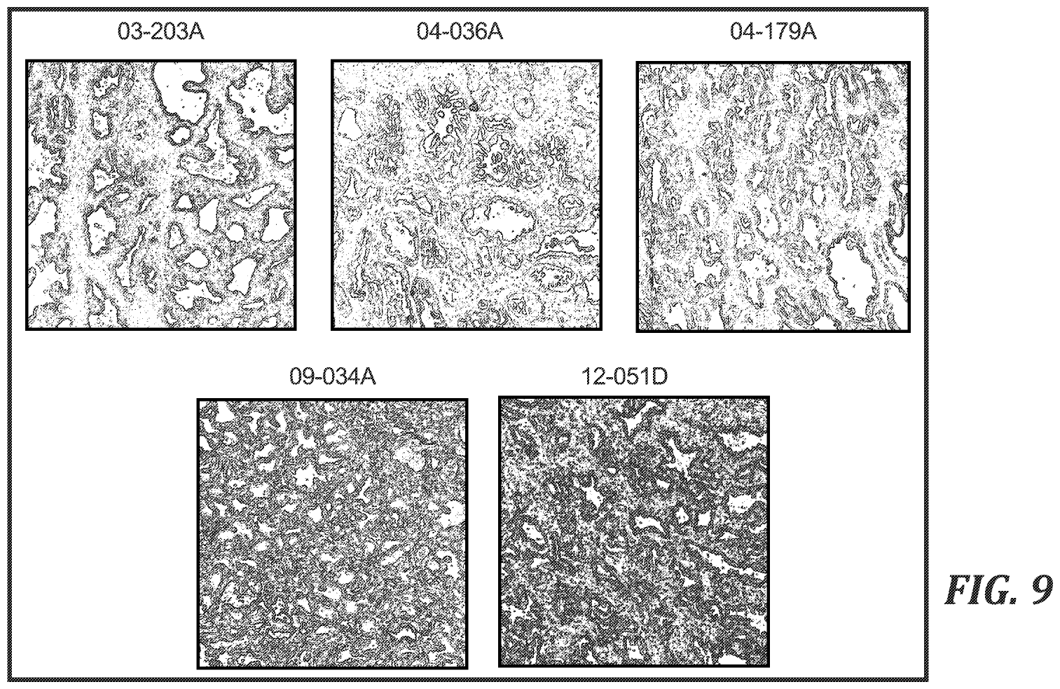

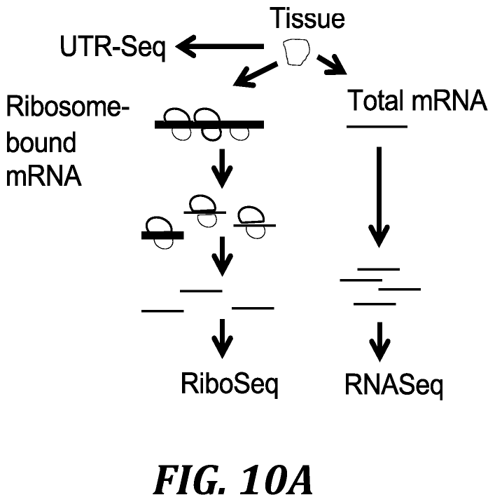

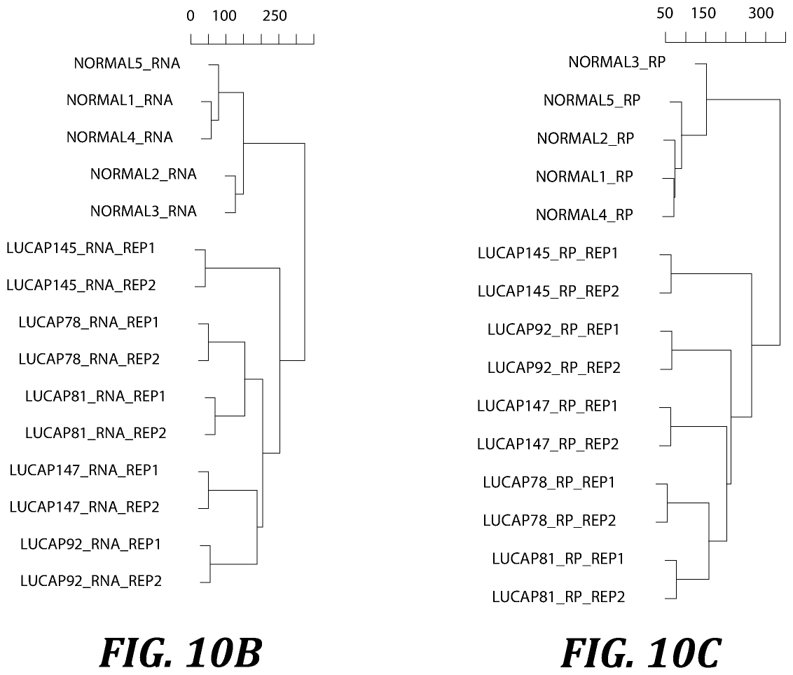

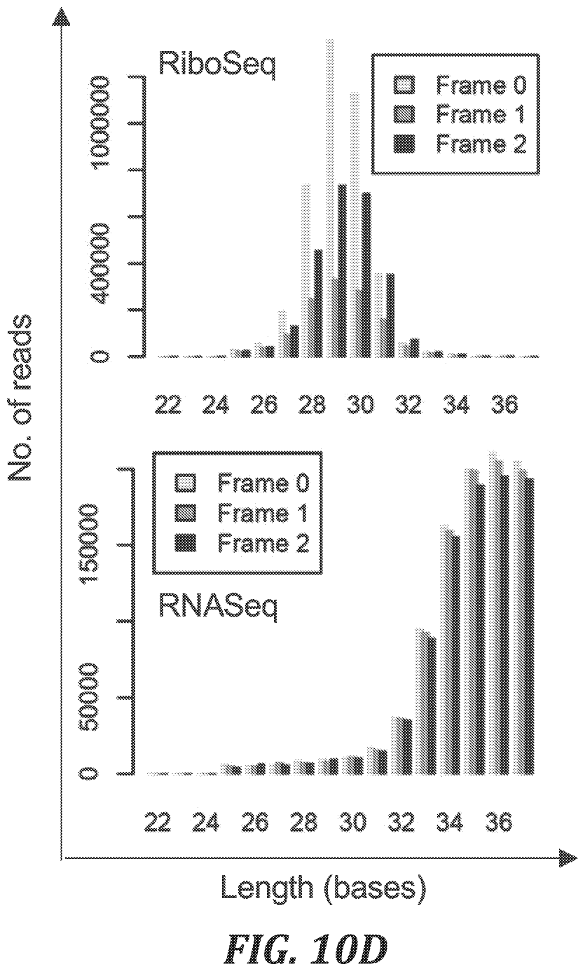

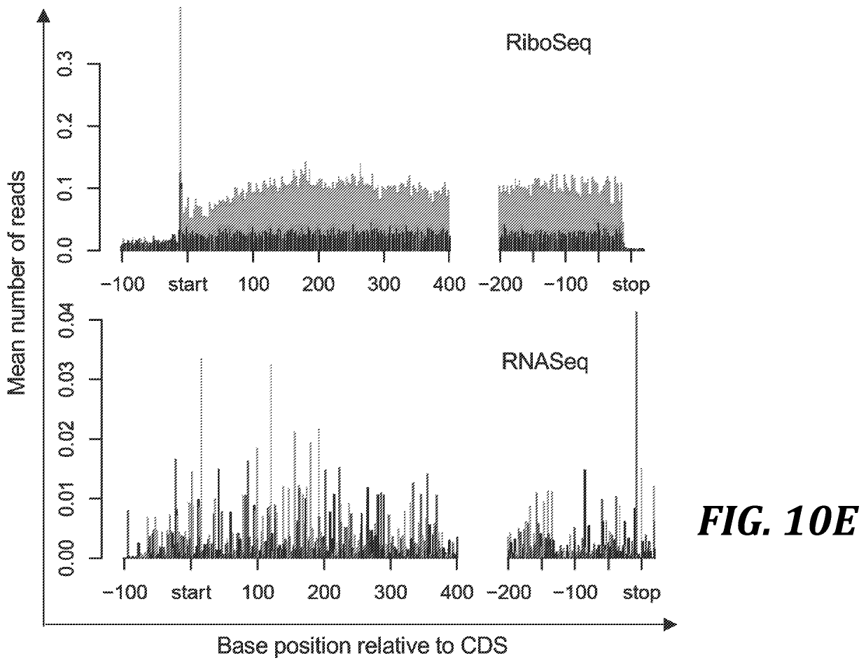

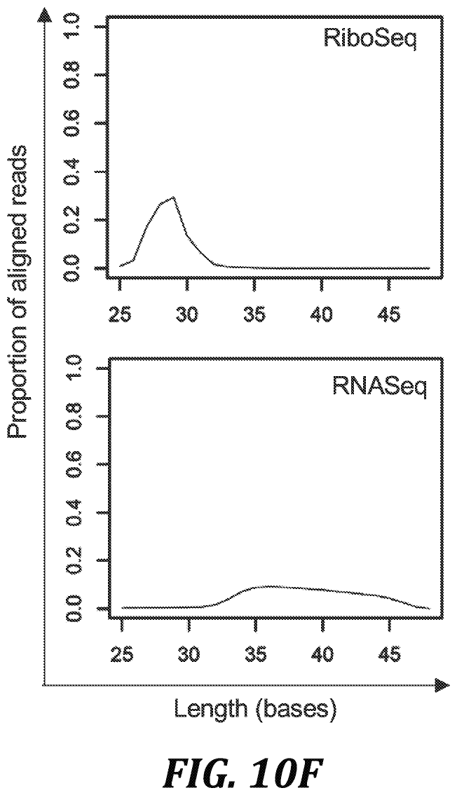

A total of 326 mutations across all five PDXs were observed, with the majority coming from LuCaP 147. These mutations did not localize to a particular region of the 5′ UTR relative to the ATG start codon (FIG. 1A) and were found in 5′ UTRs ranging from 42 to 2960 bases in length (see FIG. 8), suggesting the importance of assaying these mutations in the context of full-length 5′ UTRs. To determine if these 5′ UTR mutations were associated with changes in gene expression, biological replicates of each LuCaP xenograft were compared to a cohort of normal prostate tissues of high glandularity using human tissue-based transcriptome analysis and ribosome profiling (a whole genome method to obtain a global snapshot of all translating mRNAs) (FIGS. 9 and 10A). High reproducibility was observed between replicates, and successful capture of ribosome-bound fragments (FIGS. 10B-10F). The analyses uncovered that out of a total of 326 5′ UTR mutations in five LuCaP xenografts, 12% to 40% of mutations per xenograft were associated with an increase or decrease in transcript levels (FDR<0.1, FIG. 1B). Similarly, at the level of translation, 15% to 38% of 5′ UTR mutations per xenograft were significantly associated with alterations in mRNA translation efficiency (TE) (FDR<0.1, FIG. 1B). In total, 37% of 5′ UTR mutations had a positive or negative impact on transcript levels or translation in a mutually exclusive manner, suggesting multi-layer regulation of gene expression. To reinforce the findings that the gene expression changes were attributed to the single nucleotide mutations and not copy number alterations, copy number analysis of all mutated loci and found that 33% of all copy neutral mutations were analyzed, and also showed significant changes in gene expression. Given that the tissue-based studies only provide an association between 5′ UTR mutations and gene expression changes, and that these changes could be caused by other types of genetic alterations or non-cancer cell types, the ability of specific 5′ UTR mutations to alter protein abundance were functionally tested using an orthogonal luciferase assay. Mutations in ADAM32 (chr8: 38965236, C->T), COMT (chr22: 19939057, G->A), and ZCCHC7 (chr9: 37120713, C->T) were focused on, which decreased, increased, or had no effect on mRNA translation, respectively. Luciferase assays of these 5′ UTR mutations confirmed observations from ribosome profiling (FIGS. 1C and 1D). Given the ability of the LuCaP xenografts to capture the diverse molecular composition of advanced human prostate cancer, these data support the concept that a subset of 5′ UTR mutations may alter gene expression in human tissue. Furthermore, the consequences of these mutations can be observed at both the transcript and translation levels in human-derived tumors.

Given the implications of these findings on the understanding of multi-level gene regulation in cancer, the functional investigation of 5′ UTR mutations genome-wide in a larger cohort of prostate cancer patients was expanded. Although ribosome profiling is a powerful method, it is laborious and low throughput, and would be challenging to implement on hundreds of patient samples. Furthermore, chromosomal alterations such as copy number changes and cell-type heterogeneity would make it difficult to definitively infer causality to single point mutations within the 5′ UTR. Massively parallel reporter assays (MPRA) are high-throughput technologies that enable the analysis of transcriptional or translational activities of myriad regulatory elements while controlling for gene dosage. However, historically they have suffered from two significant limitations. First, current MPRAs used to study 5′ UTR functionality are limited to the examination of short regions (50-125 bases) of the UTR-ome15. This is problematic because human 5′ UTRs can be as long as 3000 bases in length and mutations can occur anywhere along their length. Secondly, current MPRA technologies have yet to assay variants based on human cancers and show how such disease variants can regulate both transcript abundance and translation rates.

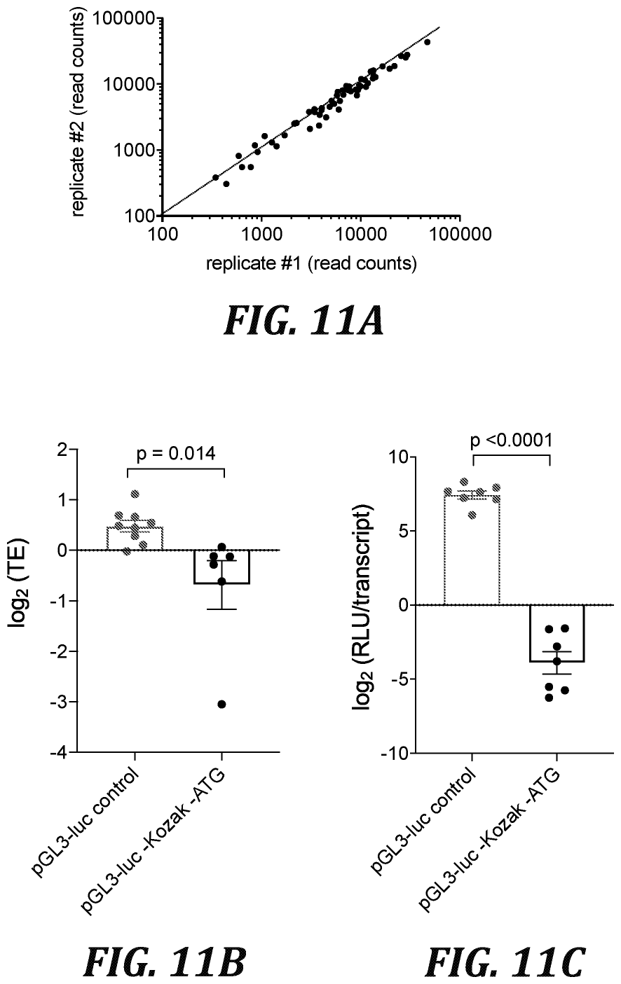

To overcome these limitations, the present disclosure provides, as an example, a method to assess the effects, for example, of prostate cancer patient-based somatic 5′ UTR mutations on mRNA transcript levels and mRNA translation rates in parallel was developed, within the context of each full-length 5′ UTR (FIG. 1E). As a demonstration of its efficacy, a small library of full-length wild-type (WT) and mutant 5′ UTRs were cloned from ADAM32, COMT, and ZCCHC7 (FIGS. 1C and 1D). 5 unique 8-base pair (bp) barcodes were included per UTR variant at the 3′ end of the luciferase protein coding sequence (CDS). To quantify all 5′ UTR-barcode pairs within the library, long-read sequencing was conducted, which identified all plasmids with correct full-length 5′ UTR sequences properly linked to their corresponding unique barcodes (FIG. 1F). This library was transfected into PC3 prostate cancer cells, and 24 hours later extracted DNA, total mRNA, and polysome-bound mRNA (actively translating mRNAs possessing three or more ribosomes) for short-read barcode sequencing. It was observed that biological replicates were highly reproducible across all samples (as shown in FIG. 11A). It was found that the ADAM32 5′ UTR mutation (chr8: 38965236, C->T) decreased while the COMT 5′ UTR mutation (chr22: 19939057, G->A) increased mRNA translation efficiency. The ZCCHC7 5′ UTR (chr9: 37120713, C->T) mutation had no effect (FIG. 1G). These results replicated what was observed through orthogonal tissue-based ribosome profiling and luciferase assays (FIGS. 1C and 1D). To further validate the ability of the approach to quantify changes in gene expression, the Kozak sequence and ATG codon of the luciferase gene was deleted and a decrease in translation efficiency was observed (FIGS. 11B and 11C). This methodology is named the Pooled full-length UTR Multiplex Assay on Gene Expression (PLUMAGE), and it is a new long-read and short-read-linked sequencing approach that enables the massively parallel study of full-length 5′ UTRs and patient-based mutations on gene regulation at the mRNA transcript and translation levels.

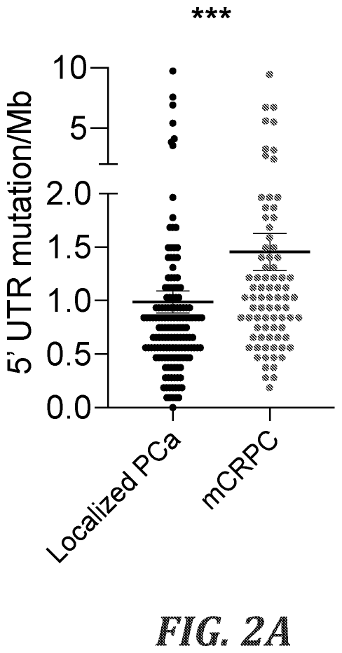

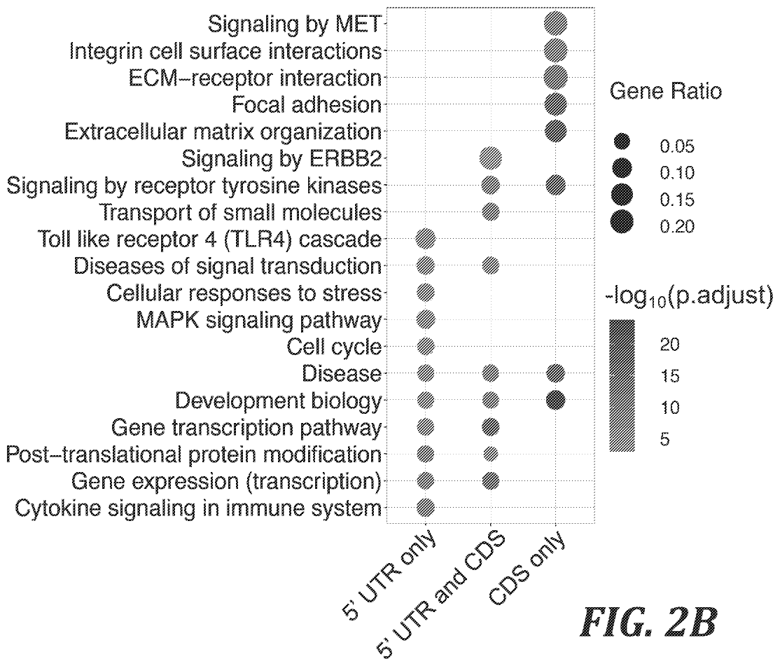

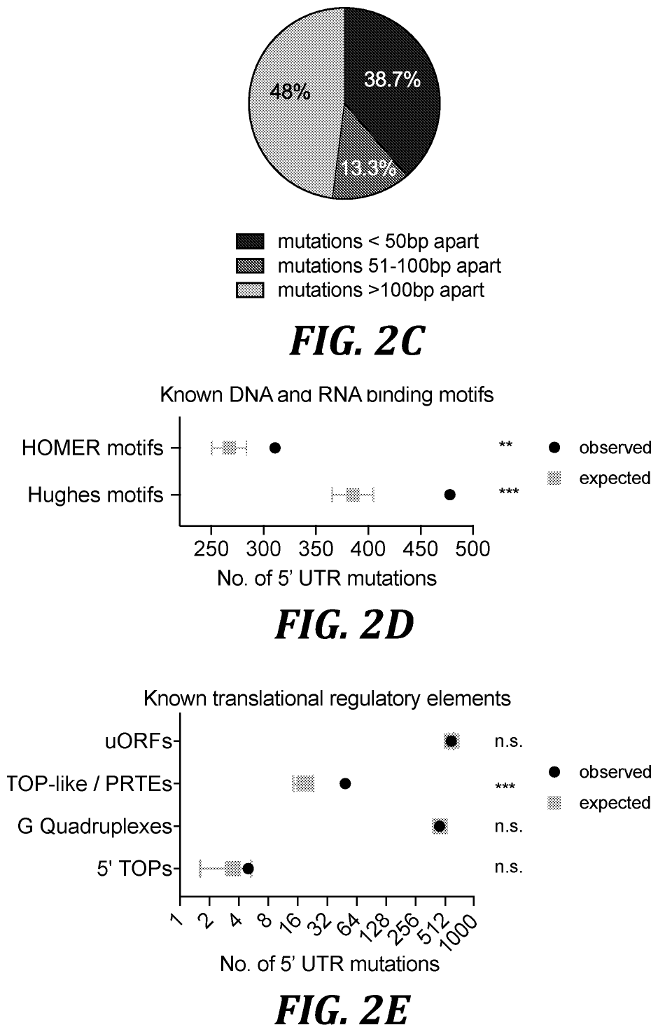

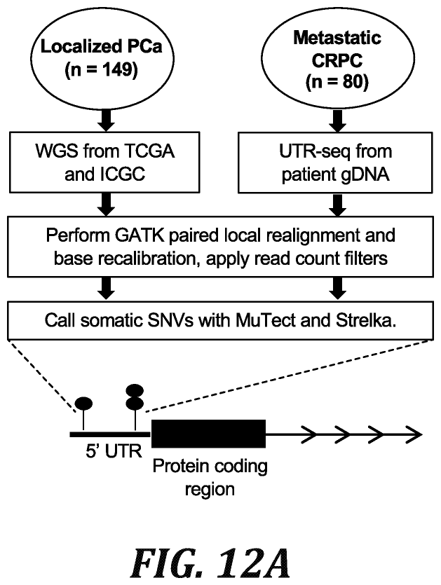

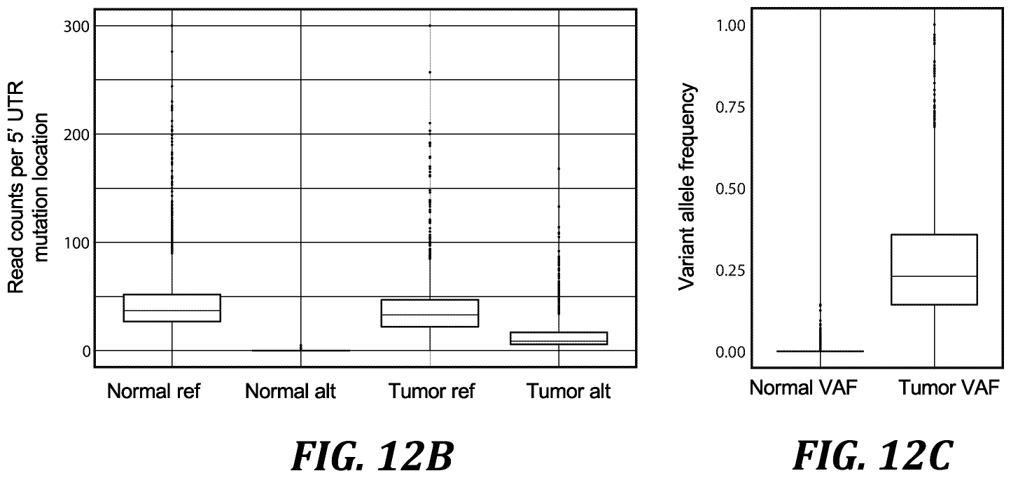

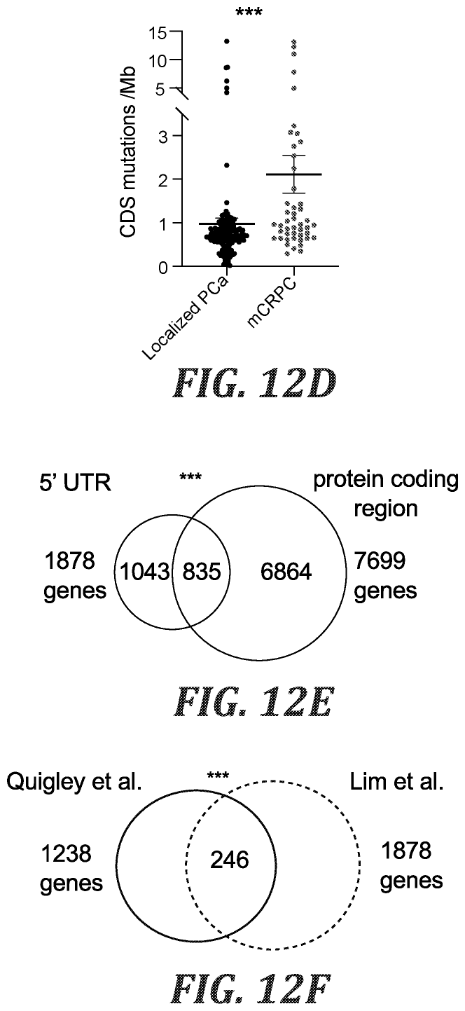

5′ UTR mutations encompassing both localized and metastatic prostate cancer in a large patient cohort were interrogated. Existing whole genome sequencing data from 149 localized prostate cancer patients was analyzed and this cohort was supplemented with newly generated UTR-sequencing of 80 end-stage mCRPC tumors. Collectively, 2200 somatic single nucleotide variants across 1878 genes were identified (as shown in FIGS. 12A-12C). Localized prostate cancers exhibited an average of 0.94 5′ UTR mutations per megabase (mut/Mb), while mCRPCs averaged 1.46 mut/Mb (p<0.001; FIG. 2A), which is consistent with mutation rates observed in the protein coding sequences (CDS) of these patients (p<0.001, FIG. 12D). Of the genes with 5′ UTR mutations, 45% also had alterations in the corresponding coding sequence. However, 55% of 5′ UTR mutations affected genes with no mutations in the protein coding region (p<0.001; FIG. 12E). Interestingly, it was observed that 246 genes with 5′ UTR mutations overlapped with the Quigley et al. mCRPC WGS dataset (FIG. 12F). It was hypothesized that 5′ UTR-specific mutations may affect gene targets that are distinct from those observed in the CDS. To this end, gene set enrichment analysis (GSEA) revealed that 5′ UTR mutations more frequently impacted genes in the MAP kinase signaling and cell cycle pathways, while CDS mutations tend to be enriched in genes involved in cell adhesion processes (FIG. 2B). At a gene specific level, NOTCH227, FTH128, and CDH1229 harbored 5′ UTR mutations and have previously been implicated in prostate cancer pathogenesis. Interestingly, other oncogenic factors not previously implicated in prostate cancer also exhibited 5′ UTR mutations including MECOM and MLF1. Together these findings demonstrate that somatic variants in the 5′ UTR genome appear to affect a diverse group of genes that may be functionally distinct from those that harbor CDS mutations.

Given the diverse array of gene-specific molecular processes that the 5′ UTR regulates, including transcription and mRNA translation, it was reasoned that somatic mutations within the 5′ UTR may be enriched in DNA and mRNA cis-regulatory regions.

FIG. 2A is a comparison of 5′ UTR mutation rate (5′ UTR mutation/Mb) in localized prostate cancer (PCa) patients (n=149) and metastatic castration resistant prostate cancer (mCRPC) patients (n=80), in accordance with the present technology. Each dot represents the mutation rate per patient (*** p<0.001, Mann Whitney test).

FIG. 2B shows KEGG and Reactome pathway analyses of all genes with 5′ UTR and protein coding sequence (CDS) mutations across 229 prostate cancer patients; in accordance with the present technology. Genes with 5′ UTR mutations can cluster with or be independent of genes with CDS mutations (FDR<0.05).

FIG. 2C shows the absolute genomic distance of somatic single nucleotide 5′ UTR mutations within recurrently mutated genes, in accordance with the present technology. 38.7% of recurrently mutated 5′ UTRs have alterations located less than 50-bps apart.

FIG. 2D shows the predicted enrichment of observed 5′ UTR mutations in the patient cohort across known DNA and RNA binding regulatory elements, in accordance with the present technology. Validated DNA (Homer) and RNA protein binding motifs (Hughes) were analyzed. To generate the background (null) distribution of mutations, permutations of all 5′ UTR mutation locations found in our dataset were performed ˜10,000 times taking into account covariates such as trinucleotide context. The total number of observed mutations impacting each regulatory element type was compared to the background distribution of the permutation data and the p-value value was computed (**p<0.01, ***p<0.001).

FIG. 2E shows the predicted enrichment of observed 5′ UTR mutations in the patient cohort across cis-regulatory elements known to affect translation, in accordance with the present technology. The cis-regulatory elements included upstream open reading frames (uORFs), terminal oligo pyrimidine, (TOP)-like or pyrimidine rich translational elements (PRTEs), G quadruplexes, and 5′ TOP elements. To generate the background (null) distribution of mutations, permutations of all 5′ UTR mutation locations found in our dataset were performed ˜10,000 times taking into account covariates such as trinucleotide context. The total number of observed mutations impacting each regulatory element type was compared to the background distribution of the permutation data and the p-value value was computed (***p<0.001).

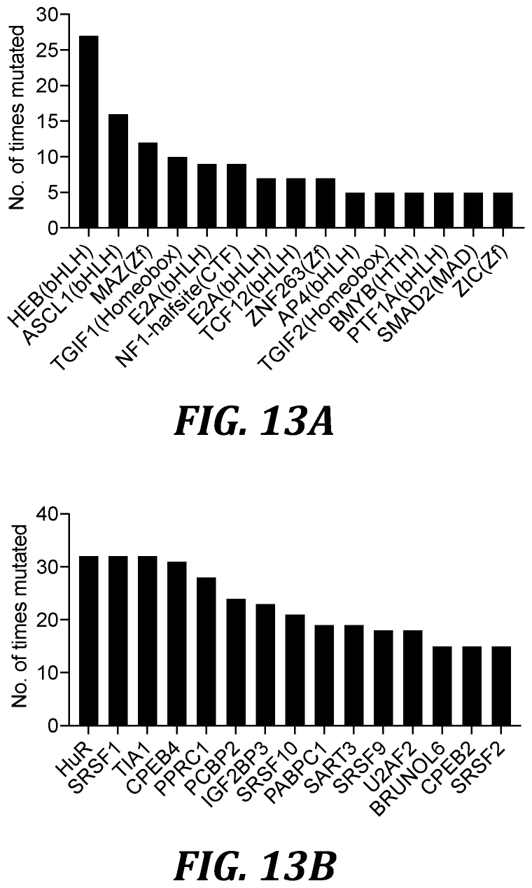

First, the genomic locations of recurrently mutated 5′ UTRs found in 2 or more patients were analyzed, and it was observed that 38.7% of alterations are located within 50-bp of each other (FIG. 2C), suggesting that these clusters of mutations may be targeting functional elements30. Using the HOMER database for DNA binding elements and the Hughes database for human RNA binding protein sites, it was determined that 311 alterations mutated or created new known DNA binding elements, and 478 alterations similarly affected known RNA binding protein sites (FIGS. 2D and 2E). At the DNA level the basic helix-loop-helix (bHLH) motif was the most frequently mutated DNA cis-element, while the HuR, SRSF1, and TIA1 binding motifs were the most frequently mutated RNA-binding sites (as shown in FIG. 13). Next, the goal was to determine if these observed mutations within cis-regulatory elements of 5′ UTRs occur more than would be expected by chance. A background mutation distribution was generated by randomly placing the equivalent number of 5′ UTR mutations found in the analysis into the 5′ UTR-ome, taking into consideration the trinucleotide context of each mutation. This process was simulated 10,000 times. It was observed that mutations that affect DNA and mRNA motifs within the 5′ UTR region of the genome occur more frequently in prostate cancer patients than would be expected by chance (FIG. 2D). Lastly, specific known translation regulatory elements such as upstream open reading frames (uORF), TOP (terminal oligo pyrimidine)-like/PRTE (pyrimidine rich translational element) motifs, G-quadruplexes, and 5′ TOP motifs were queried to determine if somatic mutations are evenly distributed across motifs in general. It was observed that amongst these known 5′ UTR cis-elements, only TOP-like/PRTE motifs exhibited statistically significant enrichment of mutations (p<0.001, FIG. 2E). Overall, these data show that 5′ UTR mutations are present in DNA and RNA cis-elements, suggesting they may functionally impact mRNA transcript levels and mRNA translation.

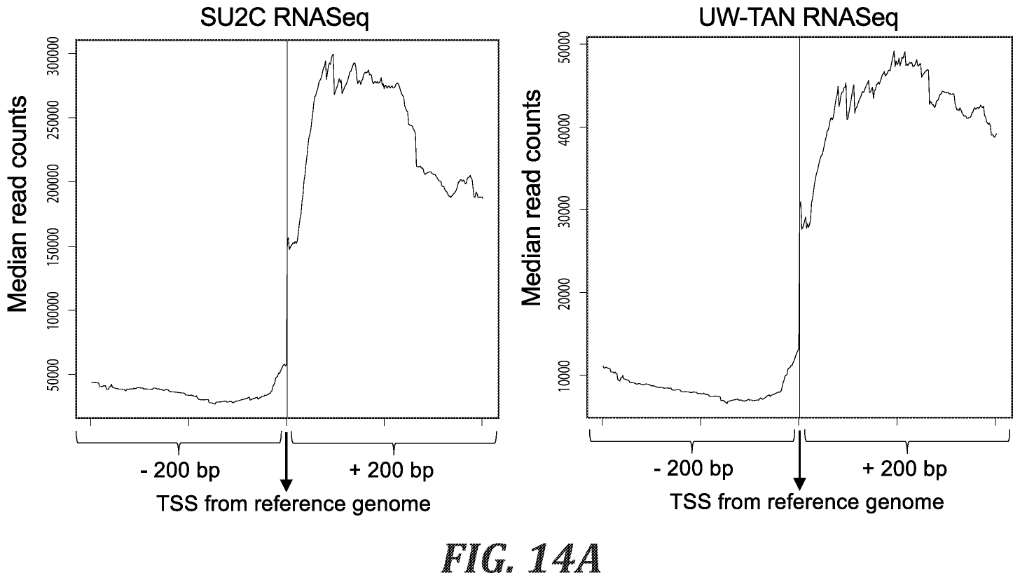

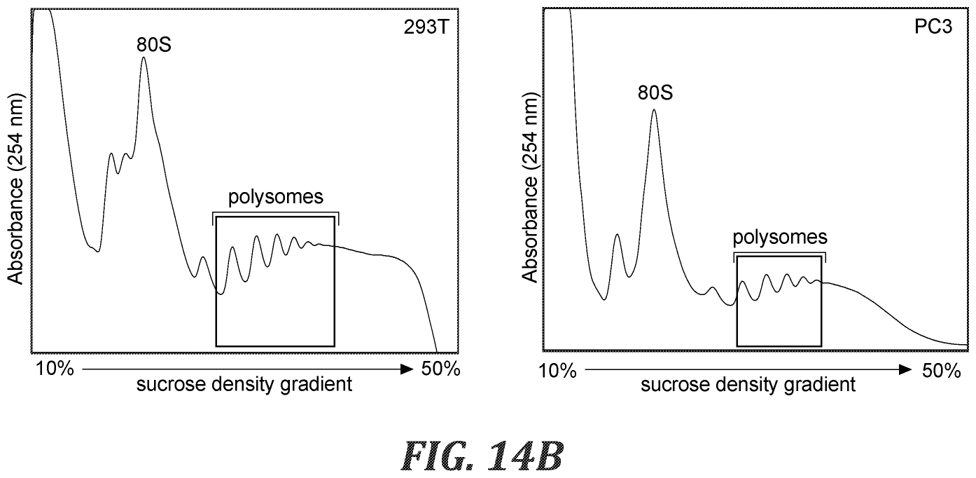

To comprehensively assay the functional landscape of 5′ UTR mutations, a second PLUMAGE library was developed. This larger library was composed of 914 synthesized full-length 5′ UTR sequences covering 545 somatic mutations from all recurrent (2 or more patients) and cancer associated 5′ UTR mutations identified in 229 patients (as shown in FIG. 1E and FIG. 14A).

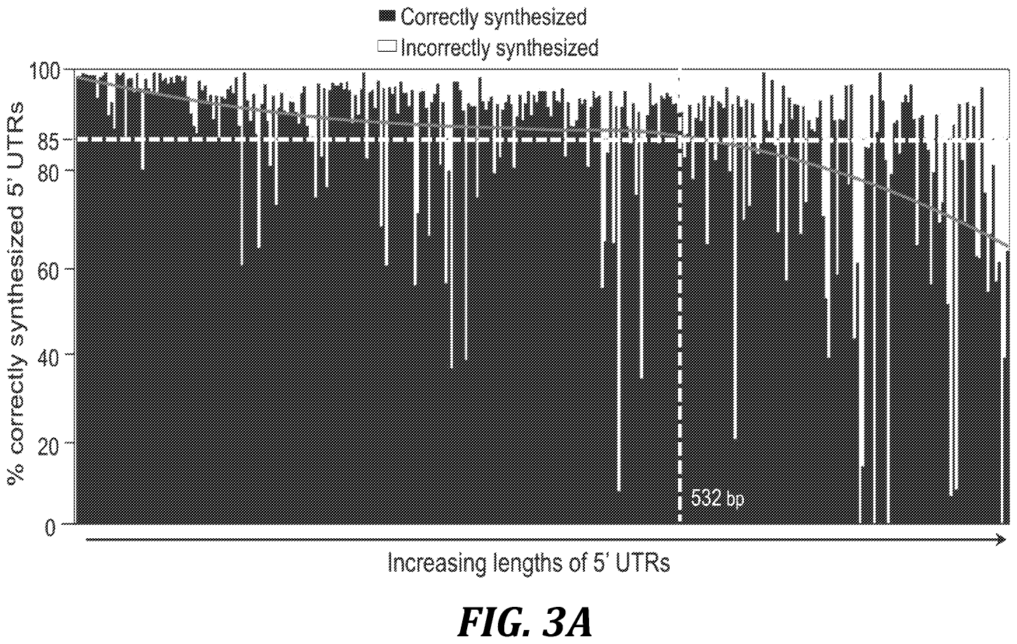

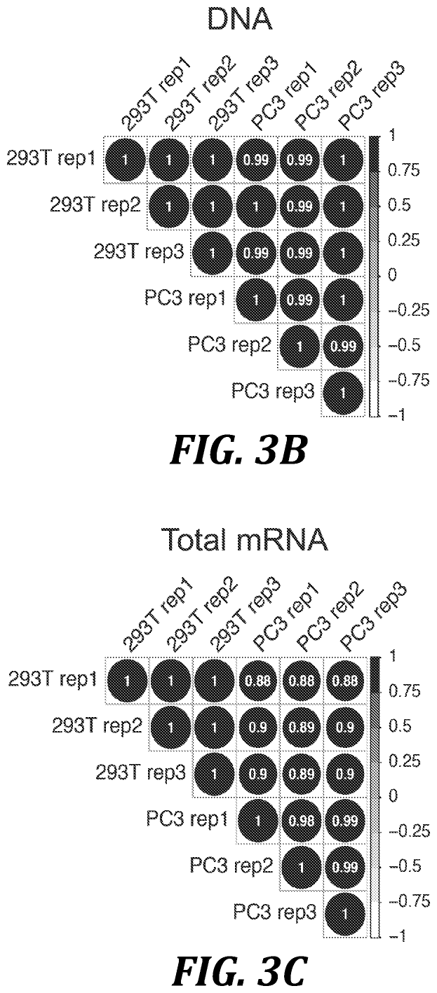

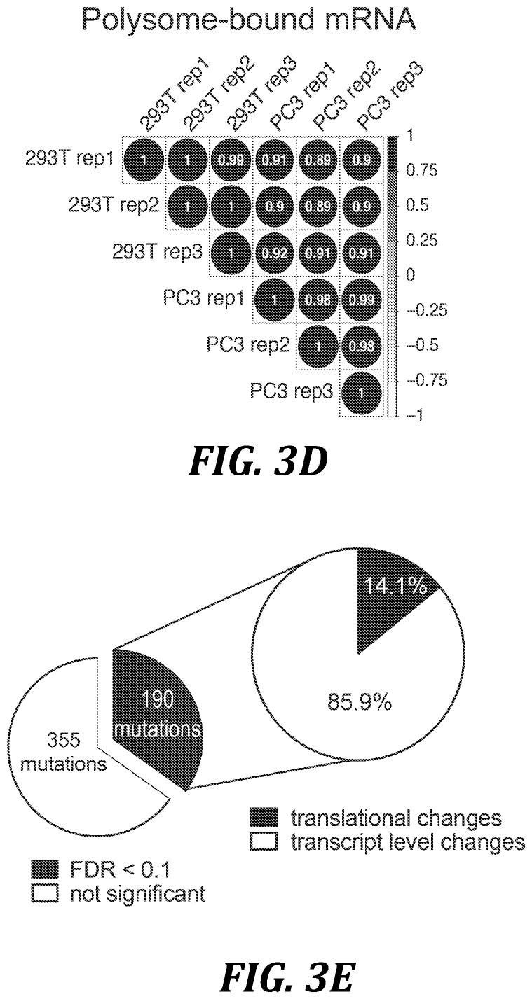

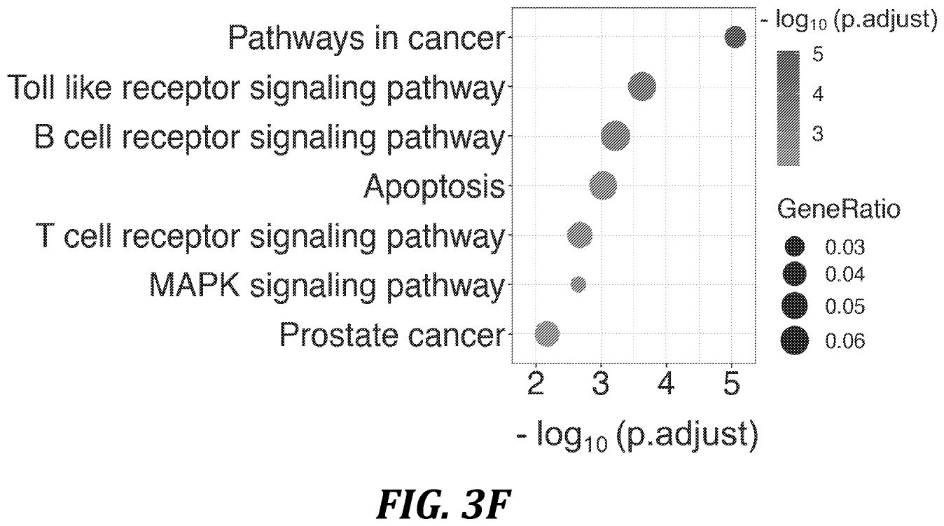

FIG. 3A shows per-gene percentages of distinct barcodes associated with an exact match to an expected 5′ UTR sequence by PacBio long-read sequencing, in accordance with the present technology. All distinct 5′ UTR sequences were observed by long-read sequencing and linked to an average of 236 distinct 30-bp barcodes. For each gene, the percentage of barcodes associated with an exactly matching 5′ UTR are plotted as black vertical bars (correctly synthesized). Genes are ordered by increasing 5′ UTR length from left to right, and the average rate of exactly matching barcodes is marked by a horizontal dashed line at 85%. A smoothed fit, using loess regression, of percentage matching vs. rank order of length is shown as a gray line near the top of the graph.

FIG. 3B is a correlation of normalized read counts per WT and mutated 5′ UTR in each technical and biological replicate for each PLUMAGE DNA sample, in accordance with the present technology. 3 biological replicates were analyzed for each cell line (293T and PC3). The Pearson correlation coefficient was calculated to determine significance and was found to be r>0.99 for all samples (All p-values<0.0001).

FIG. 3C is a correlation of normalized read counts per WT and mutated 5′ UTR in each technical and biological replicate for each PLUMAGE total mRNA sample, in accordance with the present technology; 3 biological replicates were analyzed for each cell line (293T and PC3). The Pearson correlation coefficient was calculated to determine significance and was found to be r>0.8 for all samples (All p-values<0.0001).

FIG. 3D is the correlation of normalized read counts per WT and mutated 5′ UTR in each technical and biological replicate for each PLUMAGE polysome-bound mRNA sample, in accordance with the present technology. 3 biological replicates were analyzed for each cell line (293T and PC3). The Pearson correlation coefficient was calculated to determine significance and was found to be r>0.89 for all samples (All p-values<0.0001).

FIG. 3E shows the proportion of all 5′ UTR mutations assayed by PLUMAGE that showed a significant (FDR<0.1) change in mRNA transcript or translation levels, in accordance with the present technology.

FIG. 3F shows 5′ UTR mutations that significantly change gene expression affect important cancer-related pathways by KEGG pathway analysis (FDR<0.05), in accordance with the present technology. Shown are 190 mutations.

Here, instead of using five 8-base pair barcodes per 5′ UTR, a 30-base pair randomer barcode was cloned downstream of the luciferase CDS. The library was constrained to 212,325 unique barcodes with an average of 236 barcodes per 5′ UTR, which enabled deep sampling of each mutated and unmutated 5′ UTR. To determine the 5′ UTR-barcode pair identities and ensure the analysis of only correctly synthesized 5′ UTRs, long-range sequencing of the entire plasmid library was conducted. Here it was observed that 85% of sequenced plasmids had the correct WT or mutant 5′ UTR sequences. Interestingly, 89.9% of 5′ UTR sequences shorter than 532 bases were correctly synthesized, while only 77.1% of sequences larger than 532 bases were correctly synthesized (FIG. 3A). Thus, the highly saturated barcode system is essential to identify correctly synthesized constructs for further downstream analysis and provides a solution to confidently assay 5′ UTRs of any length. In some embodiments, the method further comprises introducing a plurality of mutations into a plasmid of the plurality of plasmids. In some embodiments, the plurality of mutations are introduced into the plasmid as one or more mutant 5′ UTRs. This allows for PLUMAGE to determine how the plurality of mutation in combination affect gene expression.

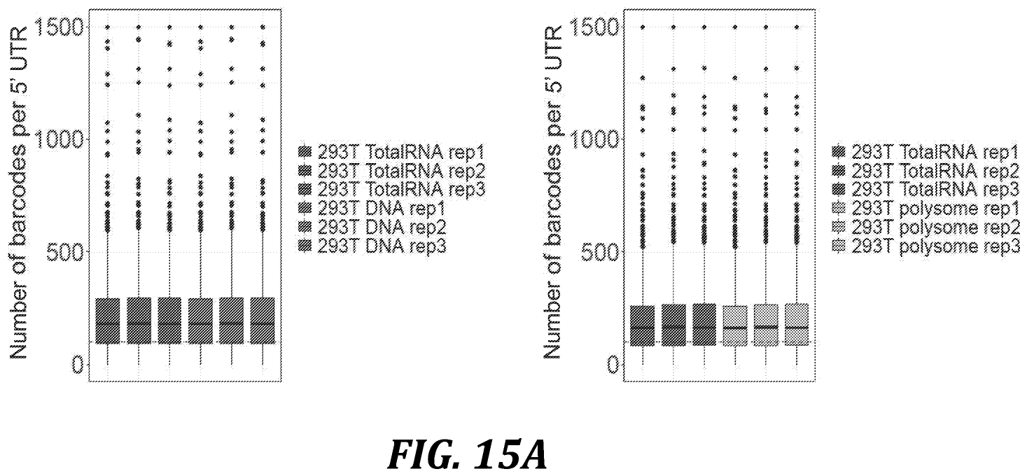

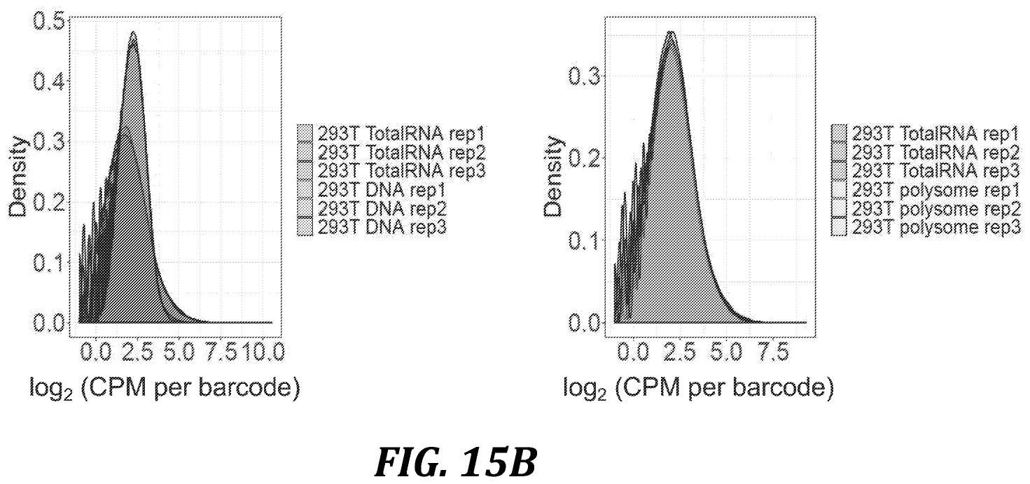

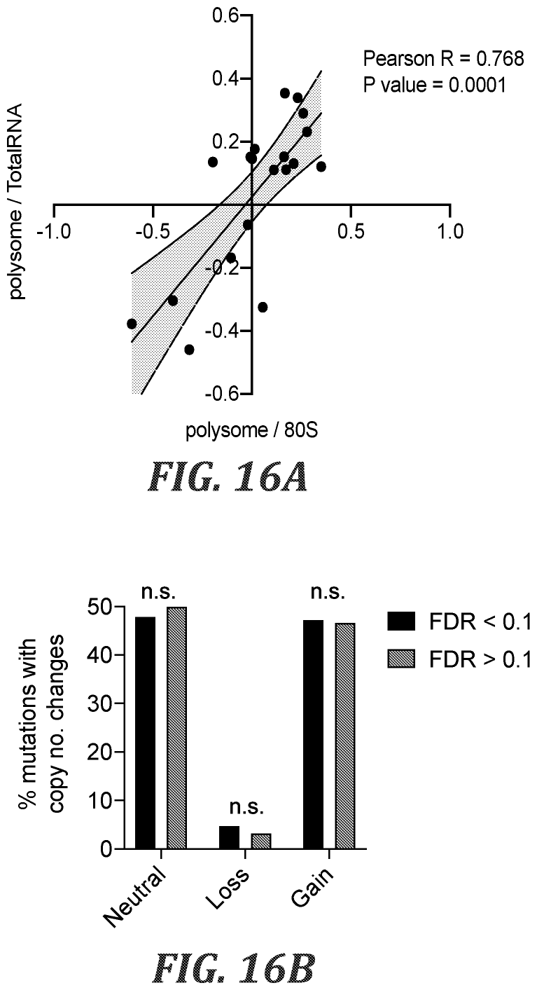

The plasmid library was transfected into human PC3 prostate cancer cells and human embryonic kidney 293T cells. After 24 hours, DNA, total mRNA, and polysome-bound mRNA (mRNA associated with three or more ribosomes) were isolated and sequenced (as shown in FIG. 14B). Short-read sequencing of the 30-bp barcodes in each DNA, total mRNA, and polysome-bound mRNA sample showed a strong correlation across three biological replicates in both cell lines (FIGS. 3B-3D). After filtering for incorrectly synthesized or cloned constructs using the long-read dataset (FIG. 3A), each WT and mutant full-length 5′ UTR was represented by an average of 214 unique 30-bp barcodes (minimum normalized read count of 0.5 counts per million, FIGS. 15A and 15B). Notably, all constructs reliably detected by long-read sequencing were identified in all DNA, total mRNA, and polysome-bound mRNA samples by short-read sequencing. Furthermore, strong correlation was observed between 293T and PC3 cells across all replicates in all samples, suggesting high reproducibility across different cell lines (Pearson r>0.8, p<0.0001; FIGS. 3B-3D). Small differences in correlation were primarily seen between cell lines suggesting that some mutations may have a context-specific dependency. To quantitate changes in mRNA transcript levels and control for gene dosage, the total mRNA barcode read counts were normalized to the corresponding DNA read counts of that particular barcode, and fold change was measured by comparing the mutant 5′ UTR to the WT 5′ UTR. To quantitate changes in mRNA translation, translation efficiency (TE) was measured, which was determined by calculating the ratio of normalized polysome-bound RNA barcodes to their respective total mRNA barcodes, and similarly fold change was measured by comparing the mutant 5′ UTR to the WT 5′ UTR. Out of a total of 545 mutations assayed, 190 showed significant changes in mRNA transcript levels or mRNA translation efficiency (FDR<0.1; FIG. 3E). To ensure that changes at the level of translation were reflective of differential ribosome loading, the polysome to 80S ratio was also measured, which highly correlated with changes in translation efficiency (Pearson r=0.768, p=0.0001; as shown in FIG. 16A). Importantly, these functional mutations affected genes that belong to cancer related pathways and include the oncogenic FTH1 and tumor suppressive MECOM (FIG. 3F). It has been previously reported that germline variants within the 5′ UTR that create cis-regulatory elements, such as uORFs, are under strong negative selection. As such, positive PLUMAGE hits were tested to determine if they were associated with genomic structural variations within the 5′ UTR compared to 5′ UTR mutations that were not functional. Comparing the localized copy numbers of the mutant 5′ UTR on a per-patient bases, no increase in copy number losses were observed as a result of having a functional 5′ UTR mutation (as shown in FIG. 16B). Together, these findings demonstrate that 34.8% of mutated 5′ UTRs harbor functional alterations that can deregulate cancer-related genes in prostate malignancies at either the transcriptional or translational levels.

Orthogonal validation of PLUMAGE reveals functional 5′ UTR mutations that create neo-promoters, disrupt RNA cis-elements, or affect multi-level gene regulation To validate functional 5′ UTR mutations identified through PLUMAGE, individual WT and mutant pairs were tested by orthogonal qPCR and luciferase assays.

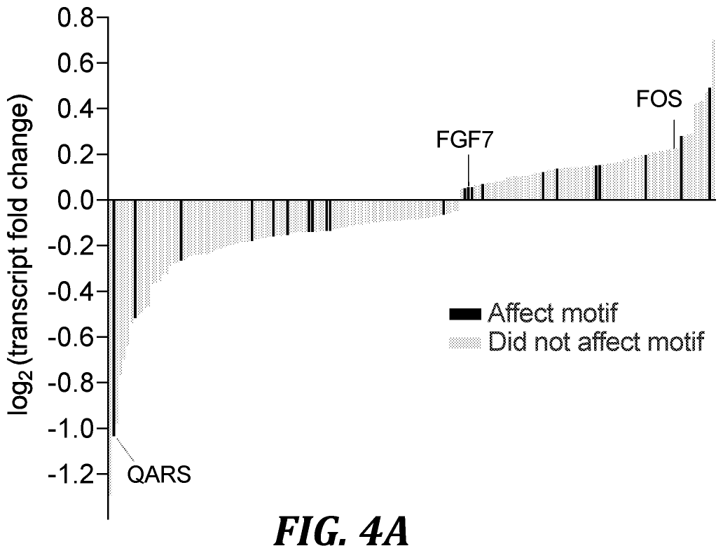

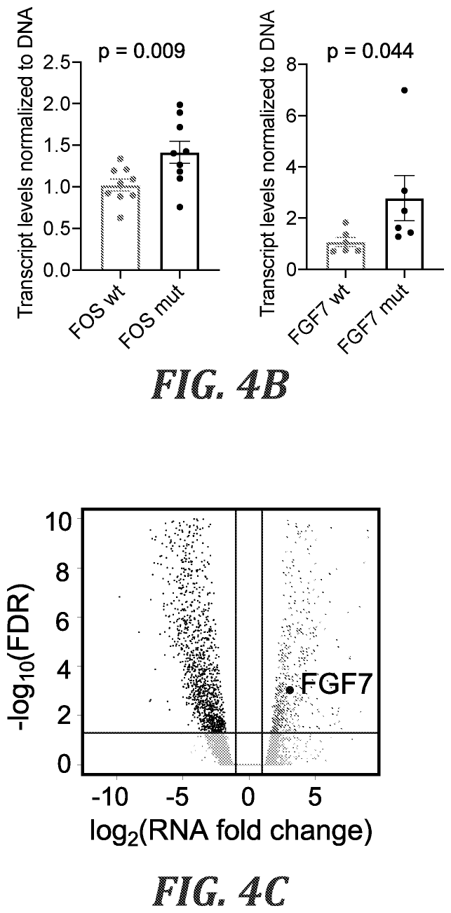

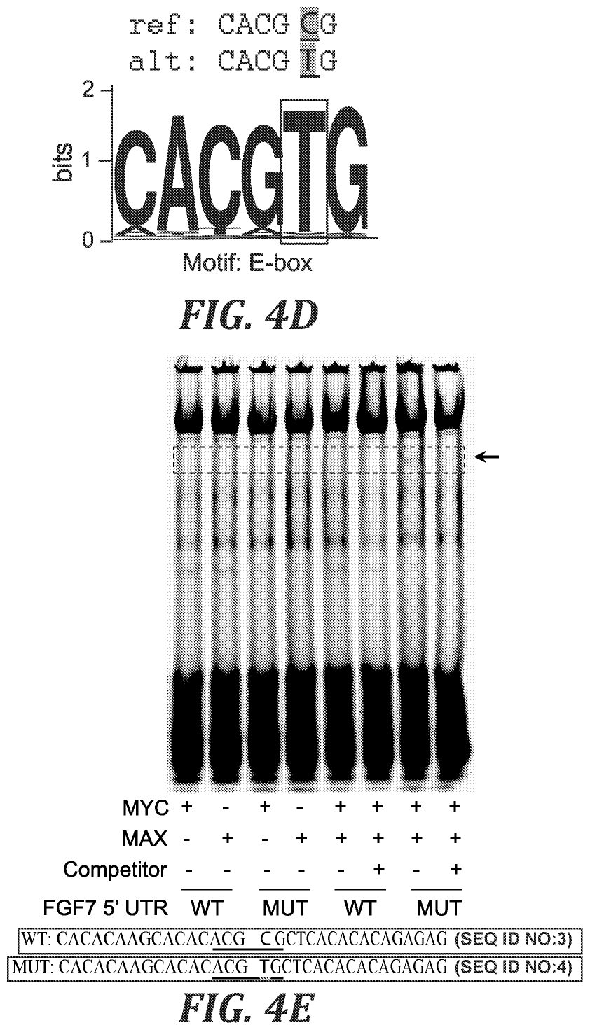

FIG. 4A shows 5′ UTR mutations that significantly affect mRNA transcript levels and magnitude fold change compared to unmutated 5′ UTR, in accordance with the present technology. A proportion of mutations also impact a known DNA binding element, indicated by black bars (Mann-Whitney test, FDR<0.1).

FIG. 4B shows qPCR validation of the FOS and FGF7 5′ UTR mutations identified by PLUMAGE, in accordance with the present technology. WT and mutant 5′ UTRs were cloned into a luciferase reporter construct and transduced into PC3 prostate cancer cells. Luciferase transcript levels were then normalized to luciferase DNA (n=6 to 9 biological replicates, +s.e.m. Student's t-test). The FOS mutation (chr14: 75745674, C->G), and the FGF7 5′ UTR (chr15: 49715462, C->T) were both identified by PLUMAGE.

FIG. 4C is a RNAseq volcano plot of all significantly up and down regulated mRNAs in the human prostate cancer PDX LuCaP 81, in accordance with the present technology (FDR<0.1). Within this PDX, FGF7 exhibits a 5′ UTR mutation at chr15: 49715462, C->T that is associated with an increase in FGF7 transcript levels.

FIG. 4D shows the FGF7 5′ UTR mutation introducing a thymidine at position chr15: 49715462 which transforms the CACGCG sequence into an E-box motif (CACGTG), in accordance with the present technology.

FIG. 4E is a representative EMSA using the WT versus mutant FGF7 5′ UTR, in accordance with the present technology. Labeled probe sequences (33-bp) containing the E-box sequence generated by the mutation in the 5′ UTR of FGF7, and the wild-type sequence are shown. Binding of MYC: MAX heterodimer protein complex is observed only with the mutated oligonucleotide probe containing the E-box sequence. Binding of the labeled oligo can be abolished using an unlabeled competitor;

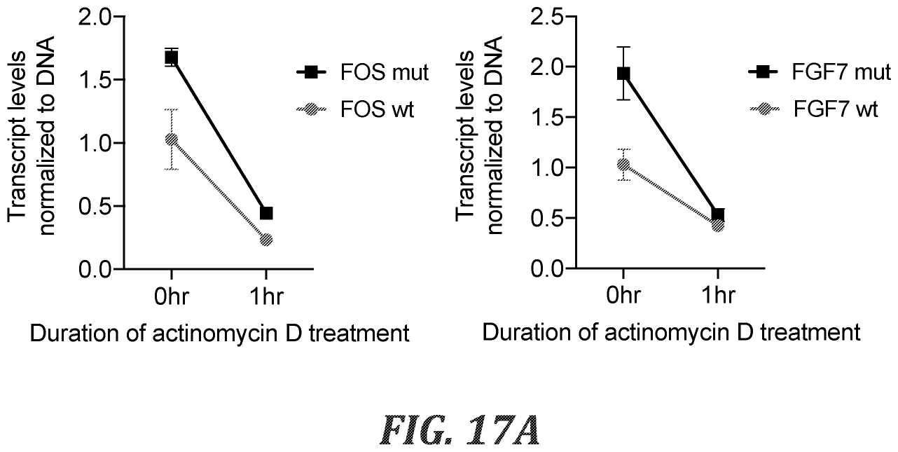

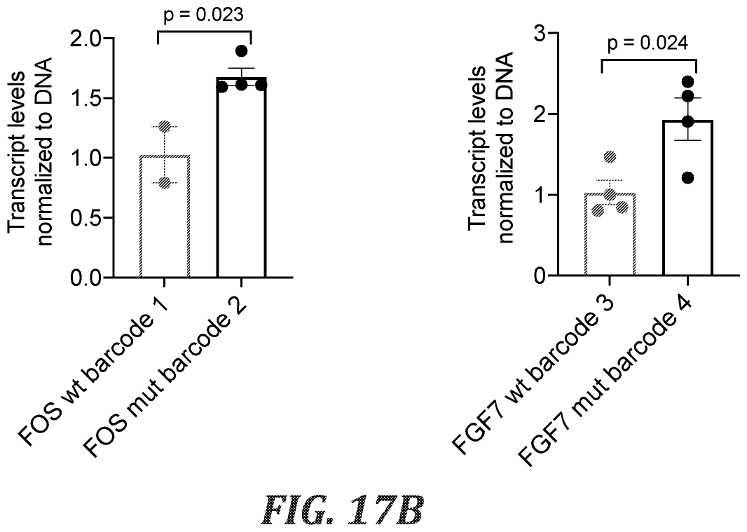

Mutations that impact transcript levels were found in oncogenic genes such as FOS (chr14: 75745674, C->G) and FGF7 (chr15: 49715462, C->T), which are components of the MAP kinase signaling pathway and known to drive prostate cancer pathogenesis (FIG. 4A). A recent study of mCRPC patients reported that MAP kinase and FGF signaling pathways are active and promote growth in a subtype of highly aggressive mCRPC. However, the mechanism for this activation remains elusive. These 5′ UTR mutations could potentially impact the expression levels of critical MAP kinase pathway components. Indeed, orthogonal validation of the FOS and FGF7 5′ UTR mutations in human prostate cancer cells revealed that these single nucleotide alterations identified through PLUMAGE were sufficient to increase gene specific transcript levels (FIG. 4B). Importantly, the increase in mRNA transcript levels was not caused by impaired mRNA degradation due to the mutation or by the different 30-bp barcode used between the WT and mutant constructs (as shown in FIGS. 17A and 17B).

This increase in transcript can be observed in human tissues. Interestingly, the FGF7(chr15: 49715462, C->T) 5′ UTR alteration present in a PDX specimen was associated with a significant increase in FGF7 mRNA transcript abundance by a log2 fold change of 3.09 (FIG. 4C). Furthermore, this specific mutation created a new DNA-binding site element (FIG. 4D). In particular, the somatic alteration from CACGCG to CACGTG created an E-box, the canonical binding motif for the oncogene MYC, which is often deregulated in advanced prostate cancer patients. Importantly, it has been shown that MYC can bind to E-box elements downstream of the transcriptional start site to promote gene specific expression. To verify binding, an electrophoretic mobility shift assay (EMSA) was performed and it was found that the MYC: MAX heterodimer protein complex specifically bound to the E-box sequence created in the mutated FGF7 5′ UTR but did not bind the WT sequence (FIG. 4E). Heterodimer binding was also abolished in the presence of an unlabeled oligonucleotide competitor and when MYC and MAX were tested individually suggesting specific affinity for the E-box sequence created by the FGF7 5′ UTR mutation (FIG. 4E). These findings reaffirm the 5′ UTR as a dynamic region containing regulatory elements that impact transcript levels and demonstrate that creation of neo-promoter elements can affect transcription factor binding and mRNA transcript levels of oncogenic factors.

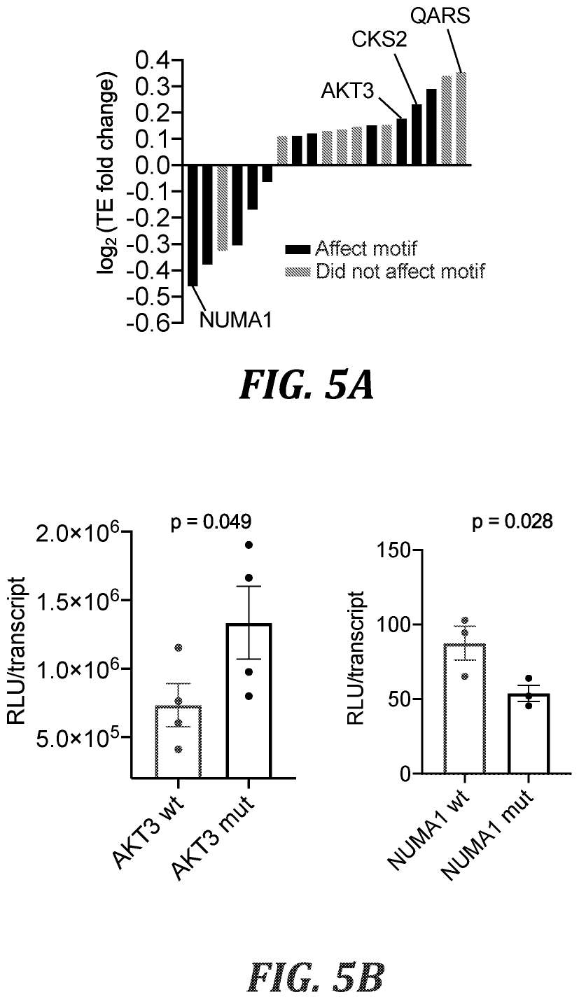

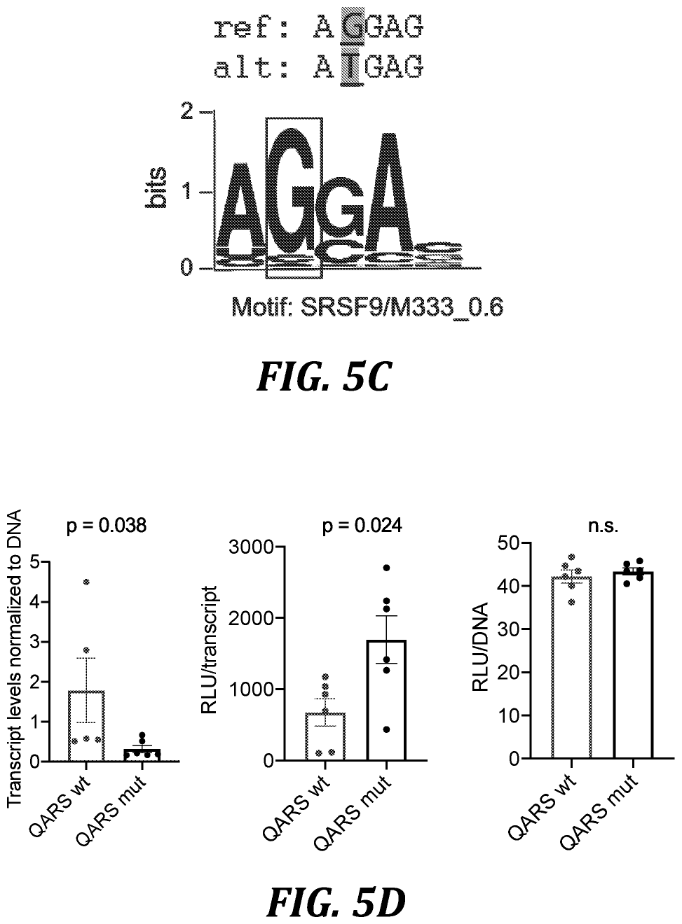

Interestingly, 57.8% of mutations that affected mRNA translation also changed a putative RNA-based cis-regulatory element (FIG. 5A). Such mutations were found in mRNAs that included the oncogene AKT3, the microtubule-binding protein NUMA1, which also has tumor suppressive properties, and the oncogenic cyclin dependent kinases regulatory subunit CKS2.

FIG. 5A shows 5′ UTR mutations that significantly affect mRNA translation efficiency and magnitude fold change compared to unmutated 5′ UTRs, in accordance with the present technology. A proportion of mutations impact known RNA binding protein binding motifs, indicated by black bars (Mann-Whitney U test, FDR<0.1).

FIG. 5B shows validation of 5′ UTR mutations in AKT3 (chr1: 244006547, C->T) and NUMA1 (chr11: 71780891, C->A) by luciferase assay, in accordance with the present technology. Each WT or mutant construct was separately transfected into PC3 cells and assayed for luciferase activity and luciferase mRNA expression after 24 hours. Luciferase activity (RLU) was normalized to the amount of luciferase transcript in each transfection to determine translation efficiency (n=3 to 4 biological replicates, +s.e.m. Student's t-test).

FIG. 5C shows the C->A 5′ UTR mutation in NUMA1 at position chr11, in accordance with the present technology. 71780891 abrogates an existing SRSF9 RNA binding protein motif.

FIG. 5D shows the 5′ UTR mutation in QARS (chr 3: 49142179, G->A) making significant changes in both transcript levels and translation efficiency, not attributable to the amount of DNA transfected, in accordance with the present technology. Each QARS 5′ UTR WT and mutant plasmid was transfected individually into PC3 cells, followed by luciferase assay, luciferase RNA PCR, and luciferase DNA qPCR. RLU values were normalized to luciferase mRNA to determine translation efficiency. Luciferase mRNA was normalized to luciferase DNA to determine the effects on transcript levels (n=5 to 6 biological replicates, +s.e.m. Student's t-test).

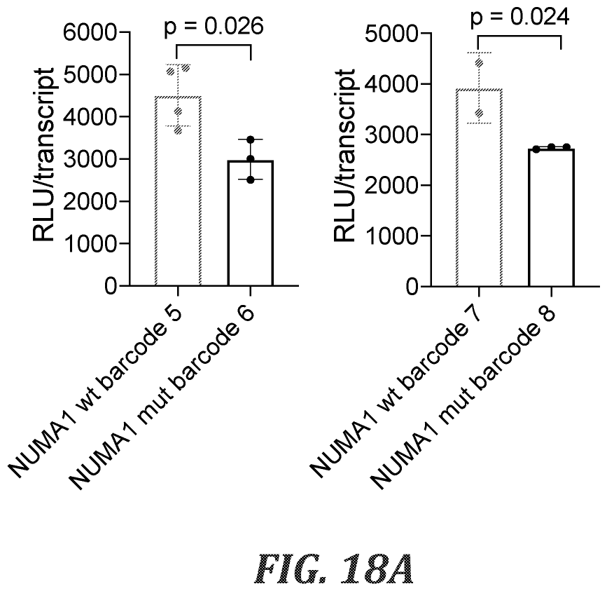

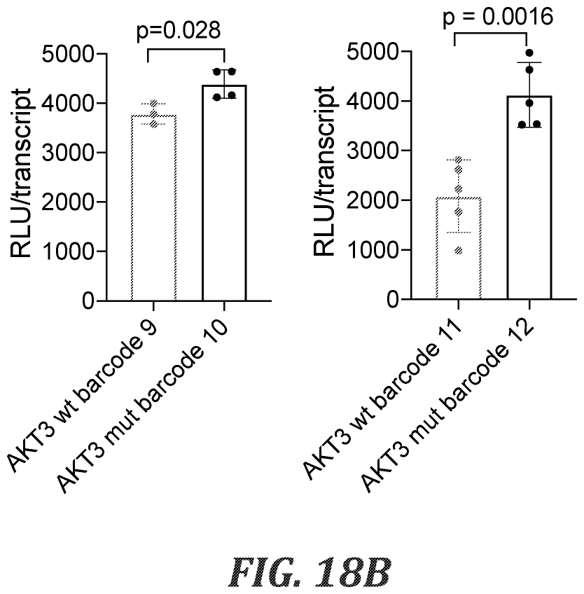

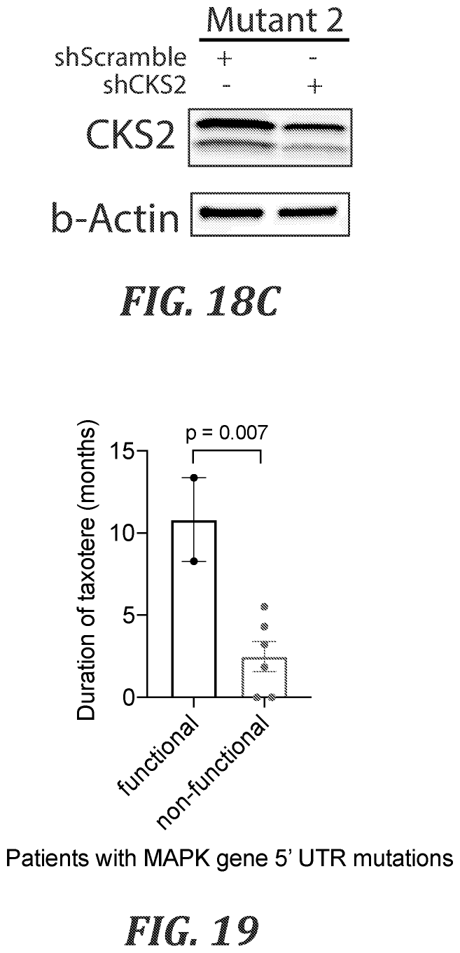

Using luciferase assay normalized by gene specific transcript levels, it was determined the AKT3 mutation (chr1: 244006547, C->T) indeed leads to an increase in protein levels, whereas the NUMA1 mutation (chr11: 71780891, C->A) decreases protein abundance (FIG. 5B). Importantly, the increase and decrease in protein abundance was not affected by the 30-mer barcode encoded in each luciferase construct (as shown in FIGS. 18A and 18B). Interestingly, it was found that the NUMA1 mutation removes a serine and arginine rich splicing factor 9 (SRSF9) RNA binding protein motif found in the Hughes and CISBP RNA binding protein motif datasets (FIG. 5C). SRSF9, which is predicted to interact with this motif, has been implicated in tumorigenesis by deregulating the proper translation of specific mRNAs such as β-catenin. This RNA binding motif appears to be conserved in the serine and arginine-rich protein family; thus, the abrogation of the motif may represent a larger node of gene regulation.

Importantly, one of the features of PLUMAGE is the ability to monitor changes at both the transcript and mRNA translation levels simultaneously. Indeed, it was found that a single point mutation in the 5′ UTR of glutaminyl-tRNA synthetase (QARS) (chr 3: 49142179, G->A) led to a concomitant decrease at the transcript level, but an increase at the level of mRNA translation, which was validated by qPCR and luciferase assay (FIG. 5D). The differences observed were confirmed to not be a result of differential transfection efficiency between the WT and mutant constructs (FIG. 5D). As such, PLUMAGE may uncover 5′ UTR mutations that transcend a single mode of gene expression. Furthermore, these data support the concept that 5′ UTR mutations found in cancer patients within a singular genomic location can dictate the fate of specific genes by co-opting multiple levels of gene expression. Together, these findings demonstrate the significant utility of PLUMAGE in delineating new ways in which somatic mutations within the 5′ UTR can affect the molecular outcome of cancer-associated genes.

It was observed that a C->T mutation in the 5′ UTR of oncogenic CKS2 (chr9: 91926143) creates a new upstream AUG (uAUG) within the 5′ UTR in-frame with the main reading frame.

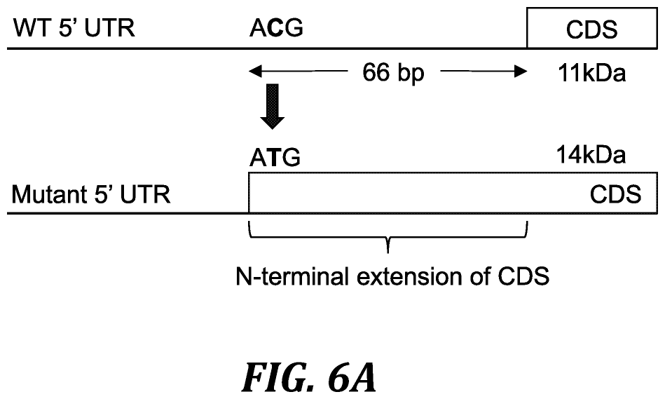

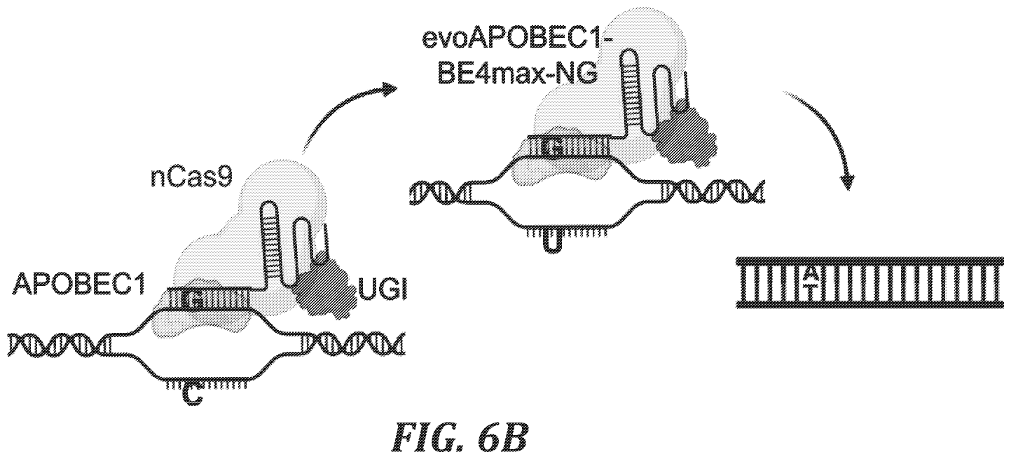

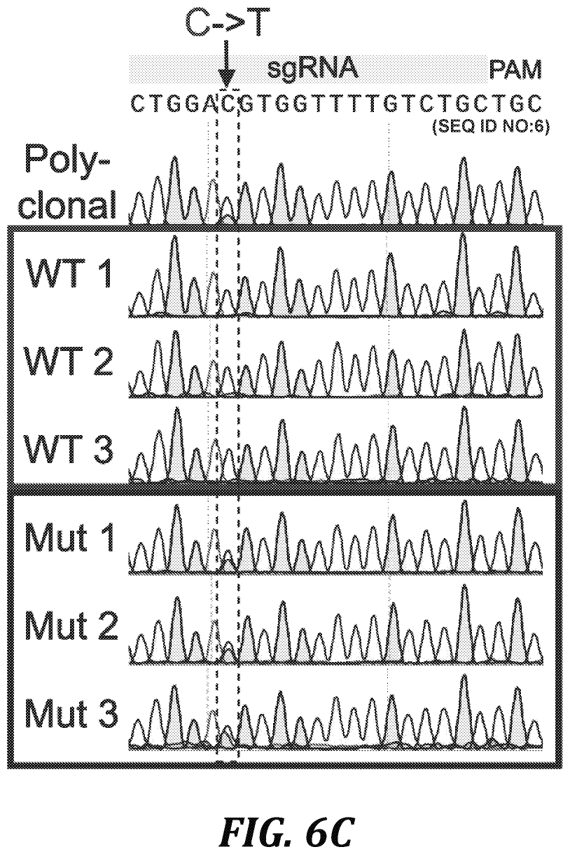

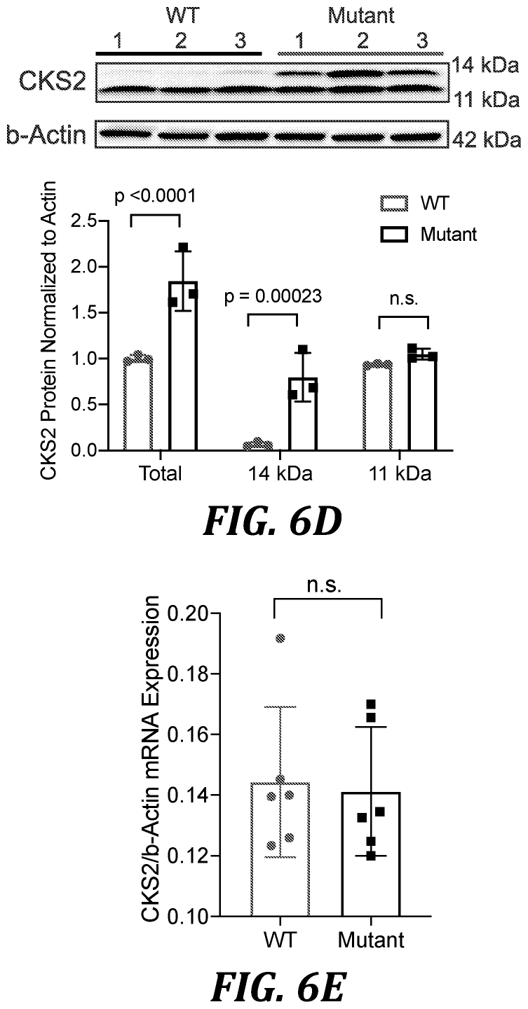

FIG. 6A is a schematic showing wildtype (WT, top) and mutant (bottom) versions of CKS2 transcript, including 5′ UTR, normal coding sequence (CDS), and mutant N-terminally extended CDS, in accordance with the present technology. The C->T mutation (chr9: 91926143) within the 5′ UTR of CKS2 generates a start codon which extends the coding sequence of CKS2.

FIG. 6B is an example method of CRISPR (clustered regularly interspaced short palindromic repeats)-Cas9 (CRISPR-associated protein 9) base editing using evoAPOBEC1-BE4max-NG, in accordance with the present technology. EvoAPOBEC1-BE4max-NG is composed of APOBEC1, a Cas9 nickase domain, and uracil-DNA glycosylase inhibitor (UGI). This base editor deaminates target cytosines to uracil, which changes the original G-C base pair into an A-T base pair after DNA repair. In some embodiments, the method further comprises confirming analyzed target nucleic acid sequences with a process selected from CRISPR-mediated base editing and prime editing, such as with prime editing guide RNA (pegRNA). In this way, it can be determined what the effects of the mutations are in their endogenous context.

FIG. 6C shows Sanger sequencing traces from polyclonal population of CRISPR-transfected 293T cells and 6 individual single-cell clones selected from this pool for further study, in accordance with the present technology. The target C (white)->T (gray) mutation in the 5′ UTR of CKS2 is shown within the dashed box.

FIG. 6D is a western blot of the 3 WT and 3 CKS2 mutant clonal cell lines created by CRISPR base editing with antibodies against CKS2 and beta-actin, in accordance with the present technology. The graph shows these results quantified using ImageJ, where each CKS2 band intensity was measured and normalized to the intensity of the corresponding b-actin loading control. Statistics show student's t-test with multiple comparisons correction using the 3 WT versus 3 CKS2 mutant biological replicates.

FIG. 6E demonstrated that CKS2 qPCR shows no change in mRNA levels between 3 WT and 3 mutant clonal cell lines created from CRISPR base editing, in accordance with the present technology; CKS2 mRNA levels in each sample were normalized to beta-actin as a loading control (n=6 biological replicates).

Interestingly, this uAUG increased overall translation through the CKS2 5′ UTR in PLUMAGE (FIG. 5A). To determine whether this mutation would increase translation of endogenous CKS2, the C->T mutation was engineered using CRISPR cytosine base editing. Nucleotide-specific base editing utilizes a complex of a cytosine deaminase (APOBEC1), a Cas9-nickase, and uracil-DNA glycosylase inhibitor (UGI) (FIG. 6B). The Cas9 domain directs the complex to a target locus, where APOBEC1 deaminates a cytosine to uracil. This results in a G-U base pair that is protected from excision by UGI, which is replaced by cellular mismatch repair to an A-T base pair in place of the original C-G44. Using a sgRNA targeting the CKS2 5′ UTR and the evolved evoAPOBEC1-BE4max-NG base editor 3 CKS2 5′ UTR mutant cell lines were successfully engineered, each with one allele possessing the C->T mutation (FIG. 6C). Next, CKS2 protein levels were measured by western blot analysis and a 2-fold increase in overall CKS2 protein in mutant cells was observed, compared to WT controls (FIG. 6D). This increase was mainly due to expression of the 14 kDa extended CKS2 coding sequence without any noticeable sacrifice of the normal 11 kDa CKS2 coding sequence. Importantly, the specificity of the CKS2 antibody for the 11 kDa and 14 kDa N-terminally extended CKS2 were confirmed by shRNA knockdown (shown in FIG. 18C). Since CKS2 mRNA expression did not differ between the WT and mutant cells (FIG. 6E), it was concluded that the CKS2 chr9: 91926143 C->T mutation was sufficient to increase mRNA-specific translation. This observation corroborates the PLUMAGE findings and demonstrates that 5′ UTR mutations can coordinately impact mRNA translation by altering RNA-based cis-regulatory elements in their endogenous context.

The patient cohort consists of both localized prostate cancer and mCRPC patients, thus enabling the study of the impact of 5′ UTR mutations in early-stage versus advanced metastatic prostate cancer. It was found 5′ UTR mutations that were unique to either localized cancer or mCRPC.

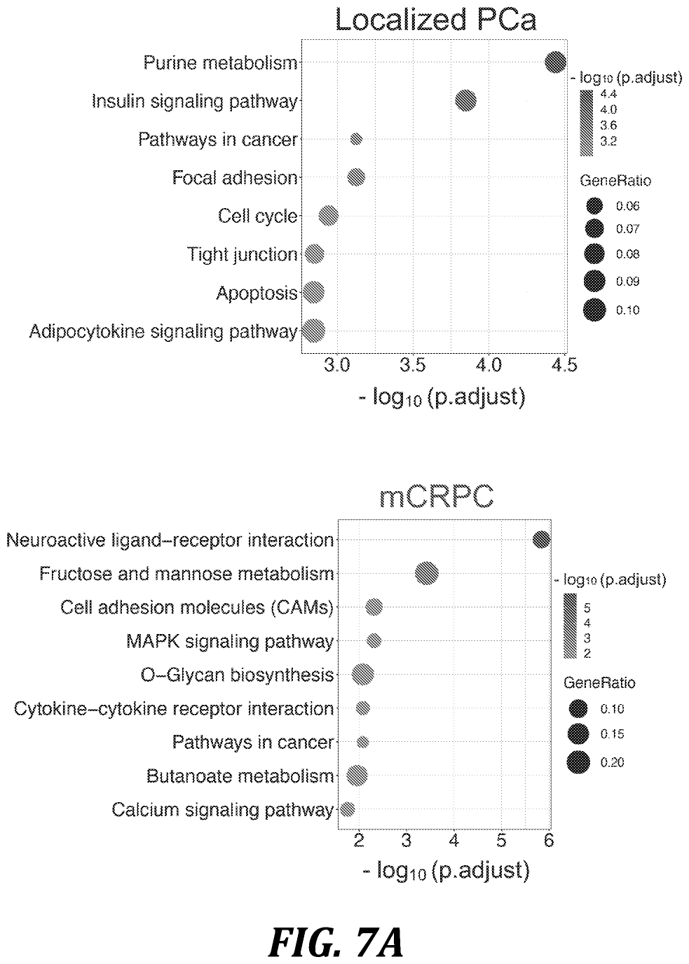

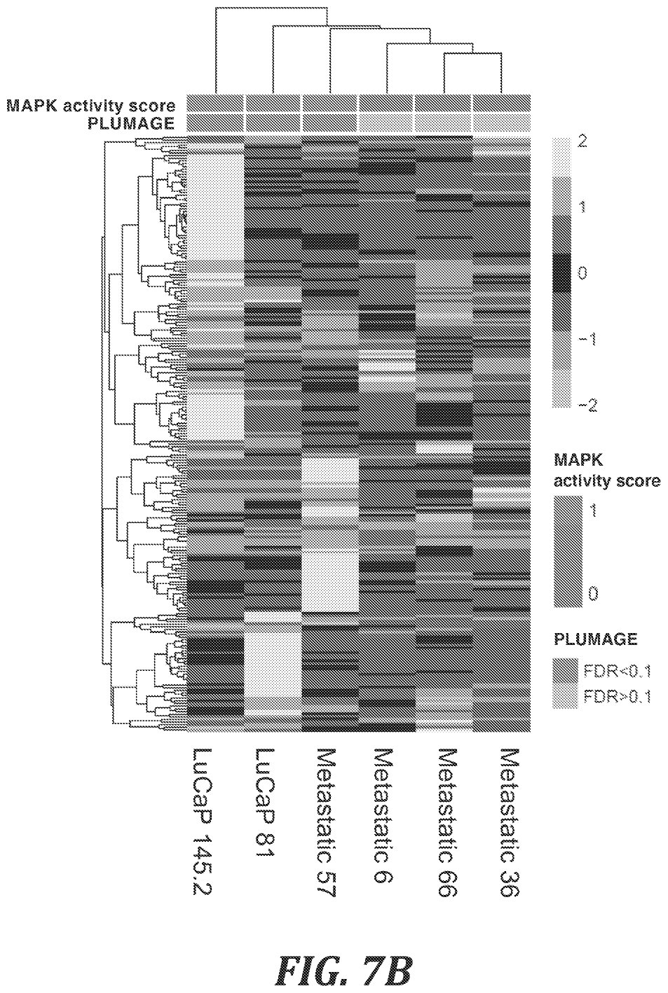

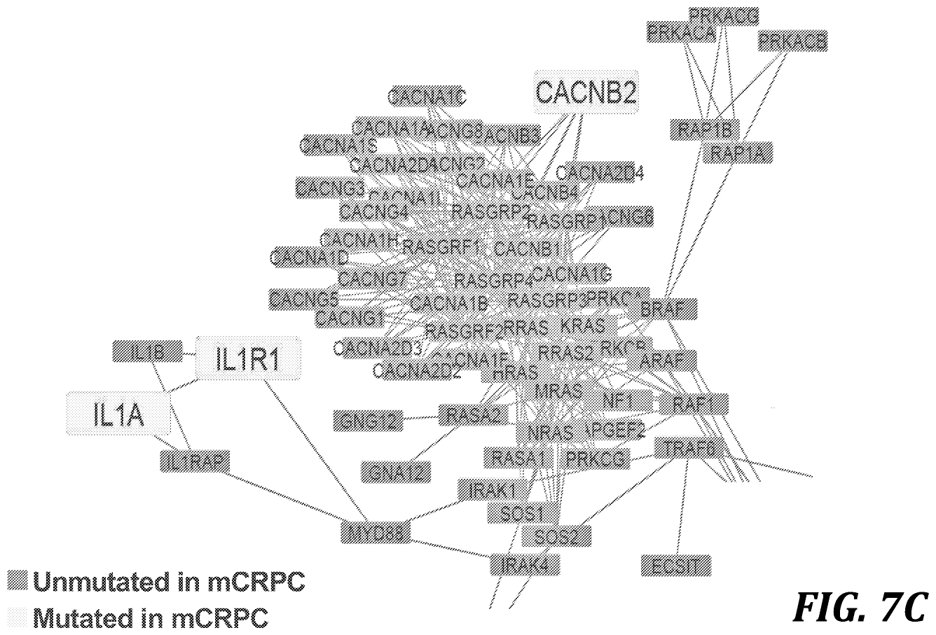

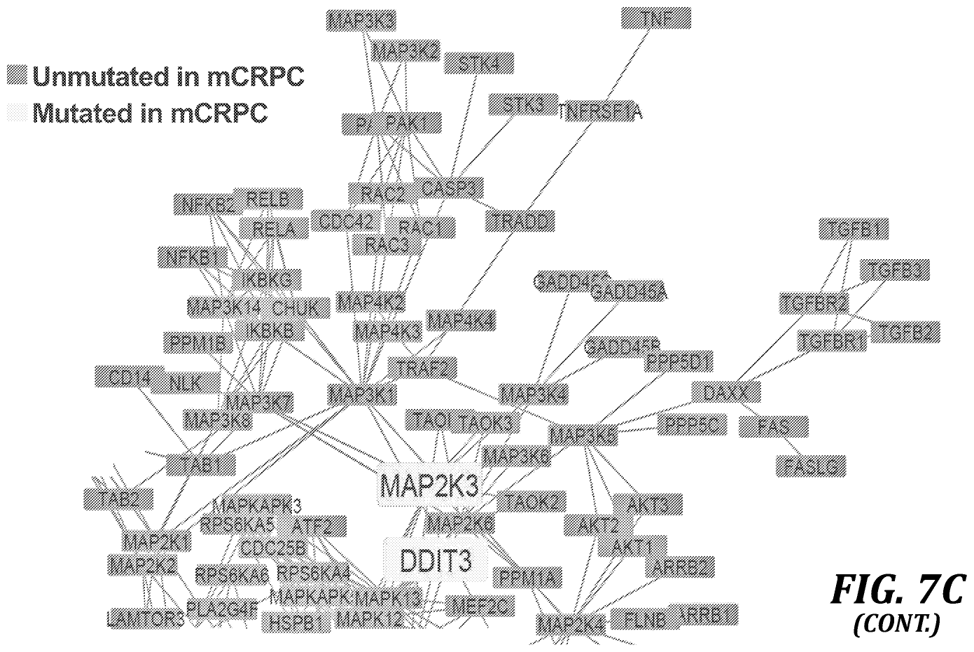

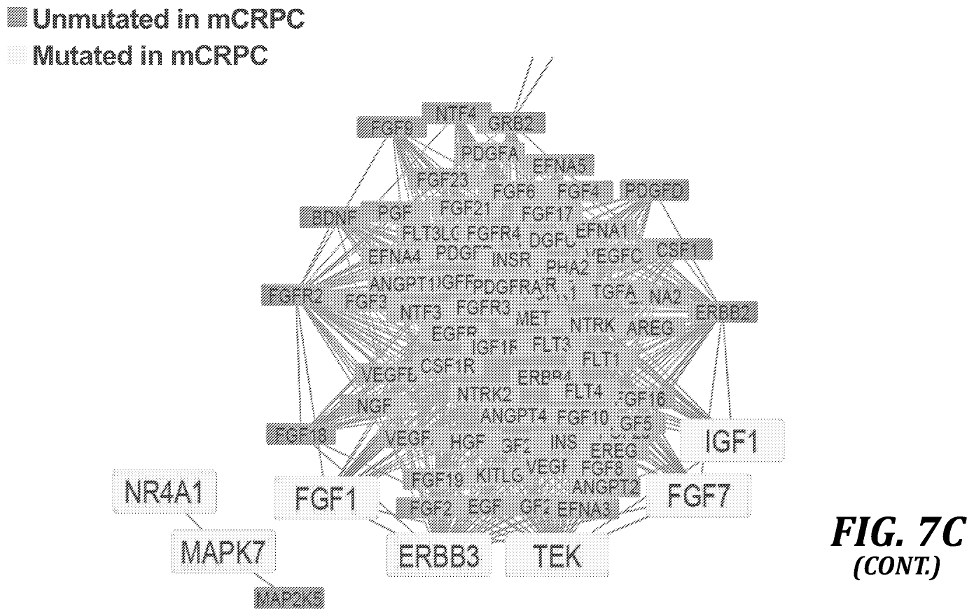

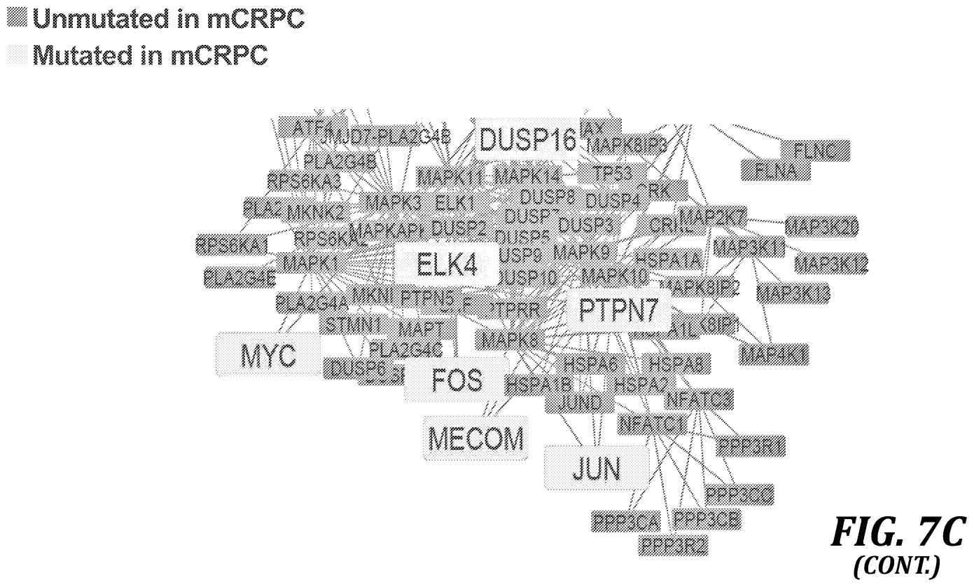

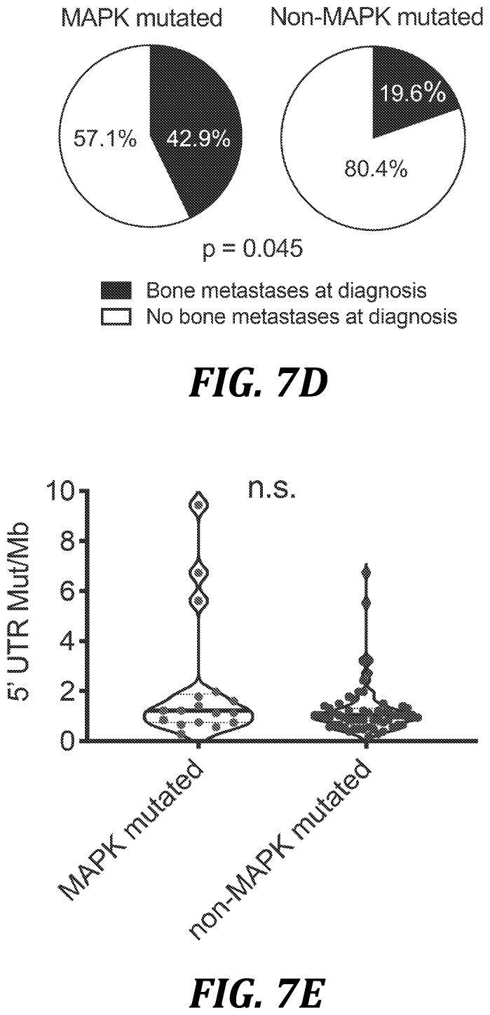

FIG. 7A shows genes with 5′ UTR mutations in localized and advanced prostate cancer cluster into distinct functional categories as determined by KEGG pathway analysis, in accordance with the present technology (FDR<0.05).

FIG. 7B is a heat map of a MAP kinase pathway activity signature demonstrating that patients with functional 5′ UTR mutations to MAP kinase regulators exhibit increased pathway activation compared to non-functional mutations, in accordance with the present technology. The MAP kinase regulators had a PLUMAGE FDR<0.1, and the non-functional mutations had a PLUMAGE FDR>0.1.

FIG. 7C shows metastatic castration resistant prostate cancer patients harbor 5′ UTR mutations within genes found in the MAP kinase signaling pathway, in accordance with the present technology. Gene names in light gray represent those with 5′ UTR mutations in mCRPC patients. Gene names in dark gray are MAP kinase signaling pathway components and downstream effectors that are not mutated in mCRPC patients.

FIG. 7D demonstrates that mCRPC patients with mutated MAP kinase pathway genes are significantly more prone to bone metastases at diagnosis compared to patients who do not harbor these mutations, in accordance with the present technology (p=0.045, Student's t-test).

FIG. 7E shows the difference in bone metastasis at diagnosis between the two patient groups independent of any differences in 5′ UTR tumor mutational burden, in accordance with the present technology (n.s.=not statistically significant, Student's t-test).

Indeed, GSEA analyses showed that 5′ UTR mutations in localized prostate cancer enrich for cell cycle pathways, whereas mutated genes in mCRPC enrich for metabolism and the MAP kinase signaling pathway (FIG. 7A). To determine if functional 5′ UTR mutations of MAP kinase regulators can impact pathway-specific gene expression, RNA sequencing data was analyzed from patients and PDX models harboring MAP kinase pathway mutations tested in PLUMAGE. Interestingly, 3 patients with functional MAP kinase pathway 5′ UTR mutations to FOS, FGF7 and MECOM that were predicted to increase signaling by PLUMAGE demonstrated upregulation of a RAS-driven prostate cancer MAP kinase pathway gene signature (FIG. 7B). Moreover, 3 patients with non-functional 5′ UTR mutations to MAP kinase pathway genes PTPN7, JUN, and DDIT3 did not exhibit changes in gene expression (FIG. 7B). These findings demonstrate that functional 5′ UTR mutations have the ability to impact cancer-associate pathway activity.

Next, functional mutations within 5′ UTRs of MAP kinase pathway regulators were analyzed to determine if they are associated with patient outcomes. Multiple patient endpoints including progression free survival, overall survival, time to metastases, Gleason score, and duration on therapeutic agents were analyzed. Interestingly, it was observed that patients with functional MAP kinase pathway mutations that were predicted to increase signaling by PLUMAGE (FOS and MECOM, FDR<0.1) were more likely to have a sustained response to the microtubule inhibitor and chemotherapy Taxotere compared to patients without functional mutations (as shown in FIG. 19). This observation is consistent with a previous report which demonstrated that taxane-mediated apoptosis is dependent on MAP kinase signaling.

Lastly, 5′ UTR mutations to MAP kinase regulators were analyzed to determine if they could represent a biomarker for disease aggressiveness, because they have been implicated in prostate cancer metastasis. To increase the power of this analysis, all 19 MAP kinase 5′ UTR mutations observed in metastatic patients were analyzed to determine if they correlated with disease physiology (FIG. 7C). Notably, patients harboring mutations in these genes were more likely to present with bone metastasis at diagnosis compared to patients without MAP kinase pathway 5′ UTR mutations (p<0.05) (FIG. 7D). Moreover, this difference was independent of 5′ UTR tumor mutational burden (FIG. 7E). As such, these findings demonstrate the potential clinical association of 5′ UTR alterations in mCRPC patients and highlights the importance of 5′ UTR mutations in oncogenic pathway activation and cancer progression. FIG. 8 shows lengths of all 326 5′ UTRs with somatic mutations in LuCaP PDX samples in accordance with the present technology.

FIG. 9 shows gland enriched normal prostate tissue used for RNAseq and ribosome profiling, in accordance with the present technology.

FIG. 10A shows RNAseq and ribosome profiling of mCRPC PDX tissues, in accordance with the present technology. 5′ UTR somatic mutations, RNASeq and ribosome-bound mRNA reads were obtained from each tissue;

FIGS. 10B-10C are dendrograms of normalized read counts for ribosome-bound and total RNA replicates, in accordance with the present technology.

FIG. 10D shows representative periodicity plots of ribosome-bound mRNA and total mRNA from a PDX issue, in accordance with the present technology; To ensure isolated ribosome-bound mRNAs, sequencing libraries were analyzed for triplet periodicity. For each read length specified, the sum of alignments in the different frames is shown, together with the maximum likelihood frame for ribosome bound (top) and total RNA samples (bottom);

FIG. 10E shows representative periodicity plots showing ribosome bound fragments enriched in one of the three possible codon frames (top) at each base relative to coding start/end, whereas non-protected total mRNA (bottom) is not, in accordance with the present technology.

FIG. 10F shows representative plots of multiple lengths of sequenced reads for ribosome bound (top) and total RNA samples (bottom), in accordance with the present technology. Ribosome footprints around 28-30 bases in length were captured.

FIG. 11A is a scatter plot showing correlation of normalized read counts per 8-bp barcode between biological replicates in small PLUMAGE library, in accordance with the present technology. The validation of PLUMAGE using the luciferase reporter assay is shown (Pearson r=0.91, p=0.0001).

FIG. 11B is a comparison of performance of a construct without Kozak and ATG sequences by PLUMAGE, in accordance with the present technology. As labeled, p=0.014, Student's t-test (n=6 to 8 biological replicates, mean +s.e.m.).

FIG. 11C is a luciferase assay of construct without Kozak and ATG sequences normalized to the amount of luciferase transcript confirms result seen by PLUMAGE, in accordance with the present technology. As labeled, p<0.0001, Student's t-test (n=7 biological replicates, mean +s.e.m.).

FIG. 12A is a schematic diagram of obtaining 5′ UTR somatic mutations from patient samples, in accordance with the present technology. Comparison of mutation rates in 5′ UTRs vs protein coding regions. Publicly available whole-genome sequencing (WGS) data of localized prostate cancer patients were downloaded and analyzed. Genomic DNA was obtained from mCRPC patients, sequenced, and analyzed.

FIG. 12B shows the number of read counts per reference (ref) and altered (alt) nucleotide in the 5′ UTRs of tumor and matched normal samples, in accordance with the present technology. Tumor alt read counts are much higher than normal alt read counts, suggesting the presence of somatic mutations.

FIG. 12C shows the variant allele frequency (VAF) of all 5′ UTR mutations from tumor and matched normal samples show higher tumor VAFs compared to normal tissues, suggesting reliable detection of somatic 5′ UTR mutations, in accordance with the present technology.

FIG. 12D is a comparison of CDS mutation rates between localized prostate cancer and mCRPC patients shows higher mutation rates in mCRPC patients, in accordance with the present technology.

FIG. 12E shows an overlap in a number of genes with mutations in 5′ UTRs and CDS regions, in accordance with the present technology. At least 50% of genes have mutations that are exclusive to either the 5′ UTR or the CDS (***p<0.001, Hypergeometric test).

FIG. 12F shows the number of genes with mutations in 5′ UTRs in the Quigley et al. Cell 2018 dataset compared to the present dataset, in accordance with the present technology (***p<0.001, Hypergeometric test).

FIGS. 13A-13B show the most frequently mutated 5′ UTR regulatory elements in prostate cancer, in accordance with the present technology. FIG. 13A is the most frequently mutated 5′ UTR DNA binding elements in our prostate cancer patient cohort identified using the HOMER database. FIG. 13B is the most frequently mutated RNA binding protein elements in our prostate cancer patient cohort identified using the Hughes database.

FIGS. 14A-14B show determinations of 5′ UTR TSSs and polysome profiling in PLUMAGE experiments, in accordance with the present technology. FIG. 14A shows read counts from RNASeq of mCRPC patients relative to transcription start sites of genes from reference genome (Refseq) from two separate RNASeq datasets. SU2C RNASeq was obtained from publicly available data from Robinson et al. Cell 2015, whereas the UW-TAN RNASeq was obtained from Kumar et al. Nat Med 2016 and were from mCRPC patients we sequenced. All 5′ UTRs assayed in PLUMAGE were compared and had high read counts at the TSS suggesting robust expression in prostate cancer tissue. FIG. 14B shows representative polysome profiling traces from 293T cells and PC3 cells transfected with PLUMAGE plasmid library. The polysome fractions after the disome were pooled (indicated in box) to obtain polysome-bound mRNA for each replicate.

FIGS. 15A-15B show quantification of 30-bp barcodes in PLUMAGE, in accordance with the present technology. FIG. 15A shows the number of unique 30-bp barcodes per mutated and unmutated 5′ UTR sequence in each sample, for each quantitative measurement as determined by taking ratio of total mRNA/DNA and polysome/total mRNA. FIG. 15B shows density plots of normalized read counts per barcode, for each quantitative measurement determined by taking ratio of total mRNA/DNA and polysome/total mRNA. Data shown for all three biological replicates and is representative of both cell lines.

FIGS. 16A-16B show polysome to total RNA measurements of translationally regulated PLUMAGE hits correlating with polysome to 80S measurements, and functional 5′ UTR mutations not associated with regional DNA structural changes, in accordance with the present technology. FIG. 16A is the ratio of polysome-bound mRNA read counts to total mRNA read counts correlates well with ratio of polysome-bound mRNA read counts to 80S-bound read counts in PLUMAGE. Each dot represents a 5′ UTR mutation found to have significant change in translation efficiency by PLUMAGE (FDR<0.1). FIG. 16B is copy number analysis of the regional 5′ UTR genomic structure from patients with functional (FDR<0.01) and non-functional (FDR>0.01) mutations at determined by PLUMAGE (n.s.=not statistically significant, Chi-square test).

FIGS. 17A-17B show FOS and FGF7 5′ UTR mutations increase transcript levels independent of mRNA stability and sequence of the randomer barcode, in accordance with the present technology. FIG. 17A shows the amount of transcript at 0 hr and 1 hr of 10 μM actinomycin D treatment 48 hours after transfection of FGF7 and FOS WT and MUT plasmids. The rate of mRNA degradation between WT and MUT samples do not impact the increase in transcript levels brought about by the mutations (n=3 to 4 biological replicates, mean +s.e.m). FIG. 17B shows FGF7 and FOS 5′ UTR mutations which show increase in transcript levels by qPCR, even with different 30-bp barcodes attached to the 3′ end of the luciferase gene (P=0.024 for FGF7 and p=0.023 for FOS, Student's t-test (n=2 to 4 biological replicates, mean +s.e.m)).

FIGS. 18A-18C show different randomer 30-bp barcode used in PLUMAGE that do not impact translation efficiency differences, in accordance with the present technology. FIG. 18A shows different 30-bp barcodes in NUMA1 WT and mutant plasmids do not affect the decrease in translation efficiency as a result of the mutation (C->A, chr11: 71780891). The data shows 4 different barcodes (barcodes 5, 6, 7, 8) performed in biological replicates (n=2 to 4, mean +s.d.). P values were calculated using the Student's t test. FIG. 18B shows different 30-bp barcodes in AKT3 WT and mutant plasmids do not affect the increase in translation efficiency as a result of the mutation (C->T, chr1: 244006547). The data shows 4 different barcodes (barcodes 9, 10, 11, 12), performed in biological replicates (n=3 to 5, mean +s.d.). P values were calculated using the Student's t test. (18C) Immunoblot of CKS2 5′ UTR knock-in mutant cell line after shRNA knockdown of CKS2 demonstrates the specificity of the antibody and

FIG. 19 shows that patients with MAP kinase pathway gene mutations that significantly alter gene expression by PLUMAGE were more sensitive to Taxotere therapy, in accordance with the present technology. Comparison of patients with functional MAP kinase regulator hits by PLUMAGE (FDR<0.1) versus patients with non-functional MAP kinase pathway 5′ UTR mutations (FDR>0.1) are shown. P value was calculated using the Student's t-test (n=2 to 6 patients, mean +s.e.m.).

### EXAMPLES

### Patient Enrollment and Tissue Acquisition

The Institutional Review Board (IRB) of the University of Washington and the Fred Hutchinson Cancer Research Center approved all procedures involving human subjects. Tissue samples were obtained from male patients enrolled in the Prostate Cancer Donor Program at the University of Washington, who died of metastatic castration resistant prostate cancer. All patients in the study signed written informed consent for a rapid autopsy performed within 6 hours of death. All tissues were assessed and acquired as previously described. 80 metastatic tumor samples and their corresponding matched normal tissue were obtained from individual patients. Normal prostate tissue of high glandularity were also obtained from five individuals, as shown in FIG. 9.

### Patient Derived Xenograft Tissue

The five LuCaP series of prostate cancer xenografts used in this study (LuCaPs 78, 81, 92, 145.2 and 147) were obtained from the University of Washington Prostate Cancer Biorepository and generated from advanced prostate cancer patients.

### Cell Lines

Human embryonic kidney 293T (HEK 293T) cells obtained from ATCC were cultured in Dulbecco's modified Eagle's medium (Gibco) supplemented with 10% fetal bovine serum (FBS) and 1% penicillin and streptomycin. The human prostatic carcinoma cell line PC3 obtained from ATCC was cultured in RPMI 1640 medium (Gibco) supplemented with 10% FBS and 1% penicillin and streptomycin. Cells were grown at 37° C. in a humidified atmosphere containing 5% CO₂. 0.05% Trypsin-EDTA solution (Gibco) was used to detach cells from culture dishes. The cell cultures for HEK 293T and PC3 both tested negative for the presence of mycoplasma and were authenticated by short tandem repeat profiling and matched to STR profiles from the ATCC database for human cell lines.

### Genomic UTR Sequencing

Genomic DNA from frozen tissue was extracted using the Qiagen Gentra Puregene Tissue Kit (Qiagen). Sequencing libraries were prepped with the KAPA HyperPrep kit (Roche) using 1 μg of DNA. DNA was sheared using a Covaris LE220 ultrasonicator targeting 200 bp, and sequencing adaptors added by ligation. Individually barcoded libraries were pooled 4-plex before capture. Libraries were hybridized to SeqCap EZ Choice probes of the 50 Mb Human UTR Design (Roche), and sequenced on a HiSeq 2500 (Illumina) using a PE100 in high-output mode. Image analysis and base calling were performed using Illumina's Real Time Analysis v1.18.66.3 software, followed by demultiplexing of indexed reads and generation of FASTQ files, using Illumina's bcl2fastq Conversion Software v1.8.4.

### Ribosome Profiling

Flash frozen human tumors dissected from each LuCaP PDX were manually pulverized under liquid nitrogen and lysed in 1 ml mammalian lysis buffer according to the TruSeq Ribo Profile

(Mammalian) protocol (Illumina). Normal human prostate tissues from high glandular areas were obtained in the form of frozen shavings (200 mg) and lysed in lysis buffer. To impede post-lysis translation, the lysis buffer was supplemented with cycloheximide (Sigma) dissolved in EtOH, at a final concentration of 0.1 mg/ml. For complete tissue lysis, the samples were further mechanically dissociated using a gentleMACS™ Dissociator (Miltenyi Biotec). Lysates were centrifuged, and the supernatants were used to isolate both total RNA and ribosome bound fractions using the TruSeq Ribo Profile (Mammalian) kit (Illumina). Ribosomal RNA was removed using the RiboZero Gold Magnetic Kit (Epicentre) before polyacrylamide gel electrophoresis (PAGE) purification. Ribosome footprints were generated by treating a portion of the lysate with 0.5 μL of TruSeq Ribo Profile nuclease per sample for 45 minutes at room temperature. Resulting monosomes were purified using sephacryl S400 columns (GE Healthcare), from which ribosome protected mRNA fragments were isolated and used to prepare ribosome footprint libraries. All libraries were quantified using the Qubit 2.0 fluorometer (Invitrogen), while the quality and average fragment sizes were estimated using a Bioanalyzer (High Sensitivity assay, Agilent). Barcodes were used to perform multiplex sequencing and create sequencing pools containing multiple samples with equal amounts of both total mRNA and ribosome footprints. The pools were sequenced on the HiSeq 2500 platform using SR50 sequencing chemistry.

### Luciferase Assays

To generate constructs for use in the luciferase reporter gene assay, primers containing Nco1 and HindIII restriction enzyme sites were used to PCR amplify both the wild-type and mutant 5′ UTRs from cDNA generated from the patient derived xenografts, using the Phusion HiFi mastermix (ThermoFisher). These PCR products were purified by gel excision, digested with the Nco1 and HindIII restriction enzymes (NEB), and cloned into the linearized pGL3-promoter-luciferase vector (Promega) using Quick Ligase (NEB) according to manufacturer's protocol. The ligated product was transformed into chemically competent E. coli, plated onto LB agar plates containing ampicillin. Single bacteria colonies were inoculated into LB and grown overnight at 37° C. Plasmid DNA was extracted from the bacteria cultures using the QIAprep mini kit (Qiagen), and Sanger sequenced to verify the cloned product. The successfully cloned plasmids containing the wild-type and mutant 5′ UTR sequences of interest were transfected into cell lines using Lipofectamine 3000 (Invitrogen) according to the manufacturer's protocol. Firefly luciferase activity was measured 24 hours after transfection using the Dual-Glo Luciferase assay system (Promega) according to the manufacturer's instructions. Luminescence was measured on a BioTek Synergy HT (BioTek), and data were collected via the Gen5 2.01.14 software. Relative luminescence units (RLU) from the luciferase assays were normalized against the amount of luciferase transcript by qPCR, as a quantitative read out of translation efficiency. Box plots show lines at median, 25th and 75th percentiles. Error bars reflect minimum and maximum values.

### Quantitative PCR (qPCR)

To validate changes in transcript levels brought about by 5′ UTR mutations, RNA and DNA were extracted from PC3 cells transfected with individual FOS, FGF7 and QARS WT and mutant plasmids using the AllPrep DNA/RNA Mini Kit (Qiagen). cDNA synthesis was performed on 1 μg of RNA using the SuperScript First Strand Synthesis System (Invitrogen) and a RT primer. qPCR was performed on the DNA and cDNA using SsoAdvanced Universal SYBR Green Supermix (BioRad) in triplicates, with primers against luciferase (For: GTGTTGGGCGCGTTATTTATC (SEQ ID NO. 6), Rev: TAGGCTGCGAAATGTTCATACT (SEQ ID NO. 7)). To validate changes in mRNA translation, RNA and luciferase activity were collected from PC3 cells transfected with individual NUMA1, AKT3 and QARS WT and mutant plasmids. Total mRNA was extracted using the Quick-RNA Miniprep Plus kit (Zymo Research), and cDNA synthesis and qPCR was performed as described. For the CKS2 experiment RNA was extracted from ˜500,000 cells per 293T CKS2 WT or Mutant cell line using the RNeasy Plus kit (Qiagen) following the manufacturer's protocol. cDNA was synthesized using 500 ng RNA and iScript RT Supermix (BioRad) or iScript NRT Supermix for negative controls. qPCR was performed using SsoAdvanced Universal SYBR Green Supermix (BioRad) on 1 μL of each cDNA, NRT, and NTC sample in triplicate using primers specific to CKS2 (For: CACTACGAGTACCGGCATGTT (SEQ ID NO. 8), Rev: ACCAAGTCTCCTCCACTCCT (SEQ ID NO. 9)) and β-actin (For: AAATCTGGCACCACACCTTC (SEQ ID NO. 10), Rev: GGGGTGTTGAAGGTCTCAAA (SEQ ID NO. 11)) as a housekeeping control.

### Construction of Master pGL3 Reporter Backbone for PLUMAGE

The pGL3-promoter-luciferase plasmid (Promega) was linearized using the Xba1 restriction enzyme (NEB). A 202-bp double-stranded DNA fragment (IDT) containing an EcoRI restriction enzyme site followed by a 36-bp spacer sequence was cloned into the pGL3-promoter vector by Gibson assembly using the Gibson assembly mastermix (NEB) (Sequence of 202-bp double-stranded DNA fragment: AAGTACCGAAAGGTCTTACCGGAAAACTCGACGCAAGAAAAATCAGAGAGATC CTCATAAAGGCCAAGAAGGGCGGAAAGATCGCCGTGTAATtctagagaattctcatgtaattagt tatgtcacgcagatcggaagagcGTCGGGGCGGCCGGCCGCTTCGAGCAGACATGATAAGAT ACATTGATGAGTTTGGACAAAC (SEQ ID NO. 12)). Successfully assembled plasmids were verified by Sanger sequencing. This master luciferase reporter backbone was then digested with both the HindIII and EcoRI restriction enzymes (NEB) according to the manufacturer's instructions, and the larger fragment was gel excised, purified and used as the backbone for cloning the PLUMAGE library.

### Generation of Randomer Barcoded Master pGL3 Reporter Backbone

Barcoded DNA fragments containing the luciferase gene were generated by PCR, using the pGL3-promoter master reporter described above containing EcoRI and spacer sequences as a PCR template. An 80-bp oligonucleotide encompassing a semi-random 30-bp barcode sequence (15 repeats of A/T (W)-G/C(S)) was synthesized by IDT, and used as a reverse primer in the PCR reaction, along with a universal forward primer with sequences corresponding to the beginning of the luciferase gene. The PCR reaction was performed for 15 cycles, in 96-well plates, using the Phusion high-fidelity polymerase with HF buffer (ThermoFisher). Following the PCR reaction, 1 μL of Dpn1 (NEB) was added to each well, along with Cutsmart buffer (NEB), and incubated at 37° C. for 45 minutes to digest the PCR template. A 96-well format DNA cleanup and concentrator kit (Zymo Research) was used to purify the PCR reaction in each well, according to manufacturer's instructions. Each reaction was eluted in 21 μL of elution buffer. A total of ten 96-well plates of barcoded luciferase PCR products were generated.

### Full-length 5′ UTR DNA Sequences

A total of 914 full-length wild-type and mutant 5′ UTR sequences from 329 genes mutated in 2 or more patients or comprising oncogenic lesions were synthesized as double-stranded DNA fragments (IDT and SGI-DNA). Given the variability of transcription start sites (TSSs), putative TSSs of all 5′ UTRs assayed were confirmed by comparing the reference TSS (Refseq) with cumulative 5′ UTR reads of each gene across two independent prostate cancer RNASeq datasets. Each fragment was flanked with 36 bp of homology sequences for Gibson assembly. The homology sequence GAGGAGGCTTTTTTGGAGGCCTAGGCTTTTGCAAAA (SEQ. ID NO. 13) was added to the 5′ end of each 5′ UTR sequence, while the other homology sequence CATGGAAGACGCCAAAAACATAAAGAAAGGCCCGGC (SEQ. ID NO. 14) was added to the 3′ end of each 5′ UTR sequence. 69 out of 329 genes (20%) required small modification to allow for synthesis. These small modifications involve removal of repeat sequences and were completed for matched wild-type and mutant pairs.

### Cloning of PLUMAGE Libraries

Full-length 5′ UTR sequences and barcoded luciferase PCR products were cloned into the pGL3-promoter master reporter backbone using the Gibson Assembly HiFi 1-Step kit (SGI-DNA). Each cloning reaction was carried out in each well in a 96-well plate, and consisted of 1 μL of barcoded PCR product, 1 μL of linearized master reporter backbone, 3 μL of 5′ UTR DNA fragment, and 5 μL of Gibson Assembly 1-step mastermix. For 5′ UTR sequences greater than 1000 bp in length, 2 μL of DNA fragment and 2 μL of barcoded PCR product was used. The reaction was incubated at 50° C. for 1 hour, after which 1.5 μL was transformed into 20 μL of 5-alpha chemically competent E. coli in 96-well plates (NEB) and transformed according to the manufacturer's protocol. 180 μL of room temperature SOC was added to each well and incubated at 37° C. for 90 minutes. The SOC transformants in each well were pooled from each 96-well plate, and 2 mL was plated onto a 500 cm² LB agar plate containing ampicillin at a final concentration of 100 μg/mL. 3 agar plates were used per 96-well plate to generate sufficient numbers of colonies to adequately represent each 96-well plate. To constrain the library size, approximately 300 bacteria colonies per well (or ˜30,000 colonies per 96-well plate) were collected. Plasmid DNA was subsequently extracted using the Endotoxin-free Maxiprep Kit (Qiagen). The plasmid DNA concentration from each maxiprep was measured using the Qubit dsDNA HS assay (ThermoFisher) and pooled in equimolar amounts to form a plasmid DNA library that consist of approximately 300,000 unique barcodes.

### SMRT Sequencing of Plasmid DNA Library

To verify the identity of each wild-type and mutant 5′ UTR, and to simultaneously associate it with unique 30-bp barcode sequences, the pooled plasmid DNA library was sequenced using long-read PacBio Sequel v3.0 sequencing chemistry (Pacific Biosciences). The plasmid DNA library was first linearized using the Sal1 restriction enzyme (NEB), which resides downstream of the 30-bp barcode. Since certain 5′ UTRs also harbor the Sal1 recognition sequence (GTCGAC), and will be truncated, given the restriction enzyme sequence can be found in genomic sequences, these were re-transformed into bacteria, harvested in a separate pool with approximately 300 bacterial colonies per transformation, DNA purified, and linearized with the BamH1 restriction enzyme (NEB). Linearized plasmids from both pools ranging from 5000 bp to 7500 bp were size selected and eluted using the BluePippin system (Sage Science). DNA quantity of the eluates was measured for each pool (Sal1 and BamH1-generated pools) using an Agilent 4200 TapeStation, and 500 ng from each pool was used to prepare a SMRTbell library. Prior to ligation of the hairpin adapters that bind the sequencing primer and DNA polymerase, amplicons underwent damage-and-end-repair to create double-stranded amplicon fragments with blunt ends. The resulting SMRTbell libraries were purified with PacBio AMPure PB beads, combined with a sequencing primer and polymerase, and loaded onto the SMRT cell. The Sal1-generated pool was sequenced over three SMRT cells, while the BamH1-generated pool was sequenced over one SMRT cell.

### Construction of Small Proof-of-concept PLUMAGE DNA Library

This small library was constructed using a different cloning strategy, by utilizing a fixed number of known 8-bp barcode sequences. Luciferase plasmids containing full-length unmutated and mutated 5′ UTR sequences of ADAM32, COMT and ZCCHC7 were linearized, and the 8-bp barcode was cloned at the end of the luciferase coding sequence by PCR. Each barcode was cloned in a separate cloning reaction, transformed into chemically competent E. coli and sequenced to determine successful assembly. Each plasmid with its unique 8-bp barcode was pooled in equimolar amount and transfected into PC3 cells. Long and short-read sequencing were performed as described above. Box plots show lines at median, 25th and 75th percentiles. Error bars reflect minimum and maximum values.

### Transfection of DNA Library Into 293T and PC3 Cell Lines

2.6×106 293T cells were plated onto a 15 cm dish, incubated overnight, and transfected with 16 μg of plasmid DNA library using Lipofectamine 3000 reagent (Invitrogen) according to manufacturer's protocol. 24 hours after transfection, cells were washed with PBS, harvested with 0.05% Trypsin-EDTA (Gibco) and centrifuged at 300×g for 5 minutes into a cell pellet. For the PC3 cell line, 3×106 cells were plated onto a 15 cm dish and transfected with 16 μg of plasmid DNA library using Lipofectamine 3000 reagent (Invitrogen) according to manufacturer's protocol. 24 hours after transfection, cells were washed with PBS, harvested with 0.05% Trypsin-EDTA (Gibco) and centrifuged at 300×g for 5 minutes into a cell pellet. 2.6×106−3×106 cells/plate were chosen to enable over 900× coverage of the plasmid library per replicate (assuming 100 plasmids/cell>25% transfection efficiency, and 212,325 unique constructs within the library). In both cell lines, cell pellets collected from each 15 cm dish were resuspended in 1 mL of cold PBS (Gibco) +100 μg cycloheximide (Sigma) and incubated on ice for 10 minutes. The cells were centrifuged into a cell pellet and lysed in 220 μL of lysis buffer (Tris-HCl, NaCl, MgCl₂, 10% NP-40, Triton-X 100, SUPERase In RNase Inhibitor, cycloheximide, DTT, DEPC water) for 45 minutes on ice, and vortexed every 10 minutes. For each cell line, lysates from three 15 cm dishes were pooled together to form one biological replicate. A total of three biological replicates were performed for each cell line. From each replicate, 60 μL of lysate was collected for DNA extraction using the QIAprep Spin Miniprep Kit (Qiagen). To collect total mRNA, 800 μL of Trizol (Life Technologies) was added to 150 μL of lysate and stored at −80° C.

### Polysome Profiling

The remaining lysate from each biological replicate were centrifuged at 10,000 rpm for 5 minutes at 4° C. to pellet cell debris, and the supernatants were transferred into fresh tubes. 350 μL of the supernatant was layered onto 10% to 50% (w/v) sucrose gradients for ribosome fractionation. The gradients were centrifuged at 37,000 rpm for 2.5 hrs at 4° C. in a Beckman SW41Ti rotor and fractionated by upward displacement into collection tubes through a Bio-Rad EM-1 UV monitor (Biorad) for continuous measurement of the absorbance at 254 nm using a Biocomp Gradient Station (Biocomp). 80S and polysome samples were collected and subsequently processed for sequencing. In particular, polysome fractions (3 or more ribosomes) were pooled; RNA extracted from this pool was compared to total mRNA to determine translation efficiency. Additionally, the pool of polysome fractions was also compared to 80S bound mRNA as an alternate measure of translation.

### RNA Extraction and cDNA Synthesis

Total, 80S-associated, and polysome-bound RNA were extracted using the Direct-zol RNA Miniprep Plus kit (Zymo Research) following the manufacturer's protocol including the on-column DNase digestion. For polysome, RNA samples after the disome were pooled before RNA extraction. To ensure that there was no plasmid carryover, and that mRNA expression in the assay was truly being detected, an additional DNase treatment was performed on 2 μg of extracted RNA using 3 μL of DNasel Amplification grade (Invitrogen) in a total reaction volume of 20 μL, at room temperature for 30 minutes. The reaction was terminated by the addition of 2 μL of 25 mM EDTA with a 10-minute incubation at 65° C. Of this DNase-treated RNA, 8 μL was used in a cDNA synthesis reaction using the SuperScript III First-Strand Synthesis System (Invitrogen) with a primer specific to the 3′ end of the 30-bp barcode. Sequence of gene-specific primer used for first-strand cDNA synthesis: acactctttccctacacgacgctcttccgatctgcgtgacataactaattacatga (SEQ. ID NO. 15). Negative control reactions without the SuperScript III reverse transcriptase enzyme were also performed on all the RNA samples and confirmed to be negative. Reactions were incubated according to manufacturer's instructions.

### Illumina Sequencing Library Preparation

Sequencing libraries were generated by performing 1st and 2nd round PCRs on each DNA, and cDNA generated from total, 80S-associated, and polysome-bound RNA samples. 1st round PCR primers contain target-specific sequences flanking the 30-bp randomer barcode and Illumina adaptor sequences, producing a product of 215 bp. The 1st round PCR reaction was performed using 2× Phusion Flash Mastermix (ThermoFisher) in a 50 μL reaction. The PCR reaction consisted of 5 μL of DNA or cDNA template, 2 μL of forward primer (10 μM), 2 μL of reverse primer (10 μM) and 25 μL of Phusion Flash Mastermix. Thermal cycling conditions were at 95° C. for 3 min, 20 cycles of (98° C. for 10 sec, 60° C. for 30 sec, 72° C. for 30 sec), followed by 72° C. at 5 min. A small portion (3 μL) of the PCR products and negative controls were run on a 1.5% agarose gel for visual inspection. The 1st round PCR products were purified using a 0.8× AMPure XP (Beckman Coulter) cleanup following the manufacturer's protocol with 80% ethanol. Following cleanup, 4 μL of the purified 1st round PCR product was used as a template in the 2nd round PCR reaction. The forward primer contained the Illumina adaptor sequence, as well as the flow cell attachment sequence, the reverse primer contained an 8-bp index between the adaptor sequence and flow cell attachment sequence. The 2nd round PCR reaction was carried out in a 50 μL reaction similarly, using Phusion Flash Mastermix (ThermoFisher), with 5 μL of each forward and reverse primer (0.5 μM). Thermal cycling conditions were at 95° C. for 3 min, 8 cycles of (95° C. for 30 sec, 55° C. for 30 sec, 72° C. for 30 sec), followed by 72° C. at 5 min. PCR products were purified using a 0.8× AMPure XP (Beckman Coulter) cleanup following the manufacturer's protocol with 80% ethanol. A sample (3 μL) of the purified PCR products were run on a 1.5% agarose gel for visual inspection. Each sample was quantified by qPCR using the KAPA Library Universal Quantification kit (KAPA Biosystems) according to the manufacturer's instructions and pooled in equimolar amounts for multiplex sequencing. The final pool was denatured and diluted to a loading concentration of 7.5 pM as per Illumina protocol. The PhiX control library (Illumina) was spiked in at 20% to add diversity for improved cluster imaging.

The libraries were sequenced employing a paired-end, 100 base read length (PE100) sequencing strategy on a HiSeq 2500 (Illumina). Image analysis and base calling were performed using Illumina's Real Time Analysis v1.18.66.3 software, followed by demultiplexing of indexed reads and generation of FASTQ files, using Illumina's bcl2fastq Conversion Software v1.8.4.

### Electrophoretic Mobility Shift Assay (EMSA)

MYC and MAX were translated individually or together in vitro using the TnT SP6 coupled wheat germ extract system (Promega), according to manufacturer's protocol. Plasmids used for MYC and MAX were pCS2-FLAG-hMYC and pRK7-HA-hMAX respectively and were generously provided by the Eisenman Lab (Fred Hutchinson Cancer Research Center). The protein concentrations of the in vitro translated products were determined using the Pierce BCA protein assay kit (ThermoFisher Scientific). Binding reactions were carried out using Odyssey EMSA buffer kit (LI-COR), where 90-100 μg of the translated proteins were incubated with 7.5 nM IRDdye 700-labeled FGF7 WT or mutant DNA probes (IDT) in the presence or absence of their respective unlabeled competitor oligos (IDT), according to manufacturer's protocol. To separate the DNA-protein complex, the binding reactions were subjected to electrophoresis on a 6% DNA retardation gel (ThermoFisher Scientific), which was then scanned using the Odyssey infrared imaging system (LI-COR) to detect the fluorescence signal. The assay was performed three times and showed similar results.

### Actinomycin D RNA Degradation Study

10 μM of actinomycin D (Sigma, prepared in DMSO), or an equivalent volume of DMSO (Gibco) as a control, was added to PC3 cells in culture 48 hours after transfection with WT or mutant plasmids. Cells were harvested prior to actinomycin D treatment, and again after 1 hour of treatment. RNA was extracted for cDNA synthesis and subsequent qPCR amplification and quantitation of luciferase mRNA expression.

### CRISPR Base Editing

Plasmid to express CKS2-targeting sgRNA was cloned using the Q5 site-directed mutagenesis kit (NEB) according to manufacturer's instructions. The pFYF1320 sgRNA expression plasmid was used as a template for Q5 mutagenesis PCR (For: TTTTGTCTGCGTTTTAGAGCTAGAAATAGCAAG (SEQ. ID NO. 16), Rev:
CCACGTCCAGGGTGTTTCGTCCTTTCCAC (SEQ. ID NO. 17)) to replace the existing sgRNA sequence with the CKS2-targeting sgRNA sequence (CTGGACGTGGTTTTGTCTGC (SEQ. ID. NO. 18)).

293T cells were plated in 6-well plates at 375,000 cells/well, incubated at 37° C. overnight, and transfected with 1,125 ng evoAPOBEC1-BE4max-NG (Addgene: 125616), 375 ng CKS2 sgRNA expression plasmid, and 30 ng pMaxGFP using Fugene HD (Promega) according to manufacturer's protocol. 72 hours post-transfection, cells were washed with PBS, harvested with 0.05% Trypsin-EDTA (Gibco), and centrifuged at 400× g for 5 minutes. This cell pellet was resuspended in PBS and sorted using flow cytometry for live, singlet, GFP+ cells on a Sony SH800 sorter. GFP+ cells were plated using limiting dilution in 10 cm plates to grow out single-cell clones. After clones had grown sufficiently (˜3 weeks), DNA was extracted using Zymo's MicroPrep Quick-DNA kit, the CKS2 locus PCR amplified using the Phusion High Fidelity Mastermix (ThermoFisher) in a 25 μL reaction and primers: (Forward primer: ACTTCCGCAGAAGGTGATTG (SEQ. ID NO. 19), Reverse primer: TACTCGTAGTGTTCGTCGAAGT (SEQ. ID NO. 20)), according to manufacturer's protocol. PCR products were then Sanger sequenced to determine if the intended CKS2 mutation (chr9: 91926143 C->T) had been introduced. Six individual clonal cell lines were chosen for further testing: 3 mutant clones each mutated at 1 of 2 CKS2 alleles, and 3 WT clones that were not mutated.

### shRNA Knockdown

A shRNA construct targeting CKS2 (hairpin sequence: TGCTGTTGACAGTGAGCGAACAGCAACAGAGCTCAGTTAATAGTGAAGCCACAG ATGTATTAACTGAGCTCTGTTGCTGTGTGCCTACTGCCTCGGA (SEQ. ID. NO. 21)) in the pGIPZ backbone was obtained as a gift from the Paddison Lab (Fred Hutchinson Cancer Research Center). The shCKS2 construct was transfected into the CKS2 Mutant 2 clonal cell line created by CRISPR base editing due to its high endogenous expression of CKS2. Transfection was performed by plating 375,000 cells per well in 6-well plates, incubating overnight at 37° C., and next day adding 1.5 μg of plasmid DNA with 4.5 μL Fugene HD (Promega) according to manufacturer's instructions. 24 hours post-transfection of shCKS2, cells were harvested and lysed for Western blotting.

### Western Blotting

1×106 cells were collected from each CKS2 WT and Mutant 293T cell line and lysed in RIPA lysis buffer (Thermo Scientific) supplemented with 10% Complete Mini protease inhibitor (Sigma) and 10% PhosSTOP phosphatase inhibitor (Roche). After incubating on ice for 30 minutes, lysates were centrifuged at 13,000 g for 10 minutes at 4° C. The supernatant was collected and protein concentration measured using a Bradford assay (BioRad). 25-50 μg of extract per cell line was separated by SDS-PAGE and transferred onto PVDF membranes for immunoblot analysis. Primary antibodies used were CKS2 (Abcam 155078, 1:1000) and β-actin (Sigma 5316, 1:1000).

### Bioinformatics Analysis

### Obtaining Publicly Available 5′ UTR Sequencing Data Used for Analysis

BAM files 101 tumor/matched normal castration-resistant prostate cancer metastases patients were obtained from Quigely et al. and bedtools “bamtofastq” (https://bedtools.readthedocs.io/en/latest/content/tools/bamtofastq.html) was used to extract raw sequencing data from BAM files. FASTQ files for 262 tumor/matched normal patients were downloaded from Fraser et al.
(https://www.ebi.ac.uk/ega/datasets/EGAD00001003139). Whole genome sequencing (WGS) bam files for 20 tumor/matched normal localized prostate cancer patients were downloaded from the TCGA (https://portal.gdc.cancer.gov/legacy-archive).

### Genomic UTR Sequencing Alignment and Quality Control

Raw sequencing reads produced by Illumina's bcl2fastq 1.8.4 software were processed to exclude read pairs failing default (PF filtering) quality checks. FastQC
(https://www.bioinformatics.babraham.ac.uk/projects/fastqc/) was used to evaluate raw sequencing reads. All reads were aligned to hg19 using Bowtie, only reads aligning to standard chromosomes (chr 1:22, X,Y,M) were retained for further analysis The reads with low quality were filtered and duplicates were marked using Picard. 5′ UTR and CDS coverage was calculated using GATK “DepthOfCoverage” (https://gatk.broadinstitute.org/hc/en-us/articles/360041851491-DepthOfCoverage). ContEst was used to estimate the level of cross-individual contamination in matched normal-tumor pairs from all mCRPC patients and PDX samples that were sequenced.

### Mouse Subtraction for Human Patient Derived Xenograft 5′ UTR Mutation Calling

Short reads from the LuCaP PDX specimens were aligned to both human reference genome hg19 and mouse reference genome mm9 separately using TopHat⁵² (version 2.0.14). An in-house developed software was applied to retain the reads with higher fidelity to hg19 for further downstream analysis.

### Somatic Mutation Analysis

MuTect v1 and Strelka version 1.0.15 were used to identify somatic single nucleotide variants within the 5′ UTR and CDS for each tumor and matched normal pair. Two different bed files were used in two separate runs for obtaining 5′ UTR mutations and CDS mutations,
120501_hg19_RefSeq_Ensembl_UTRs_EZ_HX1_target_regions.bed and
120430_b37_ExomeV3_UTR_EZ_HX1_Union.bed respectively. For the University of Washington rapid autopsy specimens, LuCaP, TCGA and ICGC samples, the following cutoff was applied to derive a final list of mutations: total number of reads in tumor >14, and >8 in matched normal sample, number of reads aligning to alt allele in tumor >=4 and tumor variant allele frequency (VAF) >0.1. VAF refers to the fraction of sequencing reads overlapping a genomic coordinate that support the non-reference (mutant/alternate) allele.

### Ribosome Profiling Data Analysis

Libraries were sequenced on Illumina HiSeq 2500 at the Genomics Shared Resource in the FHCRC. The raw sequence data was uncompressed followed by clipping the 3′ adaptor sequence (AGATCGGAAGAGCACACGTCT (SEQ. ID. NO. 22)). Next, the trimmed sequence reads were aligned to human rRNA reference using Bowtie. The unaligned reads were collected while the rRNA alignments were discarded to reduce rRNA contamination. TopHatv2 was used to align the non-rRNA sequencing reads to hg19 and subtraction of mouse sequences were performed using a custom script. Aligned reads were counted for gene associations against the UCSC genes database with HTSeq. Five LuCaP and five normal prostate tissue samples were sequenced twice. In each analysis, two replicates for each LuCaP were considered as the test group and five normal prostate tissue samples as the control group. Xtail and DESeq2 were both used to find translationally regulated genes individually for each LuCaP (FDR<0.1 and fold change>1.5). Translation fold-changes were highly correlated across both packages. Similarly, DESeq2 was used to find transcriptionally regulated genes individually for each LuCaP (FDR<0.05 and fold change>2), which were excluded from the translationally regulated gene lists. R/Bioconductor package, riboseqR (http://bioconductor.org/packages/release/bioc/html/riboSeqR.html) was used to calculate triplet periodicity in all samples. Gene Set Enrichment Analysis (GSEA) was done using Broad's website for GSEA (http://www.gsea-msigdb.org/gsea/msigdb/index.jsp) using the MSigDb database.

### Extracting 5′ UTR Sequences

Using R/Bioconductor package GenomicFeatures transcript ids, genomic coordinates and transcription start sites for 5′ UTR of each of the mutated genes were obtained from UCSC's Refseq Table. 5′ UTR sequences were retrieved using R/Bioconductor packages
“BSgenome.Hsapiens. UCSC.hg19”. Coverage for 5′ UTR coordinates for each transcript was calculated using bedtools coverage
(https://bedtools.readthedocs.io/en/latest/content/tools/coverage.html). Median coverage for 200 bp flanking putative transcription start sites were plotted using R.

### Cis-regulatory Element Mutational Analysis

Analysis of 5′ UTR mutations within cis-element regulatory regions was performed by examining if the observed mutations in the patient cohort disrupt DNA binding element motifs, and other known elements, including RNA-binding protein binding sites, upstream start codons (uAUGs), terminal oligopyrimidine motifs (TOP)-like/Pyrimidine-Rich Translational Elements (PRTEs), G-quadruplexes, and 5′ Terminal Oligopyrimidine motifs (5′ TOP). A custom set of Python scripts
(available here: https://github.com/lukascorey/5-UTR-Mutation-Analysis) was written to determine whether the observed counts of 5′ UTR mutations were statistically enriched within these regulatory elements when compared to a random model preserving sequence-specific characteristics such as trinucleotide context. In this analysis, mutations that impacted pre-existing regulatory elements or introduced new elements were both considered. To generate the background distribution, permutations of all 5′ UTR mutation locations found within the dataset were performed ˜10,000 times. The original mutational frequency of all specific transversions, transitions and trinucleotide context (a total of 288 possible mutations are possible under this scheme—64 possible codons plus 32 additional with no nucleotide in exclusively the first or third position, each with three possible mutations to the middle base) were taken into account. The number of mutations in these permutations that affected each type of motif in each database or specific element were counted. The total number of observed mutations impacting each regulatory element type was compared to the background distribution of the permutation data and the p-value value was computed. We selected elements with p<0.025 as being statistically significant.

### DNA Binding Elements

Position weighted matrices of DNA binding elements were retrieved from the HOMER database. Position frequency of all known motifs in these databases were converted to Position Weighted Matrices using the standard conversion (log2(frequency/.25)). A total of 332 motifs were obtained from HOMER. All analysis with these motifs used a cutoff at 90 percent of the maximum score. Both the forward and reverse strands were scanned.

### RNA Binding Proteins

Position weighted matrices of RNA binding protein binding sites were retrieved from the Hughes lab dataset. Similarly, position frequency of all known human motifs in these databases were converted to Position Weighted Matrices using the standard conversion (log2(frequency/.25)). The analysis included 102 motifs from the Hughes database, with a 90 percent cutoff.

### Translational Regulatory Elements

To assess the functional impact on upstream open reading frames (uORFs), predicted functional uORFs were used. Observed 5′ UTR mutations from the dataset or the simulated permutations that landed within a start codon of one of these predicted reading frames was counted as mutating a uORF. The pyrimidine-rich translational element (PRTE) motif consists of an invariant uridine at position 6 flanked by pyrimidines and does not reside at position +1 of the 5′ UTR and is similar to the TOP-like sequence. The provided position weighted matrix was used to identify PRTEs. 5′ Terminal OligoPyrimidine Tracts (5′ TOP) were characterized as regions at the 5′ end of a 5′ UTR beginning with a cytosine and followed by no fewer than four pyrimidines. Mutations in the first ten base pairs of a UTR with a 5′ TOP were counted as mutating that 5′ TOP. G quadruplexes, defined as regions with four groups of at least two adjacent guanines separated by loops of at least one nucleotide but no more than seven nucleotides, were also considered in this analysis. For all RNA binding proteins and translational regulatory elements, the analysis was performed on the single-stranded mRNA plus strand.

### PLUMAGE Long-read Sequencing Analysis

Associations between inline 30-bp barcodes and specific 5′ UTR sequences were established by long-read sequencing on the PacBio Sequel system using four SMRT cells. An additional SMRT cell was dedicated to a smaller pool of 5′ UTR targets, containing Sal1 restriction sites, that were expected to be truncated in the main pool.

Subreads for each sequenced molecule were combined to form high-quality circular consensus sequences (CCS) using PacBio's Circular Consensus Sequencing 2 (CSS2) algorithm with default parameters (PacBio SMRTLink 6.0.0.47841, minimum 3 passes, minimum predicted accuracy 0.9). Within each CCS sequence, we identified the 5′ UTR and associated 30-bp barcode by searching for flanking 20-bp sequences expected to be constant across all constructs. CCS sequences where these flanking sequences were not found, or where a barcode had not been inserted and the EcoRI target sequence GAATTC remained, were excluded from further consideration.

Where available 5′ UTR sequences that share the same barcode were combined by multiple alignment with MUSCLE (v3.8.31), generating a single consensus sequence for each observed barcode. Consensus 5′ UTR sequences were annotated by exact matching to the PLUMAGE sequences submitted for synthesis. Exact matching is required because majority of the mutants differ by only a single base from the wild type. Consensus sequences that did not match exactly any PLUMAGE sequence were annotated to nearest PLUMAGE gene by blastn search, allowing us to identify genes whose 5′ UTRs may be difficult to sequence due to composition, repeats, or length.

The above process generated 968,990 CCS2 sequences containing 330,199 distinct 30-bp barcodes. Of these, 212,325 where associated with an exact match to an expected PLUMAGE 5′ UTR sequence. On average, annotated 5′ UTR sequences are supported by 236 distinct 30-bp barcodes (median is 200). Of the remaining 117,874 barcodes that did not match an expected 5′ UTR, 50% were supported by a single CCS2 sequence only so that multiple independent CCS2 sequences were unavailable for multiple alignment and further refinement. All unique 30-bp barcodes associated with each correctly synthesized 5′ UTR sequences were identified and used in the short-read sequencing analysis.

### PLUMAGE Short-read Sequence Analysis

To quantify 5′ UTR sequences in DNA, total mRNA, and polysome-bound mRNA, each sample was sequenced in triplicate on an Illumina HiSeq 2500 (PE100). Sequencing targeted only the barcode region of each sample ensuring that the barcode was completely contained within, and at a fixed offset from the 3′ end of the second 100 nt read in each pair. Barcodes were extracted from this fixed position, subject to the constraint that a short sequence (4 nt) on both sides match the expected sequence as a check on improper barcode length or placement. Using this method barcodes were extracted from 80% of the reads in each sample, and more than 96% of the extracted barcodes matched one previously cataloged by PacBio long-read sequencing. Between 6.4 and 16.5 million cataloged barcodes were assigned to each sample in this way. Extracted barcodes were tallied against corresponding 5′ UTRs using the barcode-to-variant mapping generated from PacBio long-read sequencing. To determine robustness of the assay, for each cell line, the number of times each barcode was observed, and the total number of barcodes observed for each 5′ UTR in each sample were counted. In addition to tables of raw counts, we produced counts-per-million (CPM) scaled summaries wherein raw counts were divided by the total number of reads (in millions) matched to barcodes in each sample to account for variation in sequencing depth. For each barcode, raw read counts were normalized by counts per million (CPM) within each sample for each biological replicate. All barcodes in each sample used in the calculation of ratios had a minimum normalized read count of 0.5 CPM. To determine changes in transcription, the log2 (Total mRNA/DNA) CPM was calculated for each barcode within each biological replicate, and all the barcodes for the mutant 5′ UTR were compared to the corresponding wild-type 5′ UTR. A two-sided Mann-Whitney U test was performed for each mutant and wild-type 5′ UTR using the R function Wilcox test and p-values were adjusted for multiple comparisons using the false discovery rate (FDR) method. Significance was determined by using a cutoff of FDR<0.1. To determine changes in mRNA translation efficiency or polysome to 80 S ratio, the log2 (polysome/Total mRNA or polysome/80 S) CPM was calculated for each barcode, and differences in mutant vs wild-type 5′ UTRs were determined in a similar manner. Significance was also determined by using a cutoff of FDR<0.1. To demonstrate reproducibility, scatter plots of normalized counts for each unique barcode were made comparing each sample for each biological replicate. The Pearson correlation was calculated for each comparison using R function cor( ). Density plots were made to represent normalized counts per barcode per sample using the R package ggplot2.

### Copy Number Analysis

Sequenza (v 2.1.9999b) was used to estimate allele-specific copy number calls, tumor cellularity and tumor ploidy for each tumor and its matched normal sample. Average depth ratio (tumor vs. normal) and B allele frequency (the lesser of the 2 allelic fractions as measured at germline heterozygous positions) was used to estimate copy number while considering the overall tumor ploidy/cellularity, genomic segment-specific copy number, and minor allele copy number. ˜150 bp sequences flanking the 5′ UTR mutation were considered.

### GSEA Analysis

MSigDB (v1.7) was used to compute overlaps with KEGG gene sets present in MSigDB database, gene sets with FDR<0.05 were considered significant. Fisher hypergeometric function were implemented in R using function phyper( ) to see if genes in one set were over-represented, compared to other gene sets.

### MAPK Network Visualization

MAPK signaling pathway (map0410) was downloaded from KEGG, Cytoscape (v 3.7.2, https://cytoscape.org/) was used to visualize the network where genes mutated in metastatic samples were colored in green and non-mutated genes were colored in grey.

### RNAseq Data Analysis

Raw sequencing from Nyquist et al. Cell Reports 2020, was aligned to hg19 using TopHat (v2), and aligned reads were counted for gene associations using HTSeq against the UCSC genes database. Normalized RNASeq data from Nyquist et al. 2020, mRNA samples from LuCaPs, were used to conduct a GSVA analysis for all C2 canonical pathways (KEGG, BIOCARTA, REACTOME) from MSigDb. A no-scale heatmap representing GSVA results for MAPK pathways was made using R package pheatmap (https://cran.r-project.org/web/packages/pheatmap/). With the same samples, GSVA analysis was also conducted using genes up-regulated in various mouse prostate tumors from Wang et al. Cancer Research 2012 and represented as a color-bar on the heatmap, as a MAPK pathway activity score.

### Statistical Analysis

All box plots and violin plots have the median as the center, and the first and third quartiles as the upper and lower edges of the box. All minimum and maximum data points are shown. Sample sizes, biological replicates and P values are indicated in relevant figures. All P values were obtained from two-tailed Student's t-tests, except for PLUMAGE validation experiments, where the one-tailed t-test was used to assess known directionality. A two-sided Mann-Whitney U test was performed for each mutant and wild-type 5′ UTR in PLUMAGE short-read sequencing data analysis. All p-values were adjusted for multiple comparisons using the false discovery rate (FDR) method. Pearson correlation was calculated for comparisons between replicates and cell lines. The fisher hypergeometric test was used to determine statistical significance between different gene sets.

While illustrative embodiments have been illustrated and described, it will be appreciated that various changes can be made therein without departing from the spirit and scope of the invention.

1. A method for analyzing an ability of target nucleic acid sequences to impact gene expression, the method comprising:
cloning the target nucleic acid sequences and associated barcode nucleic acid sequences into a plurality of plasmids;
sequencing the plurality of plasmids to provide long-read sequencing information based on a target nucleic acid sequence of the target nucleic acid sequences and an associated barcode nucleic acid sequence within a plasmid of the plurality of plasmids;
associating the target nucleic acid sequence with the associated barcode nucleic acid sequence based on the long-read sequencing information;
transducing the plurality of plasmids into a plurality of cells;
extracting DNA, total mRNA, and polysome-bound mRNA from the plurality of cells;
sequencing the barcode nucleic acid sequences in the extracted DNA, total mRNA, and polysome-bound mRNA to provide short-read sequencing information; and
analyzing the target nucleic acid sequences by comparing the long-read sequencing information and the short-read sequencing information.

2. The method of claim 1, wherein comparing the long-read sequencing information and the short-read sequencing information comprises:
associating barcodes detected in the short-read sequencing information from extracted DNA, total mRNA, and polysome-bound mRNA with the target nucleic acid sequences from the long-read sequencing information.

3. The method of claim 1, wherein analyzing the target nucleic acid sequences further comprises:
determining a number of target nucleic sequences, a number of RNA molecules translated from the target nucleic acid sequences, and a number of polysome-bound mRNA molecules from the long-read nucleic acid sequencing information and the short-read sequencing information.

4. The method of claim 3, wherein analyzing the target nucleic acid sequences further comprises:
quantitating mRNA transcript levels by determining a ratio of the number of RNA molecules translated from the target nucleic acid sequences to the number of target nucleic sequences.

5. The method of claim 4, wherein analyzing the target nucleic acid sequences further comprises:
comparing mRNA transcript levels of a wild-type target nucleic acid sequence to mRNA transcript levels of a mutant target nucleic acid sequence.

6. The method of claim 3, wherein analyzing the target nucleic acid sequences further comprises:
quantitating mRNA translation levels by determining a ratio of the number of polysome-bound mRNA molecules to the number of RNA molecules translated from the target nucleic acid sequences.

7. The method of claim 6, wherein analyzing the target nucleic acid sequences further comprises:
comparing mRNA translation levels of a mutant target nucleic acid sequence to mRNA translation levels of a wild-type target nucleic acid sequence.

8. The method of claim 1, wherein the target nucleic acid sequences include one or more untranslated regions (UTRs).

9. The method of claim 8, wherein the one or more UTRs are selected from a 5′ UTR, a 3′ UTR, and combinations thereof.

10. The method of claim 1, wherein a target nucleic acid sequence of the target nucleic acid sequences has a length in a range of about 40 base pairs to about 3,000 base pairs.

11. The method of claim 1, wherein the plasmid further comprises a promoter sequence.

12. The method of claim 11, wherein the promoter nucleic acid sequence is disposed at a 5′ end of the target nucleic acid sequence.

13. The method of claim 1, wherein the plasmid further comprises a reporter nucleic acid sequence.

14. The method of claim 13, wherein the reporter nucleic acid sequence is disposed at a 3′ end of the target nucleic acid sequence.

15. The method of claim 13, wherein the reporter nucleic acid sequence is disposed at a 5′ end of the barcode nucleic acid sequence.

16. The method of claim 1, wherein the barcode nucleic acid sequences include nucleic acid sequences selected from the group consisting of a random nucleic acid sequence, a concatenation of a plurality of barcode nucleic acid sequences, and combinations thereof.

17. The method of claim 1, wherein the method further comprises introducing a plurality of mutations into a plasmid of the plurality of plasmids.

18. The method of claim 1 wherein the method further comprising confirming the analyzed target nucleic acid sequences with a process selected from clustered regularly interspaced short palindromic repeats (CRISPR)-mediated base editing and prime editing.

19. A method for analyzing an ability of target nucleic acid sequences to impact gene expression, the method comprising:
cloning the target nucleic acid sequences and associated barcode nucleic acid sequences into a plurality of plasmids;
sequencing the plurality of plasmids to provide long-read sequencing information based on a target nucleic acid sequence of the target nucleic acid sequences and an associated barcode nucleic acid sequence within a plasmid of the plurality of plasmids;
associating the target nucleic acid sequence with the associated barcode nucleic acid sequence based on the long-read sequencing information;
transducing the plurality of plasmids into a plurality of cells;
extracting DNA, total mRNA, and polysome-bound mRNA from the plurality of cells;
sequencing the barcode nucleic acid sequences in the extracted DNA, total mRNA, and polysome-bound mRNA to provide short-read sequencing information;
analyzing the target nucleic acid sequences by comparing the long-read sequencing information and the short-read sequencing information;
determining a number of target nucleic sequences, a number of RNA molecules translated from the target nucleic acid sequences, and a number of polysome-bound mRNA molecules from the long-read nucleic acid sequencing information and the short-read sequencing information;
quantitating mRNA transcript levels by determining a ratio of the number of RNA molecules translated from the target nucleic acid sequences to the number of target nucleic sequences; and
comparing mRNA transcript levels of a wild-type target nucleic acid sequence to mRNA transcript levels of a mutant target nucleic acid sequence

20. A method for analyzing an ability of target nucleic acid sequences to impact gene expression, the method comprising:
cloning the target nucleic acid sequences and associated barcode nucleic acid sequences into a plurality of plasmids;
sequencing the plurality of plasmids to provide long-read sequencing information based on a target nucleic acid sequence of the target nucleic acid sequences and an associated barcode nucleic acid sequence within a plasmid of the plurality of plasmids;
associating the target nucleic acid sequence with the associated barcode nucleic acid sequence based on the long-read sequencing information;
transducing the plurality of plasmids into a plurality of cells;
extracting DNA, total mRNA, and polysome-bound mRNA from the plurality of cells;
sequencing the barcode nucleic acid sequences in the extracted DNA, total mRNA, and polysome-bound mRNA to provide short-read sequencing information;
analyzing the target nucleic acid sequences by comparing the long-read sequencing information and the short-read sequencing information;
determining a number of target nucleic sequences, a number of RNA molecules translated from the target nucleic acid sequences, and a number of polysome-bound mRNA molecules from the long-read nucleic acid sequencing information and the short-read sequencing information;
quantitating mRNA translation levels by determining a ratio of the number of polysome-bound mRNA molecules to the number of RNA molecules translated from the target nucleic acid sequences; and
comparing mRNA translation levels of a mutant target nucleic acid sequence to mRNA translation levels of a wild-type target nucleic acid sequence.


SEQUENCE LISTING (22 sequences)

SEQ ID NO: 1 - DNA, 57 bp, synthetic construct
  source 1..57: mol_type = other DNA
ccgatctgcgtgacataactaattacatgnnnnnnnntctagaattacacggcgatc

SEQ ID NO: 2 - DNA, 80 bp, synthetic construct
  source 1..80: mol_type = other DNA
ccgatctgcgtgacataactaattacatgawswswswswswswswswswswswswswswstctagaattacacggcgatc

SEQ ID NO: 3 - DNA, 33 bp, synthetic construct
  source 1..33: mol_type = other DNA
cacacaagcacacacgcgctcacacacagagag

SEQ ID NO: 4 - DNA, 33 bp, synthetic construct
  source 1..33: mol_type = other DNA
cacacaagcacacacgtgctcacacacagagag

SEQ ID NO: 5 - RNA, 23 nt, synthetic construct
  source 1..23: mol_type = other RNA
ctggscgtggttttgtctgctgc

SEQ ID NO: 6 - RNA, 21 nt, synthetic construct
  source 1..21: mol_type = other RNA
gtgttgggcgcgttatttatc

SEQ ID NO: 7 - RNA, 22 nt, synthetic construct
  source 1..22: mol_type = other RNA
taggctgcgaaatgttcatact

SEQ ID NO: 8 - RNA, 21 nt, synthetic construct
  source 1..21: mol_type = other RNA
cactacgagtaccggcatgtt

SEQ ID NO: 9 - RNA, 20 nt, synthetic construct
  source 1..20: mol_type = other RNA
accaagtctcctccactcct

SEQ ID NO: 10 - RNA, 20 nt, synthetic construct
  source 1..20: mol_type = other RNA
aaatctggcaccacaccttc

SEQ ID NO: 11 - RNA, 20 nt, synthetic construct
  source 1..20: mol_type = other RNA
ggggtgttgaaggtctcaaa

SEQ ID NO: 12 - DNA, 202 bp, synthetic construct
  source 1..202: mol_type = other DNA
aagtaccgaaaggtcttaccggaaaactcgacgcaagaaaaatcagagagatcctcataaaggccaagaagggcggaaagatcgccgtgtaattctagagaattctcatgtaattagttatgtcacgcagatcggaagagcgtcggggcggccggccgcttcgagcagacatgataagatacattgatgagtttggacaaac

SEQ ID NO: 13 - DNA, 36 bp, unidentified
  source 1..36: mol_type = other DNA
gaggaggcttttttggaggcctaggcttttgcaaaa

SEQ ID NO: 14 - DNA, 36 bp, synthetic construct
  source 1..36: mol_type = other DNA
catggaagacgccaaaaacataaagaaaggcccggc

SEQ ID NO: 15 - DNA, 56 bp, synthetic construct
  source 1..56: mol_type = other DNA
acactctttccctacacgacgctcttccgatctgcgtgacataactaattacatga

SEQ ID NO: 16 - RNA, 33 nt, synthetic construct
  source 1..33: mol_type = other RNA
ttttgtctgcgttttagagctagaaatagcaag

SEQ ID NO: 17 - RNA, 29 nt, synthetic construct
  source 1..29: mol_type = other RNA
ccacgtccagggtgtttcgtcctttccac

SEQ ID NO: 18 - RNA, 20 nt, synthetic construct
  source 1..20: mol_type = other RNA
ctggacgtggttttgtctgc

SEQ ID NO: 19 - RNA, 20 nt, synthetic construct
  source 1..20: mol_type = other RNA
acttccgcagaaggtgattg

SEQ ID NO: 20 - RNA, 22 nt, synthetic construct
  source 1..22: mol_type = other RNA
tactcgtagtgttcgtcgaagt

SEQ ID NO: 21 - RNA, 97 nt, synthetic construct
  source 1..97: mol_type = other RNA
tgctgttgacagtgagcgaacagcaacagagctcagttaatagtgaagccacagatgtattaactgagctctgttgctgtgtgcctactgcctcgga

SEQ ID NO: 22 - DNA, 21 bp, synthetic construct
  source 1..21: mol_type = other DNA
agatcggaagagcacacgtct